SEQUENCE LISTING (1291 sequences)

SEQ ID NO: 1 - DNA, 21 bp, Homo sapiens
aaccaaccaataatctcccac

SEQ ID NO: 2 - DNA, 21 bp, Homo sapiens
accaaccaattatctcccacc

SEQ ID NO: 3 - DNA, 21 bp, Homo sapiens
ccaaccaatattctcccaccc

SEQ ID NO: 4 - DNA, 21 bp, Homo sapiens
caaccaataatctcccacccc

SEQ ID NO: 5 - DNA, 21 bp, Homo sapiens
aaccaataatttcccacccca

SEQ ID NO: 6 - DNA, 21 bp, Homo sapiens
accaataatctcccaccccac

SEQ ID NO: 7 - DNA, 21 bp, Homo sapiens
ccaataatcttccaccccacc

SEQ ID NO: 8 - DNA, 21 bp, Homo sapiens
caataatctctcaccccacct

SEQ ID NO: 9 - DNA, 21 bp, Homo sapiens
aataatctcctaccccaccta

SEQ ID NO: 10 - DNA, 21 bp, Homo sapiens
ataatctccctccccacctaa

SEQ ID NO: 11 - DNA, 21 bp, Homo sapiens
taatctcccatcccacctaac

SEQ ID NO: 12 - DNA, 21 bp, Homo sapiens
aatctcccactccacctaact

SEQ ID NO: 13 - DNA, 21 bp, Homo sapiens
atctcccacctcacctaactc

SEQ ID NO: 14 - DNA, 21 bp, Homo sapiens
tctcccaccctacctaactca

SEQ ID NO: 15 - DNA, 21 bp, Homo sapiens
ctcccacccctcctaactcac

SEQ ID NO: 16 - DNA, 21 bp, Homo sapiens
tcccaccccatctaactcaca

SEQ ID NO: 17 - DNA, 21 bp, Homo sapiens
cccaccccacttaactcacac

SEQ ID NO: 18 - DNA, 21 bp, Homo sapiens
ccaccccacctaactcacaca

SEQ ID NO: 19 - DNA, 21 bp, Homo sapiens
caccccaccttactcacacaa

SEQ ID NO: 20 - DNA, 21 bp, Homo sapiens
accccacctatctcacacaaa

SEQ ID NO: 21 - DNA, 21 bp, Homo sapiens
ccccacctaattcacacaaac

SEQ ID NO: 22 - DNA, 21 bp, Homo sapiens
cccacctaactcacacaaacc

SEQ ID NO: 23 - DNA, 21 bp, Homo sapiens
ccacctaacttacacaaacca

SEQ ID NO: 24 - DNA, 21 bp, Homo sapiens
cacctaactctcacaaaccac

SEQ ID NO: 25 - DNA, 21 bp, Homo sapiens
acctaactcatacaaaccacc

SEQ ID NO: 26 - DNA, 20 bp, Homo sapiens
atatagtttcgtcattcatc

SEQ ID NO: 27 - DNA, 20 bp, Homo sapiens
tacattgcccatgtaattaa

SEQ ID NO: 28 - DNA, 20 bp, Homo sapiens
atatagtttcgtcattcatc

SEQ ID NO: 29 - DNA, 20 bp, Homo sapiens
tacattgcccatgtaattaa

SEQ ID NO: 30 - DNA, 20 bp, Homo sapiens
agatagttttgtcattcatc

SEQ ID NO: 31 - DNA, 20 bp, Homo sapiens
agatagtttcttcattcatc

SEQ ID NO: 32 - DNA, 20 bp, Homo sapiens
agatagtttcgtcattcatc

SEQ ID NO: 33 - DNA, 20 bp, Homo sapiens
agatagtttcgttattcatc

SEQ ID NO: 34 - DNA, 21 bp, Homo sapiens
cctaactcactcaaaccacca

SEQ ID NO: 35 - DNA, 21 bp, Homo sapiens
ctaactcacataaaccaccaa

SEQ ID NO: 36 - DNA, 21 bp, Homo sapiens
taactcacactaaccaccaac

SEQ ID NO: 37 - DNA, 21 bp, Homo sapiens
aaccaaccaagaatctcccac

SEQ ID NO: 38 - DNA, 21 bp, Homo sapiens
accaaccaatgatctcccacc

SEQ ID NO: 39 - DNA, 21 bp, Homo sapiens
ccaaccaatagtctcccaccc

SEQ ID NO: 40 - DNA, 21 bp, Homo sapiens
caaccaataagctcccacccc

SEQ ID NO: 41 - DNA, 21 bp, Homo sapiens
aaccaataatgtcccacccca

SEQ ID NO: 42 - DNA, 21 bp, Homo sapiens
accaataatcgcccaccccac

SEQ ID NO: 43 - DNA, 21 bp, Homo sapiens
ccaataatctgccaccccacc

SEQ ID NO: 44 - DNA, 21 bp, Homo sapiens
caataatctcgcaccccacct

SEQ ID NO: 45 - DNA, 21 bp, Homo sapiens
aataatctccgaccccaccta

SEQ ID NO: 46 - DNA, 21 bp, Homo sapiens
ataatctcccgccccacctaa

SEQ ID NO: 47 - DNA, 21 bp, Homo sapiens
taatctcccagcccacctaac

SEQ ID NO: 48 - DNA, 21 bp, Homo sapiens
aatctcccacgccacctaact

SEQ ID NO: 49 - DNA, 21 bp, Homo sapiens
atctcccaccgcacctaactc

SEQ ID NO: 50 - DNA, 21 bp, Homo sapiens
tctcccacccgacctaactca

SEQ ID NO: 51 - DNA, 21 bp, Homo sapiens
ctcccaccccgcctaactcac

SEQ ID NO: 52 - DNA, 21 bp, Homo sapiens
tcccaccccagctaactcaca

SEQ ID NO: 53 - DNA, 21 bp, Homo sapiens
cccaccccacgtaactcacac

SEQ ID NO: 54 - DNA, 21 bp, Homo sapiens
ccaccccaccgaactcacaca

SEQ ID NO: 55 - DNA, 21 bp, Homo sapiens
caccccacctgactcacacaa

SEQ ID NO: 56 - DNA, 21 bp, Homo sapiens
accccacctagctcacacaaa

SEQ ID NO: 57 - DNA, 21 bp, Homo sapiens
ccccacctaagtcacacaaac

SEQ ID NO: 58 - DNA, 21 bp, Homo sapiens
cccacctaacgcacacaaacc

SEQ ID NO: 59 - DNA, 21 bp, Homo sapiens
ccacctaactgacacaaacca

SEQ ID NO: 60 - DNA, 21 bp, Homo sapiens
cacctaactcgcacaaaccac

SEQ ID NO: 61 - DNA, 21 bp, Homo sapiens
acctaactcagacaaaccacc

SEQ ID NO: 62 - DNA, 20 bp, Homo sapiens
tacattgcccatgtaattaa

SEQ ID NO: 63 - DNA, 20 bp, Homo sapiens
atatagtttcgtcattcatc

SEQ ID NO: 64 - DNA, 20 bp, Homo sapiens
tacattgcccatgtaattaa

SEQ ID NO: 65 - DNA, 20 bp, Homo sapiens
atatagtttcgtcattcatc

SEQ ID NO: 66 - DNA, 20 bp, Homo sapiens
agatagtttggtcattcatc

SEQ ID NO: 67 - DNA, 20 bp, Homo sapiens
agatagtttcgtcattcatc

SEQ ID NO: 68 - DNA, 20 bp, Homo sapiens
agatagtttcggcattcatc

SEQ ID NO: 69 - DNA, 20 bp, Homo sapiens
agatagtttcgtgattcatc

SEQ ID NO: 70 - DNA, 21 bp, Homo sapiens
cctaactcacgcaaaccacca

SEQ ID NO: 71 - DNA, 21 bp, Homo sapiens
ctaactcacagaaaccaccaa

SEQ ID NO: 72 - DNA, 21 bp, Homo sapiens
taactcacacgaaccaccaac

SEQ ID NO: 73 - DNA, 21 bp, Homo sapiens
aaccaaccaacaatctcccac

SEQ ID NO: 74 - DNA, 21 bp, Homo sapiens
accaaccaatcatctcccacc

SEQ ID NO: 75 - DNA, 21 bp, Homo sapiens
ccaaccaatactctcccaccc

SEQ ID NO: 76 - DNA, 21 bp, Homo sapiens
caaccaataacctcccacccc

SEQ ID NO: 77 - DNA, 21 bp, Homo sapiens
aaccaataatctcccacccca

SEQ ID NO: 78 - DNA, 21 bp, Homo sapiens
accaataatcccccaccccac

SEQ ID NO: 79 - DNA, 21 bp, Homo sapiens
ccaataatctcccaccccacc

SEQ ID NO: 80 - DNA, 21 bp, Homo sapiens
caataatctcccaccccacct

SEQ ID NO: 81 - DNA, 21 bp, Homo sapiens
aataatctcccaccccaccta

SEQ ID NO: 82 - DNA, 21 bp, Homo sapiens
ataatctccccccccacctaa

SEQ ID NO: 83 - DNA, 21 bp, Homo sapiens
taatctcccaccccacctaac

SEQ ID NO: 84 - DNA, 21 bp, Homo sapiens
aatctcccaccccacctaact

SEQ ID NO: 85 - DNA, 21 bp, Homo sapiens
atctcccaccccacctaactc

SEQ ID NO: 86 - DNA, 21 bp, Homo sapiens
tctcccaccccacctaactca

SEQ ID NO: 87 - DNA, 21 bp, Homo sapiens
ctcccaccccccctaactcac

SEQ ID NO: 88 - DNA, 21 bp, Homo sapiens
tcccaccccacctaactcaca

SEQ ID NO: 89 - DNA, 21 bp, Homo sapiens
cccaccccacctaactcacac

SEQ ID NO: 90 - DNA, 21 bp, Homo sapiens
ccaccccacccaactcacaca

SEQ ID NO: 91 - DNA, 21 bp, Homo sapiens
caccccacctcactcacacaa

SEQ ID NO: 92 - DNA, 21 bp, Homo sapiens
accccacctacctcacacaaa

SEQ ID NO: 93 - DNA, 21 bp, Homo sapiens
ccccacctaactcacacaaac

SEQ ID NO: 94 - DNA, 21 bp, Homo sapiens
cccacctaacccacacaaacc

SEQ ID NO: 95 - DNA, 21 bp, Homo sapiens
ccacctaactcacacaaacca

SEQ ID NO: 96 - DNA, 21 bp, Homo sapiens
cacctaactcccacaaaccac

SEQ ID NO: 97 - DNA, 21 bp, Homo sapiens
acctaactcacacaaaccacc

SEQ ID NO: 98 - DNA, 20 bp, Homo sapiens
atatagtttcgtcattcatc

SEQ ID NO: 99 - DNA, 20 bp, Homo sapiens
tacattgcccatgtaattaa

SEQ ID NO: 100 - DNA, 20 bp, Homo sapiens
atatagtttcgtcattcatc

SEQ ID NO: 101 - DNA, 20 bp, Homo sapiens
tacattgcccatgtaattaa

SEQ ID NO: 102 - DNA, 20 bp, Homo sapiens
agatagtttcgtcattcatc

SEQ ID NO: 103 - DNA, 20 bp, Homo sapiens
agatagtttcctcattcatc

SEQ ID NO: 104 - DNA, 20 bp, Homo sapiens
agatagtttcgccattcatc

SEQ ID NO: 105 - DNA, 20 bp, Homo sapiens
agatagtttcgtcattcatc

SEQ ID NO: 106 - DNA, 21 bp, Homo sapiens
cctaactcacccaaaccacca

SEQ ID NO: 107 - DNA, 21 bp, Homo sapiens
ctaactcacacaaaccaccaa

SEQ ID NO: 108 - DNA, 21 bp, Homo sapiens
taactcacaccaaccaccaac

SEQ ID NO: 109 - DNA, 21 bp, Homo sapiens
aaccaaccaaaaatctcccac

SEQ ID NO: 110 - DNA, 21 bp, Homo sapiens
accaaccaataatctcccacc

SEQ ID NO: 111 - DNA, 21 bp, Homo sapiens
ccaaccaataatctcccaccc

SEQ ID NO: 112 - DNA, 21 bp, Homo sapiens
caaccaataaactcccacccc

SEQ ID NO: 113 - DNA, 21 bp, Homo sapiens
aaccaataatatcccacccca

SEQ ID NO: 114 - DNA, 21 bp, Homo sapiens
accaataatcacccaccccac

SEQ ID NO: 115 - DNA, 21 bp, Homo sapiens
ccaataatctaccaccccacc

SEQ ID NO: 116 - DNA, 21 bp, Homo sapiens
caataatctcacaccccacct

SEQ ID NO: 117 - DNA, 21 bp, Homo sapiens
aataatctccaaccccaccta

SEQ ID NO: 118 - DNA, 21 bp, Homo sapiens
ataatctcccaccccacctaa

SEQ ID NO: 119 - DNA, 21 bp, Homo sapiens
taatctcccaacccacctaac

SEQ ID NO: 120 - DNA, 21 bp, Homo sapiens
aatctcccacaccacctaact

SEQ ID NO: 121 - DNA, 21 bp, Homo sapiens
atctcccaccacacctaactc

SEQ ID NO: 122 - DNA, 21 bp, Homo sapiens
tctcccacccaacctaactca

SEQ ID NO: 123 - DNA, 21 bp, Homo sapiens
ctcccaccccacctaactcac

SEQ ID NO: 124 - DNA, 21 bp, Homo sapiens
tcccaccccaactaactcaca

SEQ ID NO: 125 - DNA, 21 bp, Homo sapiens
cccaccccacataactcacac

SEQ ID NO: 126 - DNA, 21 bp, Homo sapiens
ccaccccaccaaactcacaca

SEQ ID NO: 127 - DNA, 21 bp, Homo sapiens
caccccacctaactcacacaa

SEQ ID NO: 128 - DNA, 21 bp, Homo sapiens
accccacctaactcacacaaa

SEQ ID NO: 129 - DNA, 21 bp, Homo sapiens
ccccacctaaatcacacaaac

SEQ ID NO: 130 - DNA, 21 bp, Homo sapiens
cccacctaacacacacaaacc

SEQ ID NO: 131 - DNA, 21 bp, Homo sapiens
ccacctaactaacacaaacca

SEQ ID NO: 132 - DNA, 21 bp, Homo sapiens
cacctaactcacacaaaccac

SEQ ID NO: 133 - DNA, 21 bp, Homo sapiens
acctaactcaaacaaaccacc

SEQ ID NO: 134 - DNA, 20 bp, Homo sapiens
tacattgcccatgtaattaa

SEQ ID NO: 135 - DNA, 20 bp, Homo sapiens
atatagtttcgtcattcatc

SEQ ID NO: 136 - DNA, 20 bp, Homo sapiens
tacattgcccatgtaattaa

SEQ ID NO: 137 - DNA, 20 bp, Homo sapiens
atatagtttcgtcattcatc

SEQ ID NO: 138 - DNA, 20 bp, Homo sapiens
agatagtttagtcattcatc

SEQ ID NO: 139 - DNA, 20 bp, Homo sapiens
agatagtttcatcattcatc

SEQ ID NO: 140 - DNA, 20 bp, Homo sapiens
agatagtttcgacattcatc

SEQ ID NO: 141 - DNA, 20 bp, Homo sapiens
agatagtttcgtaattcatc

SEQ ID NO: 142 - DNA, 21 bp, Homo sapiens
cctaactcacacaaaccacca

SEQ ID NO: 143 - DNA, 21 bp, Homo sapiens
ctaactcacaaaaaccaccaa

SEQ ID NO: 144 - DNA, 21 bp, Homo sapiens
taactcacacaaaccaccaac

SEQ ID NO: 145 - DNA, 21 bp, Homo sapiens
aactcacacataccaccaaca

SEQ ID NO: 146 - DNA, 21 bp, Homo sapiens
actcacacaatccaccaacac

SEQ ID NO: 147 - DNA, 21 bp, Homo sapiens
ctcacacaaatcaccaacacc

SEQ ID NO: 148 - DNA, 21 bp, Homo sapiens
tcacacaaactaccaacacct

SEQ ID NO: 149 - DNA, 21 bp, Homo sapiens
cacacaaacctccaacacctc

SEQ ID NO: 150 - DNA, 21 bp, Homo sapiens
acacaaaccatcaacacctct

SEQ ID NO: 151 - DNA, 21 bp, Homo sapiens
cacaaaccactaacacctctc

SEQ ID NO: 152 - DNA, 21 bp, Homo sapiens
acaaaccacctacacctctcc

SEQ ID NO: 153 - DNA, 21 bp, Homo sapiens
caaaccaccatcacctctccc

SEQ ID NO: 154 - DNA, 21 bp, Homo sapiens
aaaccaccaatacctctcccc

SEQ ID NO: 155 - DNA, 21 bp, Homo sapiens
aaccaccaactcctctccccc

SEQ ID NO: 156 - DNA, 21 bp, Homo sapiens
accaccaacatctctccccct

SEQ ID NO: 157 - DNA, 21 bp, Homo sapiens
ccaccaacacttctccccctc

SEQ ID NO: 158 - DNA, 21 bp, Homo sapiens
caccaacacctctccccctct

SEQ ID NO: 159 - DNA, 21 bp, Homo sapiens
accaacacctttccccctctc

SEQ ID NO: 160 - DNA, 21 bp, Homo sapiens
ccaacacctctccccctctca

SEQ ID NO: 161 - DNA, 21 bp, Homo sapiens
caacacctcttcccctctcat

SEQ ID NO: 162 - DNA, 21 bp, Homo sapiens
aacacctctctccctctcatc

SEQ ID NO: 163 - DNA, 21 bp, Homo sapiens
acacctctcctcctctcatcc

SEQ ID NO: 164 - DNA, 21 bp, Homo sapiens
cacctctccctctctcatcca

SEQ ID NO: 165 - DNA, 21 bp, Homo sapiens
acctctccccttctcatccat

SEQ ID NO: 166 - DNA, 21 bp, Homo sapiens
cctctccccctctcatccatc

SEQ ID NO: 167 - DNA, 20 bp, Homo sapiens
atatagtttcgtcattcatc

SEQ ID NO: 168 - DNA, 20 bp, Homo sapiens
tacattgcccatgtaattaa

SEQ ID NO: 169 - DNA, 20 bp, Homo sapiens
atatagtttcgtcattcatc

SEQ ID NO: 170 - DNA, 20 bp, Homo sapiens
tacattgcccatgtaattaa

SEQ ID NO: 171 - DNA, 20 bp, Homo sapiens
agatagttttgtcattcatc

SEQ ID NO: 172 - DNA, 20 bp, Homo sapiens
agatagtttcttcattcatc

SEQ ID NO: 173 - DNA, 20 bp, Homo sapiens
agatagtttcgtcattcatc

SEQ ID NO: 174 - DNA, 20 bp, Homo sapiens
agatagtttcgttattcatc

SEQ ID NO: 175 - DNA, 21 bp, Homo sapiens
ctctccccctttcatccatca

SEQ ID NO: 176 - DNA, 21 bp, Homo sapiens
tctccccctctcatccatcac

SEQ ID NO: 177 - DNA, 21 bp, Homo sapiens
ctccccctcttatccatcacc

SEQ ID NO: 178 - DNA, 21 bp, Homo sapiens
tccccctctcttccatcaccc

SEQ ID NO: 179 - DNA, 21 bp, Homo sapiens
ccccctctcatccatcaccca

SEQ ID NO: 180 - DNA, 21 bp, Homo sapiens
cccctctcattcatcacccac

SEQ ID NO: 181 - DNA, 21 bp, Homo sapiens
aactcacacagaccaccaaca

SEQ ID NO: 182 - DNA, 21 bp, Homo sapiens
actcacacaagccaccaacac

SEQ ID NO: 183 - DNA, 21 bp, Homo sapiens
ctcacacaaagcaccaacacc

SEQ ID NO: 184 - DNA, 21 bp, Homo sapiens
tcacacaaacgaccaacacct

SEQ ID NO: 185 - DNA, 21 bp, Homo sapiens
cacacaaaccgccaacacctc

SEQ ID NO: 186 - DNA, 21 bp, Homo sapiens
acacaaaccagcaacacctct

SEQ ID NO: 187 - DNA, 21 bp, Homo sapiens
cacaaaccacgaacacctctc

SEQ ID NO: 188 - DNA, 21 bp, Homo sapiens
acaaaccaccgacacctctcc

SEQ ID NO: 189 - DNA, 21 bp, Homo sapiens
caaaccaccagcacctctccc

SEQ ID NO: 190 - DNA, 21 bp, Homo sapiens
aaaccaccaagacctctcccc

SEQ ID NO: 191 - DNA, 21 bp, Homo sapiens
aaccaccaacgcctctccccc

SEQ ID NO: 192 - DNA, 21 bp, Homo sapiens
accaccaacagctctccccct

SEQ ID NO: 193 - DNA, 21 bp, Homo sapiens
ccaccaacacgtctccccctc

SEQ ID NO: 194 - DNA, 21 bp, Homo sapiens
caccaacaccgctccccctct

SEQ ID NO: 195 - DNA, 21 bp, Homo sapiens
accaacacctgtccccctctc

SEQ ID NO: 196 - DNA, 21 bp, Homo sapiens
ccaacacctcgccccctctca

SEQ ID NO: 197 - DNA, 21 bp, Homo sapiens
caacacctctgcccctctcat

SEQ ID NO: 198 - DNA, 21 bp, Homo sapiens
aacacctctcgccctctcatc

SEQ ID NO: 199 - DNA, 21 bp, Homo sapiens
acacctctccgcctctcatcc

SEQ ID NO: 200 - DNA, 21 bp, Homo sapiens
cacctctcccgctctcatcca

SEQ ID NO: 201 - DNA, 21 bp, Homo sapiens
acctctccccgtctcatccat

SEQ ID NO: 202 - DNA, 21 bp, Homo sapiens
cctctcccccgctcatccatc

SEQ ID NO: 203 - DNA, 20 bp, Homo sapiens
tacattgcccatgtaattaa

SEQ ID NO: 204 - DNA, 20 bp, Homo sapiens
atatagtttcgtcattcatc

SEQ ID NO: 205 - DNA, 20 bp, Homo sapiens
tacattgcccatgtaattaa

SEQ ID NO: 206 - DNA, 20 bp, Homo sapiens
atatagtttcgtcattcatc

SEQ ID NO: 207 - DNA, 20 bp, Homo sapiens
agatagtttggtcattcatc

SEQ ID NO: 208 - DNA, 20 bp, Homo sapiens
agatagtttcgtcattcatc

SEQ ID NO: 209 - DNA, 20 bp, Homo sapiens
agatagtttcggcattcatc

SEQ ID NO: 210 - DNA, 20 bp, Homo sapiens
agatagtttcgtgattcatc

SEQ ID NO: 211 - DNA, 21 bp, Homo sapiens
ctctccccctgtcatccatca

SEQ ID NO: 212 - DNA, 21 bp, Homo sapiens
tctccccctcgcatccatcac

SEQ ID NO: 213 - DNA, 21 bp, Homo sapiens
ctccccctctgatccatcacc

SEQ ID NO: 214 - DNA, 21 bp, Homo sapiens
tccccctctcgtccatcaccc

SEQ ID NO: 215 - DNA, 21 bp, Homo sapiens
ccccctctcagccatcaccca

SEQ ID NO: 216 - DNA, 21 bp, Homo sapiens
cccctctcatgcatcacccac

SEQ ID NO: 217 - DNA, 21 bp, Homo sapiens
aactcacacacaccaccaaca

SEQ ID NO: 218 - DNA, 21 bp, Homo sapiens
actcacacaacccaccaacac

SEQ ID NO: 219 - DNA, 21 bp, Homo sapiens
ctcacacaaaccaccaacacc

SEQ ID NO: 220 - DNA, 21 bp, Homo sapiens
tcacacaaaccaccaacacct

SEQ ID NO: 221 - DNA, 21 bp, Homo sapiens
cacacaaacccccaacacctc

SEQ ID NO: 222 - DNA, 21 bp, Homo sapiens
acacaaaccaccaacacctct

SEQ ID NO: 223 - DNA, 21 bp, Homo sapiens
cacaaaccaccaacacctctc

SEQ ID NO: 224 - DNA, 21 bp, Homo sapiens
acaaaccacccacacctctcc

SEQ ID NO: 225 - DNA, 21 bp, Homo sapiens
caaaccaccaccacctctccc

SEQ ID NO: 226 - DNA, 21 bp, Homo sapiens
aaaccaccaacacctctcccc

SEQ ID NO: 227 - DNA, 21 bp, Homo sapiens
aaccaccaacccctctccccc

SEQ ID NO: 228 - DNA, 21 bp, Homo sapiens
accaccaacacctctccccct

SEQ ID NO: 229 - DNA, 21 bp, Homo sapiens
ccaccaacacctctccccctc

SEQ ID NO: 230 - DNA, 21 bp, Homo sapiens
caccaacacccctccccctct

SEQ ID NO: 231 - DNA, 21 bp, Homo sapiens
accaacacctctccccctctc

SEQ ID NO: 232 - DNA, 21 bp, Homo sapiens
ccaacacctccccccctctca

SEQ ID NO: 233 - DNA, 21 bp, Homo sapiens
caacacctctccccctctcat

SEQ ID NO: 234 - DNA, 21 bp, Homo sapiens
aacacctctccccctctcatc

SEQ ID NO: 235 - DNA, 21 bp, Homo sapiens
acacctctccccctctcatcc

SEQ ID NO: 236 - DNA, 21 bp, Homo sapiens
cacctctccccctctcatcca

SEQ ID NO: 237 - DNA, 21 bp, Homo sapiens
acctctccccctctcatccat

SEQ ID NO: 238 - DNA, 21 bp, Homo sapiens
cctctccccccctcatccatc

SEQ ID NO: 239 - DNA, 20 bp, Homo sapiens
atatagtttcgtcattcatc

SEQ ID NO: 240 - DNA, 20 bp, Homo sapiens
tacattgcccatgtaattaa

SEQ ID NO: 241 - DNA, 20 bp, Homo sapiens
atatagtttcgtcattcatc

SEQ ID NO: 242 - DNA, 20 bp, Homo sapiens
tacattgcccatgtaattaa

SEQ ID NO: 243 - DNA, 20 bp, Homo sapiens
agatagtttcgtcattcatc

SEQ ID NO: 244 - DNA, 20 bp, Homo sapiens
agatagtttcctcattcatc

SEQ ID NO: 245 - DNA, 20 bp, Homo sapiens
agatagtttcgccattcatc

SEQ ID NO: 246 - DNA, 20 bp, Homo sapiens
agatagtttcgtcattcatc

SEQ ID NO: 247 - DNA, 21 bp, Homo sapiens
ctctccccctctcatccatca

SEQ ID NO: 248 - DNA, 21 bp, Homo sapiens
tctccccctcccatccatcac

SEQ ID NO: 249 - DNA, 21 bp, Homo sapiens
ctccccctctcatccatcacc

SEQ ID NO: 250 - DNA, 21 bp, Homo sapiens
tccccctctcctccatcaccc

SEQ ID NO: 251 - DNA, 21 bp, Homo sapiens
ccccctctcacccatcaccca

SEQ ID NO: 252 - DNA, 21 bp, Homo sapiens
cccctctcatccatcacccac

SEQ ID NO: 253 - DNA, 21 bp, Homo sapiens
aactcacacaaaccaccaaca

SEQ ID NO: 254 - DNA, 21 bp, Homo sapiens
actcacacaaaccaccaacac

SEQ ID NO: 255 - DNA, 21 bp, Homo sapiens
ctcacacaaaacaccaacacc

SEQ ID NO: 256 - DNA, 21 bp, Homo sapiens
tcacacaaacaaccaacacct

SEQ ID NO: 257 - DNA, 21 bp, Homo sapiens
cacacaaaccaccaacacctc

SEQ ID NO: 258 - DNA, 21 bp, Homo sapiens
acacaaaccaacaacacctct

SEQ ID NO: 259 - DNA, 21 bp, Homo sapiens
cacaaaccacaaacacctctc

SEQ ID NO: 260 - DNA, 21 bp, Homo sapiens
acaaaccaccaacacctctcc

SEQ ID NO: 261 - DNA, 21 bp, Homo sapiens
caaaccaccaacacctctccc

SEQ ID NO: 262 - DNA, 21 bp, Homo sapiens
aaaccaccaaaacctctcccc

SEQ ID NO: 263 - DNA, 21 bp, Homo sapiens
aaccaccaacacctctccccc

SEQ ID NO: 264 - DNA, 21 bp, Homo sapiens
accaccaacaactctccccct

SEQ ID NO: 265 - DNA, 21 bp, Homo sapiens
ccaccaacacatctccccctc

SEQ ID NO: 266 - DNA, 21 bp, Homo sapiens
caccaacaccactccccctct

SEQ ID NO: 267 - DNA, 21 bp, Homo sapiens
accaacacctatccccctctc

SEQ ID NO: 268 - DNA, 21 bp, Homo sapiens
ccaacacctcaccccctctca

SEQ ID NO: 269 - DNA, 21 bp, Homo sapiens
caacacctctacccctctcat

SEQ ID NO: 270 - DNA, 21 bp, Homo sapiens
aacacctctcaccctctcatc

SEQ ID NO: 271 - DNA, 21 bp, Homo sapiens
acacctctccacctctcatcc

SEQ ID NO: 272 - DNA, 21 bp, Homo sapiens
cacctctcccactctcatcca

SEQ ID NO: 273 - DNA, 21 bp, Homo sapiens
acctctccccatctcatccat

SEQ ID NO: 274 - DNA, 21 bp, Homo sapiens
cctctcccccactcatccatc

SEQ ID NO: 275 - DNA, 20 bp, Homo sapiens
tacattgcccatgtaattaa

SEQ ID NO: 276 - DNA, 20 bp, Homo sapiens
atatagtttcgtcattcatc

SEQ ID NO: 277 - DNA, 20 bp, Homo sapiens
tacattgcccatgtaattaa

SEQ ID NO: 278 - DNA, 20 bp, Homo sapiens
atatagtttcgtcattcatc

SEQ ID NO: 279 - DNA, 20 bp, Homo sapiens
agatagtttagtcattcatc

SEQ ID NO: 280 - DNA, 20 bp, Homo sapiens
agatagtttcatcattcatc

SEQ ID NO: 281 - DNA, 20 bp, Homo sapiens
agatagtttcgacattcatc

SEQ ID NO: 282 - DNA, 20 bp, Homo sapiens
agatagtttcgtaattcatc

SEQ ID NO: 283 - DNA, 21 bp, Homo sapiens
ctctccccctatcatccatca

SEQ ID NO: 284 - DNA, 21 bp, Homo sapiens
tctccccctcacatccatcac

SEQ ID NO: 285 - DNA, 21 bp, Homo sapiens
ctccccctctaatccatcacc

SEQ ID NO: 286 - DNA, 21 bp, Homo sapiens
tccccctctcatccatcaccc

SEQ ID NO: 287 - DNA, 21 bp, Homo sapiens
ccccctctcaaccatcaccca

SEQ ID NO: 288 - DNA, 21 bp, Homo sapiens
cccctctcatacatcacccac

SEQ ID NO: 289 - DNA, 21 bp, Homo sapiens
ccctctcatctatcacccacc

SEQ ID NO: 290 - DNA, 21 bp, Homo sapiens
cctctcatccttcacccacca

SEQ ID NO: 291 - DNA, 21 bp, Homo sapiens
ctctcatccatcacccaccac

SEQ ID NO: 292 - DNA, 21 bp, Homo sapiens
tctcatccattacccaccacc

SEQ ID NO: 293 - DNA, 21 bp, Homo sapiens
ctcatccatctcccaccaccc

SEQ ID NO: 294 - DNA, 21 bp, Homo sapiens
tcatccatcatccaccacccc

SEQ ID NO: 295 - DNA, 21 bp, Homo sapiens
catccatcactcaccacccct

SEQ ID NO: 296 - DNA, 21 bp, Homo sapiens
atccatcacctaccacccctc

SEQ ID NO: 297 - DNA, 21 bp, Homo sapiens
tccatcaccctccacccctca

SEQ ID NO: 298 - DNA, 21 bp, Homo sapiens
ccatcacccatcacccctcat

SEQ ID NO: 299 - DNA, 21 bp, Homo sapiens
catcacccactacccctcatc

SEQ ID NO: 300 - DNA, 21 bp, Homo sapiens
atcacccacctcccctcatca

SEQ ID NO: 301 - DNA, 21 bp, Homo sapiens
tcacccaccatccctcatcat

SEQ ID NO: 302 - DNA, 21 bp, Homo sapiens
cacccaccactcctcatcata

SEQ ID NO: 303 - DNA, 21 bp, Homo sapiens
acccaccacctctcatcatac

SEQ ID NO: 304 - DNA, 21 bp, Homo sapiens
cccaccacccttcatcatacc

SEQ ID NO: 305 - DNA, 21 bp, Homo sapiens
ccaccacccctcatcatacct

SEQ ID NO: 306 - DNA, 21 bp, Homo sapiens
caccaccccttatcatacctc

SEQ ID NO: 307 - DNA, 21 bp, Homo sapiens
accacccctcttcatacctca

SEQ ID NO: 308 - DNA, 20 bp, Homo sapiens
atatagtttcgtcattcatc

SEQ ID NO: 309 - DNA, 20 bp, Homo sapiens
tacattgcccatgtaattaa

SEQ ID NO: 310 - DNA, 20 bp, Homo sapiens
atatagtttcgtcattcatc

SEQ ID NO: 311 - DNA, 20 bp, Homo sapiens
tacattgcccatgtaattaa

SEQ ID NO: 312 - DNA, 20 bp, Homo sapiens
agatagttttgtcattcatc

SEQ ID NO: 313 - DNA, 20 bp, Homo sapiens
agatagtttcttcattcatc

SEQ ID NO: 314 - DNA, 20 bp, Homo sapiens
agatagtttcgtcattcatc

SEQ ID NO: 315 - DNA, 20 bp, Homo sapiens
agatagtttcgttattcatc

SEQ ID NO: 316 - DNA, 21 bp, Homo sapiens
ccacccctcatcatacctcaa

SEQ ID NO: 317 - DNA, 21 bp, Homo sapiens
cacccctcattatacctcaac

SEQ ID NO: 318 - DNA, 21 bp, Homo sapiens
acccctcatcttacctcaacc

SEQ ID NO: 319 - DNA, 21 bp, Homo sapiens
cccctcatcatacctcaacca

SEQ ID NO: 320 - DNA, 21 bp, Homo sapiens
ccctcatcattcctcaaccac

SEQ ID NO: 321 - DNA, 21 bp, Homo sapiens
cctcatcatatctcaaccacc

SEQ ID NO: 322 - DNA, 21 bp, Homo sapiens
ctcatcatacttcaaccacca

SEQ ID NO: 323 - DNA, 21 bp, Homo sapiens
tcatcatacctcaaccaccac

SEQ ID NO: 324 - DNA, 21 bp, Homo sapiens
catcataccttaaccaccacc

SEQ ID NO: 325 - DNA, 21 bp, Homo sapiens
ccctctcatcgatcacccacc

SEQ ID NO: 326 - DNA, 21 bp, Homo sapiens
cctctcatccgtcacccacca

SEQ ID NO: 327 - DNA, 21 bp, Homo sapiens
ctctcatccagcacccaccac

SEQ ID NO: 328 - DNA, 21 bp, Homo sapiens
tctcatccatgacccaccacc

SEQ ID NO: 329 - DNA, 21 bp, Homo sapiens
ctcatccatcgcccaccaccc

SEQ ID NO: 330 - DNA, 21 bp, Homo sapiens
tcatccatcagccaccacccc

SEQ ID NO: 331 - DNA, 21 bp, Homo sapiens
catccatcacgcaccacccct

SEQ ID NO: 332 - DNA, 21 bp, Homo sapiens
atccatcaccgaccacccctc

SEQ ID NO: 333 - DNA, 21 bp, Homo sapiens
tccatcacccgccacccctca

SEQ ID NO: 334 - DNA, 21 bp, Homo sapiens
ccatcacccagcacccctcat

SEQ ID NO: 335 - DNA, 21 bp, Homo sapiens
catcacccacgacccctcatc

SEQ ID NO: 336 - DNA, 21 bp, Homo sapiens
atcacccaccgcccctcatca

SEQ ID NO: 337 - DNA, 21 bp, Homo sapiens
tcacccaccagccctcatcat

SEQ ID NO: 338 - DNA, 21 bp, Homo sapiens
cacccaccacgcctcatcata

SEQ ID NO: 339 - DNA, 21 bp, Homo sapiens
acccaccaccgctcatcatac

SEQ ID NO: 340 - DNA, 21 bp, Homo sapiens
cccaccacccgtcatcatacc

SEQ ID NO: 341 - DNA, 21 bp, Homo sapiens
ccaccaccccgcatcatacct

SEQ ID NO: 342 - DNA, 21 bp, Homo sapiens
caccacccctgatcatacctc

SEQ ID NO: 343 - DNA, 21 bp, Homo sapiens
accacccctcgtcatacctca

SEQ ID NO: 344 - DNA, 20 bp, Homo sapiens
tacattgcccatgtaattaa

SEQ ID NO: 345 - DNA, 20 bp, Homo sapiens
atatagtttcgtcattcatc

SEQ ID NO: 346 - DNA, 20 bp, Homo sapiens
tacattgcccatgtaattaa

SEQ ID NO: 347 - DNA, 20 bp, Homo sapiens
atatagtttcgtcattcatc

SEQ ID NO: 348 - DNA, 20 bp, Homo sapiens
agatagtttggtcattcatc

SEQ ID NO: 349 - DNA, 20 bp, Homo sapiens
agatagtttcgtcattcatc

SEQ ID NO: 350 - DNA, 20 bp, Homo sapiens
agatagtttcggcattcatc

SEQ ID NO: 351 - DNA, 20 bp, Homo sapiens
agatagtttcgtgattcatc

SEQ ID NO: 352 - DNA, 21 bp, Homo sapiens
ccacccctcagcatacctcaa

SEQ ID NO: 353 - DNA, 21 bp, Homo sapiens
cacccctcatgatacctcaac

SEQ ID NO: 354 - DNA, 21 bp, Homo sapiens
acccctcatcgtacctcaacc

SEQ ID NO: 355 - DNA, 21 bp, Homo sapiens
cccctcatcagacctcaacca

SEQ ID NO: 356 - DNA, 21 bp, Homo sapiens
ccctcatcatgcctcaaccac

SEQ ID NO: 357 - DNA, 21 bp, Homo sapiens
cctcatcatagctcaaccacc

SEQ ID NO: 358 - DNA, 21 bp, Homo sapiens
ctcatcatacgtcaaccacca

SEQ ID NO: 359 - DNA, 21 bp, Homo sapiens
tcatcataccgcaaccaccac

SEQ ID NO: 360 - DNA, 21 bp, Homo sapiens
catcatacctgaaccaccacc

SEQ ID NO: 361 - DNA, 21 bp, Homo sapiens
ccctctcatccatcacccacc

SEQ ID NO: 362 - DNA, 21 bp, Homo sapiens
cctctcatccctcacccacca

SEQ ID NO: 363 - DNA, 21 bp, Homo sapiens
ctctcatccaccacccaccac

SEQ ID NO: 364 - DNA, 21 bp, Homo sapiens
tctcatccatcacccaccacc

SEQ ID NO: 365 - DNA, 21 bp, Homo sapiens
ctcatccatcccccaccaccc

SEQ ID NO: 366 - DNA, 21 bp, Homo sapiens
tcatccatcacccaccacccc

SEQ ID NO: 367 - DNA, 21 bp, Homo sapiens
catccatcacccaccacccct

SEQ ID NO: 368 - DNA, 21 bp, Homo sapiens
atccatcacccaccacccctc

SEQ ID NO: 369 - DNA, 21 bp, Homo sapiens
tccatcaccccccacccctca

SEQ ID NO: 370 - DNA, 21 bp, Homo sapiens
ccatcacccaccacccctcat

SEQ ID NO: 371 - DNA, 21 bp, Homo sapiens
catcacccaccacccctcatc

SEQ ID NO: 372 - DNA, 21 bp, Homo sapiens
atcacccaccccccctcatca

SEQ ID NO: 373 - DNA, 21 bp, Homo sapiens
tcacccaccacccctcatcat

SEQ ID NO: 374 - DNA, 21 bp, Homo sapiens
cacccaccacccctcatcata

SEQ ID NO: 375 - DNA, 21 bp, Homo sapiens
acccaccacccctcatcatac

SEQ ID NO: 376 - DNA, 21 bp, Homo sapiens
cccaccacccctcatcatacc

SEQ ID NO: 377 - DNA, 21 bp, Homo sapiens
ccaccaccccccatcatacct

SEQ ID NO: 378 - DNA, 21 bp, Homo sapiens
caccacccctcatcatacctc

SEQ ID NO: 379 - DNA, 21 bp, Homo sapiens
accacccctcctcatacctca

SEQ ID NO: 380 - DNA, 20 bp, Homo sapiens
atatagtttcgtcattcatc

SEQ ID NO: 381 - DNA, 20 bp, Homo sapiens
tacattgcccatgtaattaa

SEQ ID NO: 382 - DNA, 20 bp, Homo sapiens
atatagtttcgtcattcatc

SEQ ID NO: 383 - DNA, 20 bp, Homo sapiens
tacattgcccatgtaattaa

SEQ ID NO: 384 - DNA, 20 bp, Homo sapiens
agatagtttcgtcattcatc

SEQ ID NO: 385 - DNA, 20 bp, Homo sapiens
agatagtttcctcattcatc

SEQ ID NO: 386 - DNA, 20 bp, Homo sapiens
agatagtttcgccattcatc

SEQ ID NO: 387 - DNA, 20 bp, Homo sapiens
agatagtttcgtcattcatc

SEQ ID NO: 388 - DNA, 21 bp, Homo sapiens
ccacccctcaccatacctcaa

SEQ ID NO: 389 - DNA, 21 bp, Homo sapiens
cacccctcatcatacctcaac

SEQ ID NO: 390 - DNA, 21 bp, Homo sapiens
acccctcatcctacctcaacc

SEQ ID NO: 391 - DNA, 21 bp, Homo sapiens
cccctcatcacacctcaacca

SEQ ID NO: 392 - DNA, 21 bp, Homo sapiens
ccctcatcatccctcaaccac

SEQ ID NO: 393 - DNA, 21 bp, Homo sapiens
cctcatcatacctcaaccacc

SEQ ID NO: 394 - DNA, 21 bp, Homo sapiens
ctcatcatacctcaaccacca

SEQ ID NO: 395 - DNA, 21 bp, Homo sapiens
tcatcataccccaaccaccac

SEQ ID NO: 396 - DNA, 21 bp, Homo sapiens
catcatacctcaaccaccacc

SEQ ID NO: 397 - DNA, 21 bp, Homo sapiens
ccctctcatcaatcacccacc

SEQ ID NO: 398 - DNA, 21 bp, Homo sapiens
cctctcatccatcacccacca

SEQ ID NO: 399 - DNA, 21 bp, Homo sapiens
ctctcatccaacacccaccac

SEQ ID NO: 400 - DNA, 21 bp, Homo sapiens
tctcatccataacccaccacc

SEQ ID NO: 401 - DNA, 21 bp, Homo sapiens
ctcatccatcacccaccaccc

SEQ ID NO: 402 - DNA, 21 bp, Homo sapiens
tcatccatcaaccaccacccc

SEQ ID NO: 403 - DNA, 21 bp, Homo sapiens
catccatcacacaccacccct

SEQ ID NO: 404 - DNA, 21 bp, Homo sapiens
atccatcaccaaccacccctc

SEQ ID NO: 405 - DNA, 21 bp, Homo sapiens
tccatcacccaccacccctca

SEQ ID NO: 406 - DNA, 21 bp, Homo sapiens
ccatcacccaacacccctcat

SEQ ID NO: 407 - DNA, 21 bp, Homo sapiens
catcacccacaacccctcatc

SEQ ID NO: 408 - DNA, 21 bp, Homo sapiens
atcacccaccacccctcatca

SEQ ID NO: 409 - DNA, 21 bp, Homo sapiens
tcacccaccaaccctcatcat

SEQ ID NO: 410 - DNA, 21 bp, Homo sapiens
cacccaccacacctcatcata

SEQ ID NO: 411 - DNA, 21 bp, Homo sapiens
acccaccaccactcatcatac

SEQ ID NO: 412 - DNA, 21 bp, Homo sapiens
cccaccacccatcatcatacc

SEQ ID NO: 413 - DNA, 21 bp, Homo sapiens
ccaccaccccacatcatacct

SEQ ID NO: 414 - DNA, 21 bp, Homo sapiens
caccacccctaatcatacctc

SEQ ID NO: 415 - DNA, 21 bp, Homo sapiens
accacccctcatcatacctca

SEQ ID NO: 416 - DNA, 20 bp, Homo sapiens
tacattgcccatgtaattaa

SEQ ID NO: 417 - DNA, 20 bp, Homo sapiens
atatagtttcgtcattcatc

SEQ ID NO: 418 - DNA, 20 bp, Homo sapiens
tacattgcccatgtaattaa

SEQ ID NO: 419 - DNA, 20 bp, Homo sapiens
atatagtttcgtcattcatc

SEQ ID NO: 420 - DNA, 20 bp, Homo sapiens
agatagtttagtcattcatc

SEQ ID NO: 421 - DNA, 20 bp, Homo sapiens
agatagtttcatcattcatc

SEQ ID NO: 422 - DNA, 20 bp, Homo sapiens
agatagtttcgacattcatc

SEQ ID NO: 423 - DNA, 20 bp, Homo sapiens
agatagtttcgtaattcatc

SEQ ID NO: 424 - DNA, 21 bp, Homo sapiens
ccacccctcaacatacctcaa

SEQ ID NO: 425 - DNA, 21 bp, Homo sapiens
cacccctcataatacctcaac

SEQ ID NO: 426 - DNA, 21 bp, Homo sapiens
acccctcatcatacctcaacc

SEQ ID NO: 427 - DNA, 21 bp, Homo sapiens
cccctcatcaaacctcaacca

SEQ ID NO: 428 - DNA, 21 bp, Homo sapiens
ccctcatcatacctcaaccac

SEQ ID NO: 429 - DNA, 21 bp, Homo sapiens
cctcatcataactcaaccacc

SEQ ID NO: 430 - DNA, 21 bp, Homo sapiens
ctcatcatacatcaaccacca

SEQ ID NO: 431 - DNA, 21 bp, Homo sapiens
tcatcataccacaaccaccac

SEQ ID NO: 432 - DNA, 21 bp, Homo sapiens
catcatacctaaaccaccacc

SEQ ID NO: 433 - DNA, 21 bp, Homo sapiens
atcatacctctaccaccaccc

SEQ ID NO: 434 - DNA, 21 bp, Homo sapiens
tcatacctcatccaccacccc

SEQ ID NO: 435 - DNA, 21 bp, Homo sapiens
catacctcaatcaccacccct

SEQ ID NO: 436 - DNA, 21 bp, Homo sapiens
atacctcaactaccacccctc

SEQ ID NO: 437 - DNA, 21 bp, Homo sapiens
tacctcaacctccacccctca

SEQ ID NO: 438 - DNA, 21 bp, Homo sapiens
acctcaaccatcacccctcat

SEQ ID NO: 439 - DNA, 21 bp, Homo sapiens
cctcaaccactacccctcatc

SEQ ID NO: 440 - DNA, 21 bp, Homo sapiens
ctcaaccacctcccctcatca

SEQ ID NO: 441 - DNA, 21 bp, Homo sapiens
tcaaccaccatccctcatcat

SEQ ID NO: 442 - DNA, 21 bp, Homo sapiens
caaccaccactcctcatcata

SEQ ID NO: 443 - DNA, 21 bp, Homo sapiens
aaccaccacctctcatcatac

SEQ ID NO: 444 - DNA, 21 bp, Homo sapiens
accaccacccttcatcatacc

SEQ ID NO: 445 - DNA, 21 bp, Homo sapiens
ccaccacccctcatcatacct

SEQ ID NO: 446 - DNA, 21 bp, Homo sapiens
caccaccccttatcatacctc

SEQ ID NO: 447 - DNA, 21 bp, Homo sapiens
accacccctcttcatacctca

SEQ ID NO: 448 - DNA, 21 bp, Homo sapiens
ccacccctcatcatacctcaa

SEQ ID NO: 449 - DNA, 20 bp, Homo sapiens
atatagtttcgtcattcatc

SEQ ID NO: 450 - DNA, 20 bp, Homo sapiens
tacattgcccatgtaattaa

SEQ ID NO: 451 - DNA, 20 bp, Homo sapiens
atatagtttcgtcattcatc

SEQ ID NO: 452 - DNA, 20 bp, Homo sapiens
tacattgcccatgtaattaa

SEQ ID NO: 453 - DNA, 20 bp, Homo sapiens
agatagttttgtcattcatc

SEQ ID NO: 454 - DNA, 20 bp, Homo sapiens
agatagtttcttcattcatc

SEQ ID NO: 455 - DNA, 20 bp, Homo sapiens
agatagtttcgtcattcatc

SEQ ID NO: 456 - DNA, 20 bp, Homo sapiens
agatagtttcgttattcatc

SEQ ID NO: 457 - DNA, 21 bp, Homo sapiens
cacccctcattatacctcaaa

SEQ ID NO: 458 - DNA, 21 bp, Homo sapiens
acccctcatcttacctcaaaa

SEQ ID NO: 459 - DNA, 21 bp, Homo sapiens
cccctcatcatacctcaaaaa

SEQ ID NO: 460 - DNA, 21 bp, Homo sapiens
ccctcatcattcctcaaaaac

SEQ ID NO: 461 - DNA, 21 bp, Homo sapiens
cctcatcatatctcaaaaacc

SEQ ID NO: 462 - DNA, 21 bp, Homo sapiens
ctcatcatacttcaaaaacca

SEQ ID NO: 463 - DNA, 21 bp, Homo sapiens
tcatcatacctcaaaaaccaa

SEQ ID NO: 464 - DNA, 21 bp, Homo sapiens
catcataccttaaaaaccaac

SEQ ID NO: 465 - DNA, 21 bp, Homo sapiens
atcatacctctaaaaccaact

SEQ ID NO: 466 - DNA, 21 bp, Homo sapiens
tcatacctcataaaccaacta

SEQ ID NO: 467 - DNA, 21 bp, Homo sapiens
catacctcaataaccaactaa

SEQ ID NO: 468 - DNA, 21 bp, Homo sapiens
atacctcaaataccaactaac

SEQ ID NO: 469 - DNA, 21 bp, Homo sapiens
atcatacctcgaccaccaccc

SEQ ID NO: 470 - DNA, 21 bp, Homo sapiens
tcatacctcagccaccacccc

SEQ ID NO: 471 - DNA, 21 bp, Homo sapiens
catacctcaagcaccacccct

SEQ ID NO: 472 - DNA, 21 bp, Homo sapiens
atacctcaacgaccacccctc

SEQ ID NO: 473 - DNA, 21 bp, Homo sapiens
tacctcaaccgccacccctca

SEQ ID NO: 474 - DNA, 21 bp, Homo sapiens
acctcaaccagcacccctcat

SEQ ID NO: 475 - DNA, 21 bp, Homo sapiens
cctcaaccacgacccctcatc

SEQ ID NO: 476 - DNA, 21 bp, Homo sapiens
ctcaaccaccgcccctcatca

SEQ ID NO: 477 - DNA, 21 bp, Homo sapiens
tcaaccaccagccctcatcat

SEQ ID NO: 478 - DNA, 21 bp, Homo sapiens
caaccaccacgcctcatcata

SEQ ID NO: 479 - DNA, 21 bp, Homo sapiens
aaccaccaccgctcatcatac

SEQ ID NO: 480 - DNA, 21 bp, Homo sapiens
accaccacccgtcatcatacc

SEQ ID NO: 481 - DNA, 21 bp, Homo sapiens
ccaccaccccgcatcatacct

SEQ ID NO: 482 - DNA, 21 bp, Homo sapiens
caccacccctgatcatacctc

SEQ ID NO: 483 - DNA, 21 bp, Homo sapiens
accacccctcgtcatacctca

SEQ ID NO: 484 - DNA, 21 bp, Homo sapiens
ccacccctcagcatacctcaa

SEQ ID NO: 485 - DNA, 20 bp, Homo sapiens
tacattgcccatgtaattaa

SEQ ID NO: 486 - DNA, 20 bp, Homo sapiens
atatagtttcgtcattcatc

SEQ ID NO: 487 - DNA, 20 bp, Homo sapiens
tacattgcccatgtaattaa

SEQ ID NO: 488 - DNA, 20 bp, Homo sapiens
atatagtttcgtcattcatc

SEQ ID NO: 489 - DNA, 20 bp, Homo sapiens
agatagtttggtcattcatc

SEQ ID NO: 490 - DNA, 20 bp, Homo sapiens
agatagtttcgtcattcatc

SEQ ID NO: 491 - DNA, 20 bp, Homo sapiens
agatagtttcggcattcatc

SEQ ID NO: 492 - DNA, 20 bp, Homo sapiens
agatagtttcgtgattcatc

SEQ ID NO: 493 - DNA, 21 bp, Homo sapiens
cacccctcatgatacctcaaa

SEQ ID NO: 494 - DNA, 21 bp, Homo sapiens
acccctcatcgtacctcaaaa

SEQ ID NO: 495 - DNA, 21 bp, Homo sapiens
cccctcatcagacctcaaaaa

SEQ ID NO: 496 - DNA, 21 bp, Homo sapiens
ccctcatcatgcctcaaaaac

SEQ ID NO: 497 - DNA, 21 bp, Homo sapiens
cctcatcatagctcaaaaacc

SEQ ID NO: 498 - DNA, 21 bp, Homo sapiens
ctcatcatacgtcaaaaacca

SEQ ID NO: 499 - DNA, 21 bp, Homo sapiens
tcatcataccgcaaaaaccaa

SEQ ID NO: 500 - DNA, 21 bp, Homo sapiens
catcatacctgaaaaaccaac

SEQ ID NO: 501 - DNA, 21 bp, Homo sapiens
atcatacctcgaaaaccaact

SEQ ID NO: 502 - DNA, 21 bp, Homo sapiens
tcatacctcagaaaccaacta

SEQ ID NO: 503 - DNA, 21 bp, Homo sapiens
catacctcaagaaccaactaa

SEQ ID NO: 504 - DNA, 21 bp, Homo sapiens
atacctcaaagaccaactaac

SEQ ID NO: 505 - DNA, 21 bp, Homo sapiens
atcatacctccaccaccaccc

SEQ ID NO: 506 - DNA, 21 bp, Homo sapiens
tcatacctcacccaccacccc

SEQ ID NO: 507 - DNA, 21 bp, Homo sapiens
catacctcaaccaccacccct

SEQ ID NO: 508 - DNA, 21 bp, Homo sapiens
atacctcaaccaccacccctc

SEQ ID NO: 509 - DNA, 21 bp, Homo sapiens
tacctcaacccccacccctca

SEQ ID NO: 510 - DNA, 21 bp, Homo sapiens
acctcaaccaccacccctcat

SEQ ID NO: 511 - DNA, 21 bp, Homo sapiens
cctcaaccaccacccctcatc

SEQ ID NO: 512 - DNA, 21 bp, Homo sapiens
ctcaaccaccccccctcatca

SEQ ID NO: 513 - DNA, 21 bp, Homo sapiens
tcaaccaccacccctcatcat

SEQ ID NO: 514 - DNA, 21 bp, Homo sapiens
caaccaccacccctcatcata

SEQ ID NO: 515 - DNA, 21 bp, Homo sapiens
aaccaccacccctcatcatac

SEQ ID NO: 516 - DNA, 21 bp, Homo sapiens
accaccacccctcatcatacc

SEQ ID NO: 517 - DNA, 21 bp, Homo sapiens
ccaccaccccccatcatacct

SEQ ID NO: 518 - DNA, 21 bp, Homo sapiens
caccacccctcatcatacctc

SEQ ID NO: 519 - DNA, 21 bp, Homo sapiens
accacccctcctcatacctca

SEQ ID NO: 520 - DNA, 21 bp, Homo sapiens
ccacccctcaccatacctcaa

SEQ ID NO: 521 - DNA, 20 bp, Homo sapiens
atatagtttcgtcattcatc

SEQ ID NO: 522 - DNA, 20 bp, Homo sapiens
tacattgcccatgtaattaa

SEQ ID NO: 523 - DNA, 20 bp, Homo sapiens
atatagtttcgtcattcatc

SEQ ID NO: 524 - DNA, 20 bp, Homo sapiens
tacattgcccatgtaattaa

SEQ ID NO: 525 - DNA, 20 bp, Homo sapiens
agatagtttcgtcattcatc

SEQ ID NO: 526 - DNA, 20 bp, Homo sapiens
agatagtttcctcattcatc

SEQ ID NO: 527 - DNA, 20 bp, Homo sapiens
agatagtttcgccattcatc

SEQ ID NO: 528 - DNA, 20 bp, Homo sapiens
agatagtttcgtcattcatc

SEQ ID NO: 529 - DNA, 21 bp, Homo sapiens
cacccctcatcatacctcaaa

SEQ ID NO: 530 - DNA, 21 bp, Homo sapiens
acccctcatcctacctcaaaa

SEQ ID NO: 531 - DNA, 21 bp, Homo sapiens
cccctcatcacacctcaaaaa

SEQ ID NO: 532 - DNA, 21 bp, Homo sapiens
ccctcatcatccctcaaaaac

SEQ ID NO: 533 - DNA, 21 bp, Homo sapiens
cctcatcatacctcaaaaacc

SEQ ID NO: 534 - DNA, 21 bp, Homo sapiens
ctcatcatacctcaaaaacca

SEQ ID NO: 535 - DNA, 21 bp, Homo sapiens
tcatcataccccaaaaaccaa

SEQ ID NO: 536 - DNA, 21 bp, Homo sapiens
catcatacctcaaaaaccaac

SEQ ID NO: 537 - DNA, 21 bp, Homo sapiens
atcatacctccaaaaccaact

SEQ ID NO: 538 - DNA, 21 bp, Homo sapiens
tcatacctcacaaaccaacta

SEQ ID NO: 539 - DNA, 21 bp, Homo sapiens
catacctcaacaaccaactaa

SEQ ID NO: 540 - DNA, 21 bp, Homo sapiens
atacctcaaacaccaactaac

SEQ ID NO: 541 - DNA, 21 bp, Homo sapiens
atcatacctcaaccaccaccc

SEQ ID NO: 542 - DNA, 21 bp, Homo sapiens
tcatacctcaaccaccacccc

SEQ ID NO: 543 - DNA, 21 bp, Homo sapiens
catacctcaaacaccacccct

SEQ ID NO: 544 - DNA, 21 bp, Homo sapiens
atacctcaacaaccacccctc

SEQ ID NO: 545 - DNA, 21 bp, Homo sapiens
tacctcaaccaccacccctca

SEQ ID NO: 546 - DNA, 21 bp, Homo sapiens
acctcaaccaacacccctcat

SEQ ID NO: 547 - DNA, 21 bp, Homo sapiens
cctcaaccacaacccctcatc

SEQ ID NO: 548 - DNA, 21 bp, Homo sapiens
ctcaaccaccacccctcatca

SEQ ID NO: 549 - DNA, 21 bp, Homo sapiens
tcaaccaccaaccctcatcat

SEQ ID NO: 550 - DNA, 21 bp, Homo sapiens
caaccaccacacctcatcata

SEQ ID NO: 551 - DNA, 21 bp, Homo sapiens
aaccaccaccactcatcatac

SEQ ID NO: 552 - DNA, 21 bp, Homo sapiens
accaccacccatcatcatacc

SEQ ID NO: 553 - DNA, 21 bp, Homo sapiens
ccaccaccccacatcatacct

SEQ ID NO: 554 - DNA, 21 bp, Homo sapiens
caccacccctaatcatacctc

SEQ ID NO: 555 - DNA, 21 bp, Homo sapiens
accacccctcatcatacctca

SEQ ID NO: 556 - DNA, 21 bp, Homo sapiens
ccacccctcaacatacctcaa

SEQ ID NO: 557 - DNA, 20 bp, Homo sapiens
tacattgcccatgtaattaa

SEQ ID NO: 558 - DNA, 20 bp, Homo sapiens
atatagtttcgtcattcatc

SEQ ID NO: 559 - DNA, 20 bp, Homo sapiens
tacattgcccatgtaattaa

SEQ ID NO: 560 - DNA, 20 bp, Homo sapiens
atatagtttcgtcattcatc

SEQ ID NO: 561 - DNA, 20 bp, Homo sapiens
agatagtttagtcattcatc

SEQ ID NO: 562 - DNA, 20 bp, Homo sapiens
agatagtttcatcattcatc

SEQ ID NO: 563 - DNA, 20 bp, Homo sapiens
agatagtttcgacattcatc

SEQ ID NO: 564 - DNA, 20 bp, Homo sapiens
agatagtttcgtaattcatc

SEQ ID NO: 565 - DNA, 21 bp, Homo sapiens
cacccctcataatacctcaaa

SEQ ID NO: 566 - DNA, 21 bp, Homo sapiens
acccctcatcatacctcaaaa

SEQ ID NO: 567 - DNA, 21 bp, Homo sapiens
cccctcatcaaacctcaaaaa

SEQ ID NO: 568 - DNA, 21 bp, Homo sapiens
ccctcatcatacctcaaaaac

SEQ ID NO: 569 - DNA, 21 bp, Homo sapiens
cctcatcataactcaaaaacc

SEQ ID NO: 570 - DNA, 21 bp, Homo sapiens
ctcatcatacatcaaaaacca

SEQ ID NO: 571 - DNA, 21 bp, Homo sapiens
tcatcataccacaaaaaccaa

SEQ ID NO: 572 - DNA, 21 bp, Homo sapiens
catcatacctaaaaaaccaac

SEQ ID NO: 573 - DNA, 21 bp, Homo sapiens
atcatacctcaaaaaccaact

SEQ ID NO: 574 - DNA, 21 bp, Homo sapiens
tcatacctcaaaaaccaacta

SEQ ID NO: 575 - DNA, 21 bp, Homo sapiens
catacctcaaaaaccaactaa

SEQ ID NO: 576 - DNA, 21 bp, Homo sapiens
atacctcaaaaaccaactaac

SEQ ID NO: 577 - DNA, 21 bp, Homo sapiens
tacctcaaaatccaactaacc

SEQ ID NO: 578 - DNA, 21 bp, Homo sapiens
acctcaaaaatcaactaacca

SEQ ID NO: 579 - DNA, 21 bp, Homo sapiens
cctcaaaaactaactaaccaa

SEQ ID NO: 580 - DNA, 21 bp, Homo sapiens
ctcaaaaacctactaaccaac

SEQ ID NO: 581 - DNA, 21 bp, Homo sapiens
tcaaaaaccatctaaccaacc

SEQ ID NO: 582 - DNA, 21 bp, Homo sapiens
caaaaaccaattaaccaacca

SEQ ID NO: 583 - DNA, 21 bp, Homo sapiens
aaaaaccaactaaccaaccaa

SEQ ID NO: 584 - DNA, 21 bp, Homo sapiens
aaaaccaacttaccaaccaat

SEQ ID NO: 585 - DNA, 21 bp, Homo sapiens
aaccaaccaataatctcccac

SEQ ID NO: 586 - DNA, 21 bp, Homo sapiens
accaaccaattatctcccacc

SEQ ID NO: 587 - DNA, 21 bp, Homo sapiens
ccaaccaatattctcccaccc

SEQ ID NO: 588 - DNA, 21 bp, Homo sapiens
caaccaataatctcccacccc

SEQ ID NO: 589 - DNA, 21 bp, Homo sapiens
aaccaataatttcccaccccg

SEQ ID NO: 590 - DNA, 20 bp, Homo sapiens
atatagtttcgtcattcatc

SEQ ID NO: 591 - DNA, 20 bp, Homo sapiens
tacattgcccatgtaattaa

SEQ ID NO: 592 - DNA, 20 bp, Homo sapiens
atatagtttcgtcattcatc

SEQ ID NO: 593 - DNA, 20 bp, Homo sapiens
tacattgcccatgtaattaa

SEQ ID NO: 594 - DNA, 20 bp, Homo sapiens
agatagttttgtcattcatc

SEQ ID NO: 595 - DNA, 20 bp, Homo sapiens
agatagtttcttcattcatc

SEQ ID NO: 596 - DNA, 20 bp, Homo sapiens
agatagtttcgtcattcatc

SEQ ID NO: 597 - DNA, 20 bp, Homo sapiens
agatagtttcgttattcatc

SEQ ID NO: 598 - DNA, 21 bp, Homo sapiens
accaataatctcccaccccgc

SEQ ID NO: 599 - DNA, 21 bp, Homo sapiens
ccaataatcttccaccccgcc

SEQ ID NO: 600 - DNA, 21 bp, Homo sapiens
caataatctctcaccccgcct

SEQ ID NO: 601 - DNA, 21 bp, Homo sapiens
aataatctcctaccccgccta

SEQ ID NO: 602 - DNA, 21 bp, Homo sapiens
ataatctccctccccgcctag

SEQ ID NO: 603 - DNA, 21 bp, Homo sapiens
taatctcccatcccgcctagc

SEQ ID NO: 604 - DNA, 21 bp, Homo sapiens
aatctcccactccgcctagct

SEQ ID NO: 605 - DNA, 21 bp, Homo sapiens
atctcccacctcgcctagctc

SEQ ID NO: 606 - DNA, 21 bp, Homo sapiens
tctcccaccctgcctagctca

SEQ ID NO: 607 - DNA, 21 bp, Homo sapiens
ctcccacccctcctagctcac

SEQ ID NO: 608 - DNA, 21 bp, Homo sapiens
tcccaccccgtctagctcacg

SEQ ID NO: 609 - DNA, 21 bp, Homo sapiens
cccaccccgcttagctcacgc

SEQ ID NO: 610 - DNA, 21 bp, Homo sapiens
ccaccccgcctagctcacgca

SEQ ID NO: 611 - DNA, 21 bp, Homo sapiens
caccccgccttgctcacgcaa

SEQ ID NO: 612 - DNA, 21 bp, Homo sapiens
accccgcctatctcacgcaag

SEQ ID NO: 613 - DNA, 21 bp, Homo sapiens
tacctcaaaagccaactaacc

SEQ ID NO: 614 - DNA, 21 bp, Homo sapiens
acctcaaaaagcaactaacca

SEQ ID NO: 615 - DNA, 21 bp, Homo sapiens
cctcaaaaacgaactaaccaa

SEQ ID NO: 616 - DNA, 21 bp, Homo sapiens
ctcaaaaaccgactaaccaac

SEQ ID NO: 617 - DNA, 21 bp, Homo sapiens
tcaaaaaccagctaaccaacc

SEQ ID NO: 618 - DNA, 21 bp, Homo sapiens
caaaaaccaagtaaccaacca

SEQ ID NO: 619 - DNA, 21 bp, Homo sapiens
aaaaaccaacgaaccaaccaa

SEQ ID NO: 620 - DNA, 21 bp, Homo sapiens
aaaaccaactgaccaaccaat

SEQ ID NO: 621 - DNA, 21 bp, Homo sapiens
aaccaaccaagaatctcccac

SEQ ID NO: 622 - DNA, 21 bp, Homo sapiens
accaaccaatgatctcccacc

SEQ ID NO: 623 - DNA, 21 bp, Homo sapiens
ccaaccaatagtctcccaccc

SEQ ID NO: 624 - DNA, 21 bp, Homo sapiens
caaccaataagctcccacccc

SEQ ID NO: 625 - DNA, 21 bp, Homo sapiens
aaccaataatgtcccaccccg

SEQ ID NO: 626 - DNA, 20 bp, Homo sapiens
tacattgcccatgtaattaa

SEQ ID NO: 627 - DNA, 20 bp, Homo sapiens
atatagtttcgtcattcatc

SEQ ID NO: 628 - DNA, 20 bp, Homo sapiens
tacattgcccatgtaattaa

SEQ ID NO: 629 - DNA, 20 bp, Homo sapiens
atatagtttcgtcattcatc

SEQ ID NO: 630 - DNA, 20 bp, Homo sapiens
agatagtttggtcattcatc

SEQ ID NO: 631 - DNA, 20 bp, Homo sapiens
agatagtttcgtcattcatc

SEQ ID NO: 632 - DNA, 20 bp, Homo sapiens
agatagtttcggcattcatc

SEQ ID NO: 633 - DNA, 20 bp, Homo sapiens
agatagtttcgtgattcatc

SEQ ID NO: 634 - DNA, 21 bp, Homo sapiens
accaataatcgcccaccccgc

SEQ ID NO: 635 - DNA, 21 bp, Homo sapiens
ccaataatctgccaccccgcc

SEQ ID NO: 636 - DNA, 21 bp, Homo sapiens
caataatctcgcaccccgcct

SEQ ID NO: 637 - DNA, 21 bp, Homo sapiens
aataatctccgaccccgccta

SEQ ID NO: 638 - DNA, 21 bp, Homo sapiens
ataatctcccgccccgcctag

SEQ ID NO: 639 - DNA, 21 bp, Homo sapiens
taatctcccagcccgcctagc

SEQ ID NO: 640 - DNA, 21 bp, Homo sapiens
aatctcccacgccgcctagct

SEQ ID NO: 641 - DNA, 21 bp, Homo sapiens
atctcccaccgcgcctagctc

SEQ ID NO: 642 - DNA, 21 bp, Homo sapiens
tctcccacccggcctagctca

SEQ ID NO: 643 - DNA, 21 bp, Homo sapiens
ctcccaccccgcctagctcac

SEQ ID NO: 644 - DNA, 21 bp, Homo sapiens
tcccaccccggctagctcacg

SEQ ID NO: 645 - DNA, 21 bp, Homo sapiens
cccaccccgcgtagctcacgc

SEQ ID NO: 646 - DNA, 21 bp, Homo sapiens
ccaccccgccgagctcacgca

SEQ ID NO: 647 - DNA, 21 bp, Homo sapiens
caccccgcctggctcacgcaa

SEQ ID NO: 648 - DNA, 21 bp, Homo sapiens
accccgcctagctcacgcaag

SEQ ID NO: 649 - DNA, 21 bp, Homo sapiens
tacctcaaaacccaactaacc

SEQ ID NO: 650 - DNA, 21 bp, Homo sapiens
acctcaaaaaccaactaacca

SEQ ID NO: 651 - DNA, 21 bp, Homo sapiens
cctcaaaaaccaactaaccaa

SEQ ID NO: 652 - DNA, 21 bp, Homo sapiens
ctcaaaaacccactaaccaac

SEQ ID NO: 653 - DNA, 21 bp, Homo sapiens
tcaaaaaccacctaaccaacc

SEQ ID NO: 654 - DNA, 21 bp, Homo sapiens
caaaaaccaactaaccaacca

SEQ ID NO: 655 - DNA, 21 bp, Homo sapiens
aaaaaccaaccaaccaaccaa

SEQ ID NO: 656 - DNA, 21 bp, Homo sapiens
aaaaccaactcaccaaccaat

SEQ ID NO: 657 - DNA, 21 bp, Homo sapiens
aaccaaccaacaatctcccac

SEQ ID NO: 658 - DNA, 21 bp, Homo sapiens
accaaccaatcatctcccacc

SEQ ID NO: 659 - DNA, 21 bp, Homo sapiens
ccaaccaatactctcccaccc

SEQ ID NO: 660 - DNA, 21 bp, Homo sapiens
caaccaataacctcccacccc

SEQ ID NO: 661 - DNA, 21 bp, Homo sapiens
aaccaataatctcccaccccg

SEQ ID NO: 662 - DNA, 20 bp, Homo sapiens
atatagtttcgtcattcatc

SEQ ID NO: 663 - DNA, 20 bp, Homo sapiens
tacattgcccatgtaattaa

SEQ ID NO: 664 - DNA, 20 bp, Homo sapiens
atatagtttcgtcattcatc

SEQ ID NO: 665 - DNA, 20 bp, Homo sapiens
tacattgcccatgtaattaa

SEQ ID NO: 666 - DNA, 20 bp, Homo sapiens
agatagtttcgtcattcatc

SEQ ID NO: 667 - DNA, 20 bp, Homo sapiens
agatagtttcctcattcatc

SEQ ID NO: 668 - DNA, 20 bp, Homo sapiens
agatagtttcgccattcatc

SEQ ID NO: 669 - DNA, 20 bp, Homo sapiens
agatagtttcgtcattcatc

SEQ ID NO: 670 - DNA, 21 bp, Homo sapiens
accaataatcccccaccccgc

SEQ ID NO: 671 - DNA, 21 bp, Homo sapiens
ccaataatctcccaccccgcc

SEQ ID NO: 672 - DNA, 21 bp, Homo sapiens
caataatctcccaccccgcct

SEQ ID NO: 673 - DNA, 21 bp, Homo sapiens
aataatctcccaccccgccta

SEQ ID NO: 674 - DNA, 21 bp, Homo sapiens
ataatctccccccccgcctag

SEQ ID NO: 675 - DNA, 21 bp, Homo sapiens
taatctcccaccccgcctagc

SEQ ID NO: 676 - DNA, 21 bp, Homo sapiens
aatctcccaccccgcctagct

SEQ ID NO: 677 - DNA, 21 bp, Homo sapiens
atctcccaccccgcctagctc

SEQ ID NO: 678 - DNA, 21 bp, Homo sapiens
tctcccaccccgcctagctca

SEQ ID NO: 679 - DNA, 21 bp, Homo sapiens
ctcccaccccccctagctcac

SEQ ID NO: 680 - DNA, 21 bp, Homo sapiens
tcccaccccgcctagctcacg

SEQ ID NO: 681 - DNA, 21 bp, Homo sapiens
cccaccccgcctagctcacgc

SEQ ID NO: 682 - DNA, 21 bp, Homo sapiens
ccaccccgcccagctcacgca

SEQ ID NO: 683 - DNA, 21 bp, Homo sapiens
caccccgcctcgctcacgcaa

SEQ ID NO: 684 - DNA, 21 bp, Homo sapiens
accccgcctacctcacgcaag

SEQ ID NO: 685 - DNA, 21 bp, Homo sapiens
tacctcaaaaaccaactaacc

SEQ ID NO: 686 - DNA, 21 bp, Homo sapiens
acctcaaaaaacaactaacca

SEQ ID NO: 687 - DNA, 21 bp, Homo sapiens
cctcaaaaacaaactaaccaa

SEQ ID NO: 688 - DNA, 21 bp, Homo sapiens
ctcaaaaaccaactaaccaac

SEQ ID NO: 689 - DNA, 21 bp, Homo sapiens
tcaaaaaccaactaaccaacc

SEQ ID NO: 690 - DNA, 21 bp, Homo sapiens
caaaaaccaaataaccaacca

SEQ ID NO: 691 - DNA, 21 bp, Homo sapiens
aaaaaccaacaaaccaaccaa

SEQ ID NO: 692 - DNA, 21 bp, Homo sapiens
aaaaccaactaaccaaccaat

SEQ ID NO: 693 - DNA, 21 bp, Homo sapiens
aaccaaccaaaaatctcccac

SEQ ID NO: 694 - DNA, 21 bp, Homo sapiens
accaaccaataatctcccacc

SEQ ID NO: 695 - DNA, 21 bp, Homo sapiens
ccaaccaataatctcccaccc

SEQ ID NO: 696 - DNA, 21 bp, Homo sapiens
caaccaataaactcccacccc

SEQ ID NO: 697 - DNA, 21 bp, Homo sapiens
aaccaataatatcccaccccg

SEQ ID NO: 698 - DNA, 20 bp, Homo sapiens
tacattgcccatgtaattaa

SEQ ID NO: 699 - DNA, 20 bp, Homo sapiens
atatagtttcgtcattcatc

SEQ ID NO: 700 - DNA, 20 bp, Homo sapiens
tacattgcccatgtaattaa

SEQ ID NO: 701 - DNA, 20 bp, Homo sapiens
atatagtttcgtcattcatc

SEQ ID NO: 702 - DNA, 20 bp, Homo sapiens
agatagtttagtcattcatc

SEQ ID NO: 703 - DNA, 20 bp, Homo sapiens
agatagtttcatcattcatc

SEQ ID NO: 704 - DNA, 20 bp, Homo sapiens
agatagtttcgacattcatc

SEQ ID NO: 705 - DNA, 20 bp, Homo sapiens
agatagtttcgtaattcatc

SEQ ID NO: 706 - DNA, 21 bp, Homo sapiens
accaataatcacccaccccgc

SEQ ID NO: 707 - DNA, 21 bp, Homo sapiens
ccaataatctaccaccccgcc

SEQ ID NO: 708 - DNA, 21 bp, Homo sapiens
caataatctcacaccccgcct

SEQ ID NO: 709 - DNA, 21 bp, Homo sapiens
aataatctccaaccccgccta

SEQ ID NO: 710 - DNA, 21 bp, Homo sapiens
ataatctcccaccccgcctag

SEQ ID NO: 711 - DNA, 21 bp, Homo sapiens
taatctcccaacccgcctagc

SEQ ID NO: 712 - DNA, 21 bp, Homo sapiens
aatctcccacaccgcctagct

SEQ ID NO: 713 - DNA, 21 bp, Homo sapiens
atctcccaccacgcctagctc

SEQ ID NO: 714 - DNA, 21 bp, Homo sapiens
tctcccacccagcctagctca

SEQ ID NO: 715 - DNA, 21 bp, Homo sapiens
ctcccaccccacctagctcac

SEQ ID NO: 716 - DNA, 21 bp, Homo sapiens
tcccaccccgactagctcacg

SEQ ID NO: 717 - DNA, 21 bp, Homo sapiens
cccaccccgcatagctcacgc

SEQ ID NO: 718 - DNA, 21 bp, Homo sapiens
ccaccccgccaagctcacgca

SEQ ID NO: 719 - DNA, 21 bp, Homo sapiens
caccccgcctagctcacgcaa

SEQ ID NO: 720 - DNA, 21 bp, Homo sapiens
accccgcctaactcacgcaag

SEQ ID NO: 721 - DNA, 21 bp, Homo sapiens
ccccgcctagttcacgcaagc

SEQ ID NO: 722 - DNA, 21 bp, Homo sapiens
cccgcctagctcacgcaagcc

SEQ ID NO: 723 - DNA, 21 bp, Homo sapiens
ccgcctagcttacgcaagccg

SEQ ID NO: 724 - DNA, 21 bp, Homo sapiens
cgcctagctctcgcaagccgc

SEQ ID NO: 725 - DNA, 21 bp, Homo sapiens
gcctagctcatgcaagccgcc

SEQ ID NO: 726 - DNA, 21 bp, Homo sapiens
cctagctcactcaagccgcca

SEQ ID NO: 727 - DNA, 21 bp, Homo sapiens
ctagctcacgtaagccgccaa

SEQ ID NO: 728 - DNA, 21 bp, Homo sapiens
tagctcacgctagccgccaac

SEQ ID NO: 729 - DNA, 21 bp, Homo sapiens
agctcacgcatgccgccaacg

SEQ ID NO: 730 - DNA, 21 bp, Homo sapiens
gctcacgcaatccgccaacgc

SEQ ID NO: 731 - DNA, 20 bp, Homo sapiens
atatagtttcgtcattcatc

SEQ ID NO: 732 - DNA, 20 bp, Homo sapiens
tacattgcccatgtaattaa

SEQ ID NO: 733 - DNA, 20 bp, Homo sapiens
atatagtttcgtcattcatc

SEQ ID NO: 734 - DNA, 20 bp, Homo sapiens
tacattgcccatgtaattaa

SEQ ID NO: 735 - DNA, 20 bp, Homo sapiens
agatagttttgtcattcatc

SEQ ID NO: 736 - DNA, 20 bp, Homo sapiens
agatagtttcttcattcatc

SEQ ID NO: 737 - DNA, 20 bp, Homo sapiens
agatagtttcgtcattcatc

SEQ ID NO: 738 - DNA, 20 bp, Homo sapiens
agatagtttcgttattcatc

SEQ ID NO: 739 - DNA, 21 bp, Homo sapiens
ctcacgcaagtcgccaacgcc

SEQ ID NO: 740 - DNA, 21 bp, Homo sapiens
tcacgcaagctgccaacgcct

SEQ ID NO: 741 - DNA, 21 bp, Homo sapiens
cacgcaagcctccaacgcctc

SEQ ID NO: 742 - DNA, 21 bp, Homo sapiens
acgcaagccgtcaacgcctct

SEQ ID NO: 743 - DNA, 21 bp, Homo sapiens
cgcaagccgctaacgcctctc

SEQ ID NO: 744 - DNA, 21 bp, Homo sapiens
gcaagccgcctacgcctctcc

SEQ ID NO: 745 - DNA, 21 bp, Homo sapiens
caagccgccatcgcctctccc

SEQ ID NO: 746 - DNA, 21 bp, Homo sapiens
aagccgccaatgcctctcccc

SEQ ID NO: 747 - DNA, 21 bp, Homo sapiens
agccgccaactcctctccccc

SEQ ID NO: 748 - DNA, 21 bp, Homo sapiens
gccgccaacgtctctccccct

SEQ ID NO: 749 - DNA, 21 bp, Homo sapiens
ccgccaacgcttctccccctc

SEQ ID NO: 750 - DNA, 21 bp, Homo sapiens
cgccaacgcctctccccctct

SEQ ID NO: 751 - DNA, 21 bp, Homo sapiens
gccaacgcctttccccctctc

SEQ ID NO: 752 - DNA, 21 bp, Homo sapiens
ccaacgcctctccccctctca

SEQ ID NO: 753 - DNA, 21 bp, Homo sapiens
caacgcctcttcccctctcat

SEQ ID NO: 754 - DNA, 21 bp, Homo sapiens
aacgcctctctccctctcatc

SEQ ID NO: 755 - DNA, 21 bp, Homo sapiens
acgcctctcctcctctcatcc

SEQ ID NO: 756 - DNA, 21 bp, Homo sapiens
cgcctctccctctctcatcca

SEQ ID NO: 757 - DNA, 21 bp, Homo sapiens
ccccgcctaggtcacgcaagc

SEQ ID NO: 758 - DNA, 21 bp, Homo sapiens
cccgcctagcgcacgcaagcc

SEQ ID NO: 759 - DNA, 21 bp, Homo sapiens
ccgcctagctgacgcaagccg

SEQ ID NO: 760 - DNA, 21 bp, Homo sapiens
cgcctagctcgcgcaagccgc

SEQ ID NO: 761 - DNA, 21 bp, Homo sapiens
gcctagctcaggcaagccgcc

SEQ ID NO: 762 - DNA, 21 bp, Homo sapiens
cctagctcacgcaagccgcca

SEQ ID NO: 763 - DNA, 21 bp, Homo sapiens
ctagctcacggaagccgccaa

SEQ ID NO: 764 - DNA, 21 bp, Homo sapiens
tagctcacgcgagccgccaac

SEQ ID NO: 765 - DNA, 21 bp, Homo sapiens
agctcacgcaggccgccaacg

SEQ ID NO: 766 - DNA, 21 bp, Homo sapiens
gctcacgcaagccgccaacgc

SEQ ID NO: 767 - DNA, 20 bp, Homo sapiens
tacattgcccatgtaattaa

SEQ ID NO: 768 - DNA, 20 bp, Homo sapiens
atatagtttcgtcattcatc

SEQ ID NO: 769 - DNA, 20 bp, Homo sapiens
tacattgcccatgtaattaa

SEQ ID NO: 770 - DNA, 20 bp, Homo sapiens
atatagtttcgtcattcatc

SEQ ID NO: 771 - DNA, 20 bp, Homo sapiens
agatagtttggtcattcatc

SEQ ID NO: 772 - DNA, 20 bp, Homo sapiens
agatagtttcgtcattcatc

SEQ ID NO: 773 - DNA, 20 bp, Homo sapiens
agatagtttcggcattcatc

SEQ ID NO: 774 - DNA, 20 bp, Homo sapiens
agatagtttcgtgattcatc

SEQ ID NO: 775 - DNA, 21 bp, Homo sapiens
ctcacgcaaggcgccaacgcc

SEQ ID NO: 776 - DNA, 21 bp, Homo sapiens
tcacgcaagcggccaacgcct

SEQ ID NO: 777 - DNA, 21 bp, Homo sapiens
cacgcaagccgccaacgcctc

SEQ ID NO: 778 - DNA, 21 bp, Homo sapiens
acgcaagccggcaacgcctct

SEQ ID NO: 779 - DNA, 21 bp, Homo sapiens
cgcaagccgcgaacgcctctc

SEQ ID NO: 780 - DNA, 21 bp, Homo sapiens
gcaagccgccgacgcctctcc

SEQ ID NO: 781 - DNA, 21 bp, Homo sapiens
caagccgccagcgcctctccc

SEQ ID NO: 782 - DNA, 21 bp, Homo sapiens
aagccgccaaggcctctcccc

SEQ ID NO: 783 - DNA, 21 bp, Homo sapiens
agccgccaacgcctctccccc

SEQ ID NO: 784 - DNA, 21 bp, Homo sapiens
gccgccaacggctctccccct

SEQ ID NO: 785 - DNA, 21 bp, Homo sapiens
ccgccaacgcgtctccccctc

SEQ ID NO: 786 - DNA, 21 bp, Homo sapiens
cgccaacgccgctccccctct

SEQ ID NO: 787 - DNA, 21 bp, Homo sapiens
gccaacgcctgtccccctctc

SEQ ID NO: 788 - DNA, 21 bp, Homo sapiens
ccaacgcctcgccccctctca

SEQ ID NO: 789 - DNA, 21 bp, Homo sapiens
caacgcctctgcccctctcat

SEQ ID NO: 790 - DNA, 21 bp, Homo sapiens
aacgcctctcgccctctcatc

SEQ ID NO: 791 - DNA, 21 bp, Homo sapiens
acgcctctccgcctctcatcc

SEQ ID NO: 792 - DNA, 21 bp, Homo sapiens
cgcctctcccgctctcatcca

SEQ ID NO: 793 - DNA, 21 bp, Homo sapiens
ccccgcctagctcacgcaagc

SEQ ID NO: 794 - DNA, 21 bp, Homo sapiens
cccgcctagcccacgcaagcc

SEQ ID NO: 795 - DNA, 21 bp, Homo sapiens
ccgcctagctcacgcaagccg

SEQ ID NO: 796 - DNA, 21 bp, Homo sapiens
cgcctagctcccgcaagccgc

SEQ ID NO: 797 - DNA, 21 bp, Homo sapiens
gcctagctcacgcaagccgcc

SEQ ID NO: 798 - DNA, 21 bp, Homo sapiens
cctagctcacccaagccgcca

SEQ ID NO: 799 - DNA, 21 bp, Homo sapiens
ctagctcacgcaagccgccaa

SEQ ID NO: 800 - DNA, 21 bp, Homo sapiens
tagctcacgccagccgccaac

SEQ ID NO: 801 - DNA, 21 bp, Homo sapiens
agctcacgcacgccgccaacg

SEQ ID NO: 802 - DNA, 21 bp, Homo sapiens
gctcacgcaacccgccaacgc

SEQ ID NO: 803 - DNA, 20 bp, Homo sapiens
atatagtttcgtcattcatc

SEQ ID NO: 804 - DNA, 20 bp, Homo sapiens
tacattgcccatgtaattaa

SEQ ID NO: 805 - DNA, 20 bp, Homo sapiens
atatagtttcgtcattcatc

SEQ ID NO: 806 - DNA, 20 bp, Homo sapiens
tacattgcccatgtaattaa

SEQ ID NO: 807 - DNA, 20 bp, Homo sapiens
agatagtttcgtcattcatc

SEQ ID NO: 808 - DNA, 20 bp, Homo sapiens
agatagtttcctcattcatc

SEQ ID NO: 809 - DNA, 20 bp, Homo sapiens
agatagtttcgccattcatc

SEQ ID NO: 810 - DNA, 20 bp, Homo sapiens
agatagtttcgtcattcatc

SEQ ID NO: 811 - DNA, 21 bp, Homo sapiens
ctcacgcaagccgccaacgcc

SEQ ID NO: 812 - DNA, 21 bp, Homo sapiens
tcacgcaagccgccaacgcct

SEQ ID NO: 813 - DNA, 21 bp, Homo sapiens
cacgcaagcccccaacgcctc

SEQ ID NO: 814 - DNA, 21 bp, Homo sapiens
acgcaagccgccaacgcctct

SEQ ID NO: 815 - DNA, 21 bp, Homo sapiens
cgcaagccgccaacgcctctc

SEQ ID NO: 816 - DNA, 21 bp, Homo sapiens
gcaagccgcccacgcctctcc

SEQ ID NO: 817 - DNA, 21 bp, Homo sapiens
caagccgccaccgcctctccc

SEQ ID NO: 818 - DNA, 21 bp, Homo sapiens
aagccgccaacgcctctcccc

SEQ ID NO: 819 - DNA, 21 bp, Homo sapiens
agccgccaacccctctccccc

SEQ ID NO: 820 - DNA, 21 bp, Homo sapiens
gccgccaacgcctctccccct

SEQ ID NO: 821 - DNA, 21 bp, Homo sapiens
ccgccaacgcctctccccctc

SEQ ID NO: 822 - DNA, 21 bp, Homo sapiens
cgccaacgcccctccccctct

SEQ ID NO: 823 - DNA, 21 bp, Homo sapiens
gccaacgcctctccccctctc

SEQ ID NO: 824 - DNA, 21 bp, Homo sapiens
ccaacgcctccccccctctca

SEQ ID NO: 825 - DNA, 21 bp, Homo sapiens
caacgcctctccccctctcat

SEQ ID NO: 826 - DNA, 21 bp, Homo sapiens
aacgcctctccccctctcatc

SEQ ID NO: 827 - DNA, 21 bp, Homo sapiens
acgcctctccccctctcatcc

SEQ ID NO: 828 - DNA, 21 bp, Homo sapiens
cgcctctccccctctcatcca

SEQ ID NO: 829 - DNA, 21 bp, Homo sapiens
ccccgcctagatcacgcaagc

SEQ ID NO: 830 - DNA, 21 bp, Homo sapiens
cccgcctagcacacgcaagcc

SEQ ID NO: 831 - DNA, 21 bp, Homo sapiens
ccgcctagctaacgcaagccg

SEQ ID NO: 832 - DNA, 21 bp, Homo sapiens
cgcctagctcacgcaagccgc

SEQ ID NO: 833 - DNA, 21 bp, Homo sapiens
gcctagctcaagcaagccgcc

SEQ ID NO: 834 - DNA, 21 bp, Homo sapiens
cctagctcacacaagccgcca

SEQ ID NO: 835 - DNA, 21 bp, Homo sapiens
ctagctcacgaaagccgccaa

SEQ ID NO: 836 - DNA, 21 bp, Homo sapiens
tagctcacgcaagccgccaac

SEQ ID NO: 837 - DNA, 21 bp, Homo sapiens
agctcacgcaagccgccaacg

SEQ ID NO: 838 - DNA, 21 bp, Homo sapiens
gctcacgcaaaccgccaacgc

SEQ ID NO: 839 - DNA, 20 bp, Homo sapiens
tacattgcccatgtaattaa

SEQ ID NO: 840 - DNA, 20 bp, Homo sapiens
atatagtttcgtcattcatc

SEQ ID NO: 841 - DNA, 20 bp, Homo sapiens
tacattgcccatgtaattaa

SEQ ID NO: 842 - DNA, 20 bp, Homo sapiens
atatagtttcgtcattcatc

SEQ ID NO: 843 - DNA, 20 bp, Homo sapiens
agatagtttagtcattcatc

SEQ ID NO: 844 - DNA, 20 bp, Homo sapiens
agatagtttcatcattcatc

SEQ ID NO: 845 - DNA, 20 bp, Homo sapiens
agatagtttcgacattcatc

SEQ ID NO: 846 - DNA, 20 bp, Homo sapiens
agatagtttcgtaattcatc

SEQ ID NO: 847 - DNA, 21 bp, Homo sapiens
ctcacgcaagacgccaacgcc

SEQ ID NO: 848 - DNA, 21 bp, Homo sapiens
tcacgcaagcagccaacgcct

SEQ ID NO: 849 - DNA, 21 bp, Homo sapiens
cacgcaagccaccaacgcctc

SEQ ID NO: 850 - DNA, 21 bp, Homo sapiens
acgcaagccgacaacgcctct

SEQ ID NO: 851 - DNA, 21 bp, Homo sapiens
cgcaagccgcaaacgcctctc

SEQ ID NO: 852 - DNA, 21 bp, Homo sapiens
gcaagccgccaacgcctctcc

SEQ ID NO: 853 - DNA, 21 bp, Homo sapiens
caagccgccaacgcctctccc

SEQ ID NO: 854 - DNA, 21 bp, Homo sapiens
aagccgccaaagcctctcccc

SEQ ID NO: 855 - DNA, 21 bp, Homo sapiens
agccgccaacacctctccccc

SEQ ID NO: 856 - DNA, 21 bp, Homo sapiens
gccgccaacgactctccccct

SEQ ID NO: 857 - DNA, 21 bp, Homo sapiens
ccgccaacgcatctccccctc

SEQ ID NO: 858 - DNA, 21 bp, Homo sapiens
cgccaacgccactccccctct

SEQ ID NO: 859 - DNA, 21 bp, Homo sapiens
gccaacgcctatccccctctc

SEQ ID NO: 860 - DNA, 21 bp, Homo sapiens
ccaacgcctcaccccctctca

SEQ ID NO: 861 - DNA, 21 bp, Homo sapiens
caacgcctctacccctctcat

SEQ ID NO: 862 - DNA, 21 bp, Homo sapiens
aacgcctctcaccctctcatc

SEQ ID NO: 863 - DNA, 21 bp, Homo sapiens
acgcctctccacctctcatcc

SEQ ID NO: 864 - DNA, 21 bp, Homo sapiens
cgcctctcccactctcatcca

SEQ ID NO: 865 - DNA, 21 bp, Homo sapiens
gcctctccccttctcatccat

SEQ ID NO: 866 - DNA, 21 bp, Homo sapiens
cctctccccctctcatccatc

SEQ ID NO: 867 - DNA, 21 bp, Homo sapiens
ctctccccctttcatccatcg

SEQ ID NO: 868 - DNA, 21 bp, Homo sapiens
tctccccctctcatccatcgc

SEQ ID NO: 869 - DNA, 21 bp, Homo sapiens
ctccccctcttatccatcgcc

SEQ ID NO: 870 - DNA, 21 bp, Homo sapiens
tccccctctcttccatcgccc

SEQ ID NO: 871 - DNA, 21 bp, Homo sapiens
ccccctctcatccatcgcccg

SEQ ID NO: 872 - DNA, 20 bp, Homo sapiens
atatagtttcgtcattcatc

SEQ ID NO: 873 - DNA, 20 bp, Homo sapiens
tacattgcccatgtaattaa

SEQ ID NO: 874 - DNA, 20 bp, Homo sapiens
atatagtttcgtcattcatc

SEQ ID NO: 875 - DNA, 20 bp, Homo sapiens
tacattgcccatgtaattaa

SEQ ID NO: 876 - DNA, 20 bp, Homo sapiens
agatagttttgtcattcatc

SEQ ID NO: 877 - DNA, 20 bp, Homo sapiens
agatagtttcttcattcatc

SEQ ID NO: 878 - DNA, 20 bp, Homo sapiens
agatagtttcgtcattcatc

SEQ ID NO: 879 - DNA, 20 bp, Homo sapiens
agatagtttcgttattcatc

SEQ ID NO: 880 - DNA, 21 bp, Homo sapiens
cccctctcattcatcgcccgc

SEQ ID NO: 881 - DNA, 21 bp, Homo sapiens
ccctctcatctatcgcccgcc

SEQ ID NO: 882 - DNA, 21 bp, Homo sapiens
cctctcatccttcgcccgccg

SEQ ID NO: 883 - DNA, 21 bp, Homo sapiens
ctctcatccatcgcccgccgc

SEQ ID NO: 884 - DNA, 21 bp, Homo sapiens
tctcatccattgcccgccgcc

SEQ ID NO: 885 - DNA, 21 bp, Homo sapiens
ctcatccatctcccgccgccc

SEQ ID NO: 886 - DNA, 21 bp, Homo sapiens
tcatccatcgtccgccgcccc

SEQ ID NO: 887 - DNA, 21 bp, Homo sapiens
catccatcgctcgccgcccct

SEQ ID NO: 888 - DNA, 21 bp, Homo sapiens
atccatcgcctgccgcccctc

SEQ ID NO: 889 - DNA, 21 bp, Homo sapiens
tccatcgccctccgcccctca

SEQ ID NO: 890 - DNA, 21 bp, Homo sapiens
ccatcgcccgtcgcccctcat

SEQ ID NO: 891 - DNA, 21 bp, Homo sapiens
catcgcccgctgcccctcatc

SEQ ID NO: 892 - DNA, 21 bp, Homo sapiens
atcgcccgcctcccctcatca

SEQ ID NO: 893 - DNA, 21 bp, Homo sapiens
tcgcccgccgtccctcatcat

SEQ ID NO: 894 - DNA, 21 bp, Homo sapiens
cgcccgccgctcctcatcata

SEQ ID NO: 895 - DNA, 21 bp, Homo sapiens
gcccgccgcctctcatcatac

SEQ ID NO: 896 - DNA, 21 bp, Homo sapiens
cccgccgcccttcatcatacc

SEQ ID NO: 897 - DNA, 21 bp, Homo sapiens
ccgccgcccctcatcatacct

SEQ ID NO: 898 - DNA, 21 bp, Homo sapiens
cgccgccccttatcatacctc

SEQ ID NO: 899 - DNA, 21 bp, Homo sapiens
gccgcccctcttcatacctca

SEQ ID NO: 900 - DNA, 21 bp, Homo sapiens
ccgcccctcatcatacctcag

SEQ ID NO: 901 - DNA, 21 bp, Homo sapiens
gcctctccccgtctcatccat

SEQ ID NO: 902 - DNA, 21 bp, Homo sapiens
cctctcccccgctcatccatc

SEQ ID NO: 903 - DNA, 21 bp, Homo sapiens
ctctccccctgtcatccatcg

SEQ ID NO: 904 - DNA, 21 bp, Homo sapiens
tctccccctcgcatccatcgc

SEQ ID NO: 905 - DNA, 21 bp, Homo sapiens
ctccccctctgatccatcgcc

SEQ ID NO: 906 - DNA, 21 bp, Homo sapiens
tccccctctcgtccatcgccc

SEQ ID NO: 907 - DNA, 21 bp, Homo sapiens
ccccctctcagccatcgcccg

SEQ ID NO: 908 - DNA, 20 bp, Homo sapiens
tacattgcccatgtaattaa

SEQ ID NO: 909 - DNA, 20 bp, Homo sapiens
atatagtttcgtcattcatc

SEQ ID NO: 910 - DNA, 20 bp, Homo sapiens
tacattgcccatgtaattaa

SEQ ID NO: 911 - DNA, 20 bp, Homo sapiens
atatagtttcgtcattcatc

SEQ ID NO: 912 - DNA, 20 bp, Homo sapiens
agatagtttggtcattcatc

SEQ ID NO: 913 - DNA, 20 bp, Homo sapiens
agatagtttcgtcattcatc

SEQ ID NO: 914 - DNA, 20 bp, Homo sapiens
agatagtttcggcattcatc

SEQ ID NO: 915 - DNA, 20 bp, Homo sapiens
agatagtttcgtgattcatc

SEQ ID NO: 916 - DNA, 21 bp, Homo sapiens
cccctctcatgcatcgcccgc

SEQ ID NO: 917 - DNA, 21 bp, Homo sapiens
ccctctcatcgatcgcccgcc

SEQ ID NO: 918 - DNA, 21 bp, Homo sapiens
cctctcatccgtcgcccgccg

SEQ ID NO: 919 - DNA, 21 bp, Homo sapiens
ctctcatccagcgcccgccgc

SEQ ID NO: 920 - DNA, 21 bp, Homo sapiens
tctcatccatggcccgccgcc

SEQ ID NO: 921 - DNA, 21 bp, Homo sapiens
ctcatccatcgcccgccgccc

SEQ ID NO: 922 - DNA, 21 bp, Homo sapiens
tcatccatcggccgccgcccc

SEQ ID NO: 923 - DNA, 21 bp, Homo sapiens
catccatcgcgcgccgcccct

SEQ ID NO: 924 - DNA, 21 bp, Homo sapiens
atccatcgccggccgcccctc

SEQ ID NO: 925 - DNA, 21 bp, Homo sapiens
tccatcgcccgccgcccctca

SEQ ID NO: 926 - DNA, 21 bp, Homo sapiens
ccatcgcccggcgcccctcat

SEQ ID NO: 927 - DNA, 21 bp, Homo sapiens
catcgcccgcggcccctcatc

SEQ ID NO: 928 - DNA, 21 bp, Homo sapiens
atcgcccgccgcccctcatca

SEQ ID NO: 929 - DNA, 21 bp, Homo sapiens
tcgcccgccggccctcatcat

SEQ ID NO: 930 - DNA, 21 bp, Homo sapiens
cgcccgccgcgcctcatcata

SEQ ID NO: 931 - DNA, 21 bp, Homo sapiens
gcccgccgccgctcatcatac

SEQ ID NO: 932 - DNA, 21 bp, Homo sapiens
cccgccgcccgtcatcatacc

SEQ ID NO: 933 - DNA, 21 bp, Homo sapiens
ccgccgccccgcatcatacct

SEQ ID NO: 934 - DNA, 21 bp, Homo sapiens
cgccgcccctgatcatacctc

SEQ ID NO: 935 - DNA, 21 bp, Homo sapiens
gccgcccctcgtcatacctca

SEQ ID NO: 936 - DNA, 21 bp, Homo sapiens
ccgcccctcagcatacctcag

SEQ ID NO: 937 - DNA, 21 bp, Homo sapiens
gcctctccccctctcatccat

SEQ ID NO: 938 - DNA, 21 bp, Homo sapiens
cctctccccccctcatccatc

SEQ ID NO: 939 - DNA, 21 bp, Homo sapiens
ctctccccctctcatccatcg

SEQ ID NO: 940 - DNA, 21 bp, Homo sapiens
tctccccctcccatccatcgc

SEQ ID NO: 941 - DNA, 21 bp, Homo sapiens
ctccccctctcatccatcgcc

SEQ ID NO: 942 - DNA, 21 bp, Homo sapiens
tccccctctcctccatcgccc

SEQ ID NO: 943 - DNA, 21 bp, Homo sapiens
ccccctctcacccatcgcccg

SEQ ID NO: 944 - DNA, 20 bp, Homo sapiens
atatagtttcgtcattcatc

SEQ ID NO: 945 - DNA, 20 bp, Homo sapiens
tacattgcccatgtaattaa

SEQ ID NO: 946 - DNA, 20 bp, Homo sapiens
atatagtttcgtcattcatc

SEQ ID NO: 947 - DNA, 20 bp, Homo sapiens
tacattgcccatgtaattaa

SEQ ID NO: 948 - DNA, 20 bp, Homo sapiens
agatagtttcgtcattcatc

SEQ ID NO: 949 - DNA, 20 bp, Homo sapiens
agatagtttcctcattcatc

SEQ ID NO: 950 - DNA, 20 bp, Homo sapiens
agatagtttcgccattcatc

SEQ ID NO: 951 - DNA, 20 bp, Homo sapiens
agatagtttcgtcattcatc

SEQ ID NO: 952 - DNA, 21 bp, Homo sapiens
cccctctcatccatcgcccgc

SEQ ID NO: 953 - DNA, 21 bp, Homo sapiens
ccctctcatccatcgcccgcc

SEQ ID NO: 954 - DNA, 21 bp, Homo sapiens
cctctcatccctcgcccgccg

SEQ ID NO: 955 - DNA, 21 bp, Homo sapiens
ctctcatccaccgcccgccgc

SEQ ID NO: 956 - DNA, 21 bp, Homo sapiens
tctcatccatcgcccgccgcc

SEQ ID NO: 957 - DNA, 21 bp, Homo sapiens
ctcatccatcccccgccgccc

SEQ ID NO: 958 - DNA, 21 bp, Homo sapiens
tcatccatcgcccgccgcccc

SEQ ID NO: 959 - DNA, 21 bp, Homo sapiens
catccatcgcccgccgcccct

SEQ ID NO: 960 - DNA, 21 bp, Homo sapiens
atccatcgcccgccgcccctc

SEQ ID NO: 961 - DNA, 21 bp, Homo sapiens
tccatcgccccccgcccctca

SEQ ID NO: 962 - DNA, 21 bp, Homo sapiens
ccatcgcccgccgcccctcat

SEQ ID NO: 963 - DNA, 21 bp, Homo sapiens
catcgcccgccgcccctcatc

SEQ ID NO: 964 - DNA, 21 bp, Homo sapiens
atcgcccgccccccctcatca

SEQ ID NO: 965 - DNA, 21 bp, Homo sapiens
tcgcccgccgcccctcatcat

SEQ ID NO: 966 - DNA, 21 bp, Homo sapiens
cgcccgccgcccctcatcata

SEQ ID NO: 967 - DNA, 21 bp, Homo sapiens
gcccgccgcccctcatcatac

SEQ ID NO: 968 - DNA, 21 bp, Homo sapiens
cccgccgcccctcatcatacc

SEQ ID NO: 969 - DNA, 21 bp, Homo sapiens
ccgccgccccccatcatacct

SEQ ID NO: 970 - DNA, 21 bp, Homo sapiens
cgccgcccctcatcatacctc

SEQ ID NO: 971 - DNA, 21 bp, Homo sapiens
gccgcccctcctcatacctca

SEQ ID NO: 972 - DNA, 21 bp, Homo sapiens
ccgcccctcaccatacctcag

SEQ ID NO: 973 - DNA, 21 bp, Homo sapiens
gcctctccccatctcatccat

SEQ ID NO: 974 - DNA, 21 bp, Homo sapiens
cctctcccccactcatccatc

SEQ ID NO: 975 - DNA, 21 bp, Homo sapiens
ctctccccctatcatccatcg

SEQ ID NO: 976 - DNA, 21 bp, Homo sapiens
tctccccctcacatccatcgc

SEQ ID NO: 977 - DNA, 21 bp, Homo sapiens
ctccccctctaatccatcgcc

SEQ ID NO: 978 - DNA, 21 bp, Homo sapiens
tccccctctcatccatcgccc

SEQ ID NO: 979 - DNA, 21 bp, Homo sapiens
ccccctctcaaccatcgcccg

SEQ ID NO: 980 - DNA, 20 bp, Homo sapiens
tacattgcccatgtaattaa

SEQ ID NO: 981 - DNA, 20 bp, Homo sapiens
atatagtttcgtcattcatc

SEQ ID NO: 982 - DNA, 20 bp, Homo sapiens
tacattgcccatgtaattaa

SEQ ID NO: 983 - DNA, 20 bp, Homo sapiens
atatagtttcgtcattcatc

SEQ ID NO: 984 - DNA, 20 bp, Homo sapiens
agatagtttagtcattcatc

SEQ ID NO: 985 - DNA, 20 bp, Homo sapiens
agatagtttcatcattcatc

SEQ ID NO: 986 - DNA, 20 bp, Homo sapiens
agatagtttcgacattcatc

SEQ ID NO: 987 - DNA, 20 bp, Homo sapiens
agatagtttcgtaattcatc

SEQ ID NO: 988 - DNA, 21 bp, Homo sapiens
cccctctcatacatcgcccgc

SEQ ID NO: 989 - DNA, 21 bp, Homo sapiens
ccctctcatcaatcgcccgcc

SEQ ID NO: 990 - DNA, 21 bp, Homo sapiens
cctctcatccatcgcccgccg

SEQ ID NO: 991 - DNA, 21 bp, Homo sapiens
ctctcatccaacgcccgccgc

SEQ ID NO: 992 - DNA, 21 bp, Homo sapiens
tctcatccatagcccgccgcc

SEQ ID NO: 993 - DNA, 21 bp, Homo sapiens
ctcatccatcacccgccgccc

SEQ ID NO: 994 - DNA, 21 bp, Homo sapiens
tcatccatcgaccgccgcccc

SEQ ID NO: 995 - DNA, 21 bp, Homo sapiens
catccatcgcacgccgcccct

SEQ ID NO: 996 - DNA, 21 bp, Homo sapiens
atccatcgccagccgcccctc

SEQ ID NO: 997 - DNA, 21 bp, Homo sapiens
tccatcgcccaccgcccctca

SEQ ID NO: 998 - DNA, 21 bp, Homo sapiens
ccatcgcccgacgcccctcat

SEQ ID NO: 999 - DNA, 21 bp, Homo sapiens
catcgcccgcagcccctcatc

SEQ ID NO: 1000 - DNA, 21 bp, Homo sapiens
atcgcccgccacccctcatca

SEQ ID NO: 1001 - DNA, 21 bp, Homo sapiens
tcgcccgccgaccctcatcat

SEQ ID NO: 1002 - DNA, 21 bp, Homo sapiens
cgcccgccgcacctcatcata

SEQ ID NO: 1003 - DNA, 21 bp, Homo sapiens
gcccgccgccactcatcatac

SEQ ID NO: 1004 - DNA, 21 bp, Homo sapiens
cccgccgcccatcatcatacc

SEQ ID NO: 1005 - DNA, 21 bp, Homo sapiens
ccgccgccccacatcatacct

SEQ ID NO: 1006 - DNA, 21 bp, Homo sapiens
cgccgcccctaatcatacctc

SEQ ID NO: 1007 - DNA, 21 bp, Homo sapiens
gccgcccctcatcatacctca

SEQ ID NO: 1008 - DNA, 21 bp, Homo sapiens
ccgcccctcaacatacctcag

SEQ ID NO: 1009 - DNA, 21 bp, Homo sapiens
cgcccctcattatacctcagc

SEQ ID NO: 1010 - DNA, 21 bp, Homo sapiens
gcccctcatcttacctcagcc

SEQ ID NO: 1011 - DNA, 21 bp, Homo sapiens
cccctcatcatacctcagccg

SEQ ID NO: 1012 - DNA, 21 bp, Homo sapiens
ccctcatcattcctcagccgc

SEQ ID NO: 1013 - DNA, 20 bp, Homo sapiens
atatagtttcgtcattcatc

SEQ ID NO: 1014 - DNA, 20 bp, Homo sapiens
tacattgcccatgtaattaa

SEQ ID NO: 1015 - DNA, 20 bp, Homo sapiens
atatagtttcgtcattcatc

SEQ ID NO: 1016 - DNA, 20 bp, Homo sapiens
tacattgcccatgtaattaa

SEQ ID NO: 1017 - DNA, 20 bp, Homo sapiens
agatagttttgtcattcatc

SEQ ID NO: 1018 - DNA, 20 bp, Homo sapiens
agatagtttcttcattcatc

SEQ ID NO: 1019 - DNA, 20 bp, Homo sapiens
agatagtttcgtcattcatc

SEQ ID NO: 1020 - DNA, 20 bp, Homo sapiens
agatagtttcgttattcatc

SEQ ID NO: 1021 - DNA, 21 bp, Homo sapiens
cctcatcatatctcagccgcc

SEQ ID NO: 1022 - DNA, 21 bp, Homo sapiens
ctcatcatacttcagccgccg

SEQ ID NO: 1023 - DNA, 21 bp, Homo sapiens
tcatcatacctcagccgccgc

SEQ ID NO: 1024 - DNA, 21 bp, Homo sapiens
catcataccttagccgccgcc

SEQ ID NO: 1025 - DNA, 21 bp, Homo sapiens
atcatacctctgccgccgccc

SEQ ID NO: 1026 - DNA, 21 bp, Homo sapiens
tcatacctcatccgccgcccc

SEQ ID NO: 1027 - DNA, 21 bp, Homo sapiens
catacctcagtcgccgcccct

SEQ ID NO: 1028 - DNA, 21 bp, Homo sapiens
atacctcagctgccgcccctc

SEQ ID NO: 1029 - DNA, 21 bp, Homo sapiens
tacctcagcctccgcccctca

SEQ ID NO: 1030 - DNA, 21 bp, Homo sapiens
acctcagccgtcgcccctcat

SEQ ID NO: 1031 - DNA, 21 bp, Homo sapiens
cctcagccgctgcccctcatc

SEQ ID NO: 1032 - DNA, 21 bp, Homo sapiens
ctcagccgcctcccctcatca

SEQ ID NO: 1033 - DNA, 21 bp, Homo sapiens
tcagccgccgtccctcatcat

SEQ ID NO: 1034 - DNA, 21 bp, Homo sapiens
cagccgccgctcctcatcata

SEQ ID NO: 1035 - DNA, 21 bp, Homo sapiens
agccgccgcctctcatcatac

SEQ ID NO: 1036 - DNA, 21 bp, Homo sapiens
gccgccgcccttcatcatacc

SEQ ID NO: 1037 - DNA, 21 bp, Homo sapiens
ccgccgcccctcatcatacct

SEQ ID NO: 1038 - DNA, 21 bp, Homo sapiens
cgccgccccttatcatacctc

SEQ ID NO: 1039 - DNA, 21 bp, Homo sapiens
gccgcccctcttcatacctca

SEQ ID NO: 1040 - DNA, 21 bp, Homo sapiens
ccgcccctcatcatacctcaa

SEQ ID NO: 1041 - DNA, 21 bp, Homo sapiens
cgcccctcattatacctcaaa

SEQ ID NO: 1042 - DNA, 21 bp, Homo sapiens
gcccctcatcttacctcaaaa

SEQ ID NO: 1043 - DNA, 21 bp, Homo sapiens
cccctcatcatacctcaaaag

SEQ ID NO: 1044 - DNA, 21 bp, Homo sapiens
ccctcatcattcctcaaaagc

SEQ ID NO: 1045 - DNA, 21 bp, Homo sapiens
cgcccctcatgatacctcagc

SEQ ID NO: 1046 - DNA, 21 bp, Homo sapiens
gcccctcatcgtacctcagcc

SEQ ID NO: 1047 - DNA, 21 bp, Homo sapiens
cccctcatcagacctcagccg

SEQ ID NO: 1048 - DNA, 21 bp, Homo sapiens
ccctcatcatgcctcagccgc

SEQ ID NO: 1049 - DNA, 20 bp, Homo sapiens
tacattgcccatgtaattaa

SEQ ID NO: 1050 - DNA, 20 bp, Homo sapiens
atatagtttcgtcattcatc

SEQ ID NO: 1051 - DNA, 20 bp, Homo sapiens
tacattgcccatgtaattaa

SEQ ID NO: 1052 - DNA, 20 bp, Homo sapiens
atatagtttcgtcattcatc

SEQ ID NO: 1053 - DNA, 20 bp, Homo sapiens
agatagtttggtcattcatc

SEQ ID NO: 1054 - DNA, 20 bp, Homo sapiens
agatagtttcgtcattcatc

SEQ ID NO: 1055 - DNA, 20 bp, Homo sapiens
agatagtttcggcattcatc

SEQ ID NO: 1056 - DNA, 20 bp, Homo sapiens
agatagtttcgtgattcatc

SEQ ID NO: 1057 - DNA, 21 bp, Homo sapiens
cctcatcatagctcagccgcc

SEQ ID NO: 1058 - DNA, 21 bp, Homo sapiens
ctcatcatacgtcagccgccg

SEQ ID NO: 1059 - DNA, 21 bp, Homo sapiens
tcatcataccgcagccgccgc

SEQ ID NO: 1060 - DNA, 21 bp, Homo sapiens
catcatacctgagccgccgcc

SEQ ID NO: 1061 - DNA, 21 bp, Homo sapiens
atcatacctcggccgccgccc

SEQ ID NO: 1062 - DNA, 21 bp, Homo sapiens
tcatacctcagccgccgcccc

SEQ ID NO: 1063 - DNA, 21 bp, Homo sapiens
catacctcaggcgccgcccct

SEQ ID NO: 1064 - DNA, 21 bp, Homo sapiens
atacctcagcggccgcccctc

SEQ ID NO: 1065 - DNA, 21 bp, Homo sapiens
tacctcagccgccgcccctca

SEQ ID NO: 1066 - DNA, 21 bp, Homo sapiens
acctcagccggcgcccctcat

SEQ ID NO: 1067 - DNA, 21 bp, Homo sapiens
cctcagccgcggcccctcatc

SEQ ID NO: 1068 - DNA, 21 bp, Homo sapiens
ctcagccgccgcccctcatca

SEQ ID NO: 1069 - DNA, 21 bp, Homo sapiens
tcagccgccggccctcatcat

SEQ ID NO: 1070 - DNA, 21 bp, Homo sapiens
cagccgccgcgcctcatcata

SEQ ID NO: 1071 - DNA, 21 bp, Homo sapiens
agccgccgccgctcatcatac

SEQ ID NO: 1072 - DNA, 21 bp, Homo sapiens
gccgccgcccgtcatcatacc

SEQ ID NO: 1073 - DNA, 21 bp, Homo sapiens
ccgccgccccgcatcatacct

SEQ ID NO: 1074 - DNA, 21 bp, Homo sapiens
cgccgcccctgatcatacctc

SEQ ID NO: 1075 - DNA, 21 bp, Homo sapiens
gccgcccctcgtcatacctca

SEQ ID NO: 1076 - DNA, 21 bp, Homo sapiens
ccgcccctcagcatacctcaa

SEQ ID NO: 1077 - DNA, 21 bp, Homo sapiens
cgcccctcatgatacctcaaa

SEQ ID NO: 1078 - DNA, 21 bp, Homo sapiens
gcccctcatcgtacctcaaaa

SEQ ID NO: 1079 - DNA, 21 bp, Homo sapiens
cccctcatcagacctcaaaag

SEQ ID NO: 1080 - DNA, 21 bp, Homo sapiens
ccctcatcatgcctcaaaagc

SEQ ID NO: 1081 - DNA, 21 bp, Homo sapiens
cgcccctcatcatacctcagc

SEQ ID NO: 1082 - DNA, 21 bp, Homo sapiens
gcccctcatcctacctcagcc

SEQ ID NO: 1083 - DNA, 21 bp, Homo sapiens
cccctcatcacacctcagccg

SEQ ID NO: 1084 - DNA, 21 bp, Homo sapiens
ccctcatcatccctcagccgc

SEQ ID NO: 1085 - DNA, 20 bp, Homo sapiens
atatagtttcgtcattcatc

SEQ ID NO: 1086 - DNA, 20 bp, Homo sapiens
tacattgcccatgtaattaa

SEQ ID NO: 1087 - DNA, 20 bp, Homo sapiens
atatagtttcgtcattcatc

SEQ ID NO: 1088 - DNA, 20 bp, Homo sapiens
tacattgcccatgtaattaa

SEQ ID NO: 1089 - DNA, 20 bp, Homo sapiens
agatagtttcgtcattcatc

SEQ ID NO: 1090 - DNA, 20 bp, Homo sapiens
agatagtttcctcattcatc

SEQ ID NO: 1091 - DNA, 20 bp, Homo sapiens
agatagtttcgccattcatc

SEQ ID NO: 1092 - DNA, 20 bp, Homo sapiens
agatagtttcgtcattcatc

SEQ ID NO: 1093 - DNA, 21 bp, Homo sapiens
cctcatcatacctcagccgcc

SEQ ID NO: 1094 - DNA, 21 bp, Homo sapiens
ctcatcatacctcagccgccg

SEQ ID NO: 1095 - DNA, 21 bp, Homo sapiens
tcatcataccccagccgccgc

SEQ ID NO: 1096 - DNA, 21 bp, Homo sapiens
catcatacctcagccgccgcc

SEQ ID NO: 1097 - DNA, 21 bp, Homo sapiens
atcatacctccgccgccgccc

SEQ ID NO: 1098 - DNA, 21 bp, Homo sapiens
tcatacctcacccgccgcccc

SEQ ID NO: 1099 - DNA, 21 bp, Homo sapiens
catacctcagccgccgcccct

SEQ ID NO: 1100 - DNA, 21 bp, Homo sapiens
atacctcagccgccgcccctc

SEQ ID NO: 1101 - DNA, 21 bp, Homo sapiens
tacctcagcccccgcccctca

SEQ ID NO: 1102 - DNA, 21 bp, Homo sapiens
acctcagccgccgcccctcat

SEQ ID NO: 1103 - DNA, 21 bp, Homo sapiens
cctcagccgccgcccctcatc

SEQ ID NO: 1104 - DNA, 21 bp, Homo sapiens
ctcagccgccccccctcatca

SEQ ID NO: 1105 - DNA, 21 bp, Homo sapiens
tcagccgccgcccctcatcat

SEQ ID NO: 1106 - DNA, 21 bp, Homo sapiens
cagccgccgcccctcatcata

SEQ ID NO: 1107 - DNA, 21 bp, Homo sapiens
agccgccgcccctcatcatac

SEQ ID NO: 1108 - DNA, 21 bp, Homo sapiens
gccgccgcccctcatcatacc

SEQ ID NO: 1109 - DNA, 21 bp, Homo sapiens
ccgccgccccccatcatacct

SEQ ID NO: 1110 - DNA, 21 bp, Homo sapiens
cgccgcccctcatcatacctc

SEQ ID NO: 1111 - DNA, 21 bp, Homo sapiens
gccgcccctcctcatacctca

SEQ ID NO: 1112 - DNA, 21 bp, Homo sapiens
ccgcccctcaccatacctcaa

SEQ ID NO: 1113 - DNA, 21 bp, Homo sapiens
cgcccctcatcatacctcaaa

SEQ ID NO: 1114 - DNA, 21 bp, Homo sapiens
gcccctcatcctacctcaaaa

SEQ ID NO: 1115 - DNA, 21 bp, Homo sapiens
cccctcatcacacctcaaaag

SEQ ID NO: 1116 - DNA, 21 bp, Homo sapiens
ccctcatcatccctcaaaagc

SEQ ID NO: 1117 - DNA, 21 bp, Homo sapiens
cgcccctcataatacctcagc

SEQ ID NO: 1118 - DNA, 21 bp, Homo sapiens
gcccctcatcatacctcagcc

SEQ ID NO: 1119 - DNA, 21 bp, Homo sapiens
cccctcatcaaacctcagccg

SEQ ID NO: 1120 - DNA, 21 bp, Homo sapiens
ccctcatcatacctcagccgc

SEQ ID NO: 1121 - DNA, 20 bp, Homo sapiens
tacattgcccatgtaattaa

SEQ ID NO: 1122 - DNA, 20 bp, Homo sapiens
atatagtttcgtcattcatc

SEQ ID NO: 1123 - DNA, 20 bp, Homo sapiens
tacattgcccatgtaattaa

SEQ ID NO: 1124 - DNA, 20 bp, Homo sapiens
atatagtttcgtcattcatc

SEQ ID NO: 1125 - DNA, 20 bp, Homo sapiens
agatagtttagtcattcatc

SEQ ID NO: 1126 - DNA, 20 bp, Homo sapiens
agatagtttcatcattcatc

SEQ ID NO: 1127 - DNA, 20 bp, Homo sapiens
agatagtttcgacattcatc

SEQ ID NO: 1128 - DNA, 20 bp, Homo sapiens
agatagtttcgtaattcatc

SEQ ID NO: 1129 - DNA, 21 bp, Homo sapiens
cctcatcataactcagccgcc

SEQ ID NO: 1130 - DNA, 21 bp, Homo sapiens
ctcatcatacatcagccgccg

SEQ ID NO: 1131 - DNA, 21 bp, Homo sapiens
tcatcataccacagccgccgc

SEQ ID NO: 1132 - DNA, 21 bp, Homo sapiens
catcatacctaagccgccgcc

SEQ ID NO: 1133 - DNA, 21 bp, Homo sapiens
atcatacctcagccgccgccc

SEQ ID NO: 1134 - DNA, 21 bp, Homo sapiens
tcatacctcaaccgccgcccc

SEQ ID NO: 1135 - DNA, 21 bp, Homo sapiens
catacctcagacgccgcccct

SEQ ID NO: 1136 - DNA, 21 bp, Homo sapiens
atacctcagcagccgcccctc

SEQ ID NO: 1137 - DNA, 21 bp, Homo sapiens
tacctcagccaccgcccctca

SEQ ID NO: 1138 - DNA, 21 bp, Homo sapiens
acctcagccgacgcccctcat

SEQ ID NO: 1139 - DNA, 21 bp, Homo sapiens
cctcagccgcagcccctcatc

SEQ ID NO: 1140 - DNA, 21 bp, Homo sapiens
ctcagccgccacccctcatca

SEQ ID NO: 1141 - DNA, 21 bp, Homo sapiens
tcagccgccgaccctcatcat

SEQ ID NO: 1142 - DNA, 21 bp, Homo sapiens
cagccgccgcacctcatcata

SEQ ID NO: 1143 - DNA, 21 bp, Homo sapiens
agccgccgccactcatcatac

SEQ ID NO: 1144 - DNA, 21 bp, Homo sapiens
gccgccgcccatcatcatacc

SEQ ID NO: 1145 - DNA, 21 bp, Homo sapiens
ccgccgccccacatcatacct

SEQ ID NO: 1146 - DNA, 21 bp, Homo sapiens
cgccgcccctaatcatacctc

SEQ ID NO: 1147 - DNA, 21 bp, Homo sapiens
gccgcccctcatcatacctca

SEQ ID NO: 1148 - DNA, 21 bp, Homo sapiens
ccgcccctcaacatacctcaa

SEQ ID NO: 1149 - DNA, 21 bp, Homo sapiens
cgcccctcataatacctcaaa

SEQ ID NO: 1150 - DNA, 21 bp, Homo sapiens
gcccctcatcatacctcaaaa

SEQ ID NO: 1151 - DNA, 21 bp, Homo sapiens
cccctcatcaaacctcaaaag

SEQ ID NO: 1152 - DNA, 21 bp, Homo sapiens
ccctcatcatacctcaaaagc

SEQ ID NO: 1153 - DNA, 21 bp, Homo sapiens
cctcatcatatctcaaaagcc

SEQ ID NO: 1154 - DNA, 20 bp, Homo sapiens
atatagtttcgtcattcatc

SEQ ID NO: 1155 - DNA, 20 bp, Homo sapiens
tacattgcccatgtaattaa

SEQ ID NO: 1156 - DNA, 20 bp, Homo sapiens
atatagtttcgtcattcatc

SEQ ID NO: 1157 - DNA, 20 bp, Homo sapiens
tacattgcccatgtaattaa

SEQ ID NO: 1158 - DNA, 20 bp, Homo sapiens
agatagttttgtcattcatc

SEQ ID NO: 1159 - DNA, 20 bp, Homo sapiens
agatagtttcttcattcatc

SEQ ID NO: 1160 - DNA, 20 bp, Homo sapiens
agatagtttcgtcattcatc

SEQ ID NO: 1161 - DNA, 20 bp, Homo sapiens
agatagtttcgttattcatc

SEQ ID NO: 1162 - DNA, 21 bp, Homo sapiens
ctcatcatacttcaaaagcca

SEQ ID NO: 1163 - DNA, 21 bp, Homo sapiens
tcatcatacctcaaaagccaa

SEQ ID NO: 1164 - DNA, 21 bp, Homo sapiens
catcataccttaaaagccaac

SEQ ID NO: 1165 - DNA, 21 bp, Homo sapiens
atcatacctctaaagccaact

SEQ ID NO: 1166 - DNA, 21 bp, Homo sapiens
tcatacctcataagccaacta

SEQ ID NO: 1167 - DNA, 21 bp, Homo sapiens
catacctcaatagccaactaa

SEQ ID NO: 1168 - DNA, 21 bp, Homo sapiens
atacctcaaatgccaactaac

SEQ ID NO: 1169 - DNA, 21 bp, Homo sapiens
tacctcaaaatccaactaacc

SEQ ID NO: 1170 - DNA, 21 bp, Homo sapiens
acctcaaaagtcaactaacca

SEQ ID NO: 1171 - DNA, 21 bp, Homo sapiens
cctcaaaagctaactaaccaa

SEQ ID NO: 1172 - DNA, 21 bp, Homo sapiens
ctcaaaagcctactaaccaac

SEQ ID NO: 1173 - DNA, 21 bp, Homo sapiens
tcaaaagccatctaaccaacc

SEQ ID NO: 1174 - DNA, 21 bp, Homo sapiens
caaaagccaattaaccaacca

SEQ ID NO: 1175 - DNA, 21 bp, Homo sapiens
aaaagccaactaaccaaccaa

SEQ ID NO: 1176 - DNA, 21 bp, Homo sapiens
aaagccaacttaccaaccaat

SEQ ID NO: 1177 - DNA, 20 bp, Homo sapiens
atatagtttcgtcattcatc

SEQ ID NO: 1178 - DNA, 20 bp, Homo sapiens
tacattgcccatgtaattaa

SEQ ID NO: 1179 - DNA, 20 bp, Homo sapiens
atatagtttcgtcattcatc

SEQ ID NO: 1180 - DNA, 20 bp, Homo sapiens
tacattgcccatgtaattaa

SEQ ID NO: 1181 - DNA, 20 bp, Homo sapiens
agatagttttgtcattcatc

SEQ ID NO: 1182 - DNA, 20 bp, Homo sapiens
agatagtttcttcattcatc

SEQ ID NO: 1183 - DNA, 20 bp, Homo sapiens
agatagtttcgtcattcatc

SEQ ID NO: 1184 - DNA, 20 bp, Homo sapiens
agatagtttcgttattcatc

SEQ ID NO: 1185 - DNA, 21 bp, Homo sapiens
cctcatcatagctcaaaagcc

SEQ ID NO: 1186 - DNA, 20 bp, Homo sapiens
tacattgcccatgtaattaa

SEQ ID NO: 1187 - DNA, 20 bp, Homo sapiens
atatagtttcgtcattcatc

SEQ ID NO: 1188 - DNA, 20 bp, Homo sapiens
tacattgcccatgtaattaa

SEQ ID NO: 1189 - DNA, 20 bp, Homo sapiens
atatagtttcgtcattcatc

SEQ ID NO: 1190 - DNA, 20 bp, Homo sapiens
agatagtttggtcattcatc

SEQ ID NO: 1191 - DNA, 20 bp, Homo sapiens
agatagtttcgtcattcatc

SEQ ID NO: 1192 - DNA, 20 bp, Homo sapiens
agatagtttcggcattcatc

SEQ ID NO: 1193 - DNA, 20 bp, Homo sapiens
agatagtttcgtgattcatc

SEQ ID NO: 1194 - DNA, 21 bp, Homo sapiens
ctcatcatacgtcaaaagcca

SEQ ID NO: 1195 - DNA, 21 bp, Homo sapiens
tcatcataccgcaaaagccaa

SEQ ID NO: 1196 - DNA, 21 bp, Homo sapiens
catcatacctgaaaagccaac

SEQ ID NO: 1197 - DNA, 21 bp, Homo sapiens
atcatacctcgaaagccaact

SEQ ID NO: 1198 - DNA, 21 bp, Homo sapiens
tcatacctcagaagccaacta

SEQ ID NO: 1199 - DNA, 21 bp, Homo sapiens
catacctcaagagccaactaa

SEQ ID NO: 1200 - DNA, 21 bp, Homo sapiens
atacctcaaaggccaactaac

SEQ ID NO: 1201 - DNA, 21 bp, Homo sapiens
tacctcaaaagccaactaacc

SEQ ID NO: 1202 - DNA, 21 bp, Homo sapiens
acctcaaaaggcaactaacca

SEQ ID NO: 1203 - DNA, 21 bp, Homo sapiens
cctcaaaagcgaactaaccaa

SEQ ID NO: 1204 - DNA, 21 bp, Homo sapiens
ctcaaaagccgactaaccaac

SEQ ID NO: 1205 - DNA, 21 bp, Homo sapiens
tcaaaagccagctaaccaacc

SEQ ID NO: 1206 - DNA, 21 bp, Homo sapiens
caaaagccaagtaaccaacca

SEQ ID NO: 1207 - DNA, 21 bp, Homo sapiens
aaaagccaacgaaccaaccaa

SEQ ID NO: 1208 - DNA, 21 bp, Homo sapiens
aaagccaactgaccaaccaat

SEQ ID NO: 1209 - DNA, 20 bp, Homo sapiens
tacattgcccatgtaattaa

SEQ ID NO: 1210 - DNA, 20 bp, Homo sapiens
atatagtttcgtcattcatc

SEQ ID NO: 1211 - DNA, 20 bp, Homo sapiens
tacattgcccatgtaattaa

SEQ ID NO: 1212 - DNA, 20 bp, Homo sapiens
atatagtttcgtcattcatc

SEQ ID NO: 1213 - DNA, 20 bp, Homo sapiens
agatagtttggtcattcatc

SEQ ID NO: 1214 - DNA, 20 bp, Homo sapiens
agatagtttcgtcattcatc

SEQ ID NO: 1215 - DNA, 20 bp, Homo sapiens
agatagtttcggcattcatc

SEQ ID NO: 1216 - DNA, 20 bp, Homo sapiens
agatagtttcgtgattcatc

SEQ ID NO: 1217 - DNA, 21 bp, Homo sapiens
cctcatcatacctcaaaagcc

SEQ ID NO: 1218 - DNA, 20 bp, Homo sapiens
atatagtttcgtcattcatc

SEQ ID NO: 1219 - DNA, 20 bp, Homo sapiens
tacattgcccatgtaattaa

SEQ ID NO: 1220 - DNA, 20 bp, Homo sapiens
atatagtttcgtcattcatc

SEQ ID NO: 1221 - DNA, 20 bp, Homo sapiens
tacattgcccatgtaattaa

SEQ ID NO: 1222 - DNA, 20 bp, Homo sapiens
agatagtttcgtcattcatc

SEQ ID NO: 1223 - DNA, 20 bp, Homo sapiens
agatagtttcctcattcatc

SEQ ID NO: 1224 - DNA, 20 bp, Homo sapiens
agatagtttcgccattcatc

SEQ ID NO: 1225 - DNA, 20 bp, Homo sapiens
agatagtttcgtcattcatc

SEQ ID NO: 1226 - DNA, 21 bp, Homo sapiens
ctcatcatacctcaaaagcca

SEQ ID NO: 1227 - DNA, 21 bp, Homo sapiens
tcatcataccccaaaagccaa

SEQ ID NO: 1228 - DNA, 21 bp, Homo sapiens
catcatacctcaaaagccaac

SEQ ID NO: 1229 - DNA, 21 bp, Homo sapiens
atcatacctccaaagccaact

SEQ ID NO: 1230 - DNA, 21 bp, Homo sapiens
tcatacctcacaagccaacta

SEQ ID NO: 1231 - DNA, 21 bp, Homo sapiens
catacctcaacagccaactaa

SEQ ID NO: 1232 - DNA, 21 bp, Homo sapiens
atacctcaaacgccaactaac

SEQ ID NO: 1233 - DNA, 21 bp, Homo sapiens
tacctcaaaacccaactaacc

SEQ ID NO: 1234 - DNA, 21 bp, Homo sapiens
acctcaaaagccaactaacca

SEQ ID NO: 1235 - DNA, 21 bp, Homo sapiens
cctcaaaagccaactaaccaa

SEQ ID NO: 1236 - DNA, 21 bp, Homo sapiens
ctcaaaagcccactaaccaac

SEQ ID NO: 1237 - DNA, 21 bp, Homo sapiens
tcaaaagccacctaaccaacc

SEQ ID NO: 1238 - DNA, 21 bp, Homo sapiens
caaaagccaactaaccaacca

SEQ ID NO: 1239 - DNA, 21 bp, Homo sapiens
aaaagccaaccaaccaaccaa

SEQ ID NO: 1240 - DNA, 21 bp, Homo sapiens
aaagccaactcaccaaccaat

SEQ ID NO: 1241 - DNA, 20 bp, Homo sapiens
atatagtttcgtcattcatc

SEQ ID NO: 1242 - DNA, 20 bp, Homo sapiens
tacattgcccatgtaattaa

SEQ ID NO: 1243 - DNA, 20 bp, Homo sapiens
atatagtttcgtcattcatc

SEQ ID NO: 1244 - DNA, 20 bp, Homo sapiens
tacattgcccatgtaattaa

SEQ ID NO: 1245 - DNA, 20 bp, Homo sapiens
agatagtttcgtcattcatc

SEQ ID NO: 1246 - DNA, 20 bp, Homo sapiens
agatagtttcctcattcatc

SEQ ID NO: 1247 - DNA, 20 bp, Homo sapiens
agatagtttcgccattcatc

SEQ ID NO: 1248 - DNA, 20 bp, Homo sapiens
agatagtttcgtcattcatc

SEQ ID NO: 1249 - DNA, 21 bp, Homo sapiens
cctcatcataactcaaaagcc

SEQ ID NO: 1250 - DNA, 20 bp, Homo sapiens
tacattgcccatgtaattaa

SEQ ID NO: 1251 - DNA, 20 bp, Homo sapiens
atatagtttcgtcattcatc

SEQ ID NO: 1252 - DNA, 20 bp, Homo sapiens
tacattgcccatgtaattaa

SEQ ID NO: 1253 - DNA, 20 bp, Homo sapiens
atatagtttcgtcattcatc

SEQ ID NO: 1254 - DNA, 20 bp, Homo sapiens
agatagtttagtcattcatc

SEQ ID NO: 1255 - DNA, 20 bp, Homo sapiens
agatagtttcatcattcatc

SEQ ID NO: 1256 - DNA, 20 bp, Homo sapiens
agatagtttcgacattcatc

SEQ ID NO: 1257 - DNA, 20 bp, Homo sapiens
agatagtttcgtaattcatc

SEQ ID NO: 1258 - DNA, 21 bp, Homo sapiens
ctcatcatacatcaaaagcca

SEQ ID NO: 1259 - DNA, 21 bp, Homo sapiens
tcatcataccacaaaagccaa

SEQ ID NO: 1260 - DNA, 21 bp, Homo sapiens
catcatacctaaaaagccaac

SEQ ID NO: 1261 - DNA, 21 bp, Homo sapiens
atcatacctcaaaagccaact

SEQ ID NO: 1262 - DNA, 21 bp, Homo sapiens
tcatacctcaaaagccaacta

SEQ ID NO: 1263 - DNA, 21 bp, Homo sapiens
catacctcaaaagccaactaa

SEQ ID NO: 1264 - DNA, 21 bp, Homo sapiens
atacctcaaaagccaactaac

SEQ ID NO: 1265 - DNA, 21 bp, Homo sapiens
tacctcaaaaaccaactaacc

SEQ ID NO: 1266 - DNA, 21 bp, Homo sapiens
acctcaaaagacaactaacca

SEQ ID NO: 1267 - DNA, 21 bp, Homo sapiens
cctcaaaagcaaactaaccaa

SEQ ID NO: 1268 - DNA, 21 bp, Homo sapiens
ctcaaaagccaactaaccaac

SEQ ID NO: 1269 - DNA, 21 bp, Homo sapiens
tcaaaagccaactaaccaacc

SEQ ID NO: 1270 - DNA, 21 bp, Homo sapiens
caaaagccaaataaccaacca

SEQ ID NO: 1271 - DNA, 21 bp, Homo sapiens
aaaagccaacaaaccaaccaa

SEQ ID NO: 1272 - DNA, 21 bp, Homo sapiens
aaagccaactaaccaaccaat

SEQ ID NO: 1273 - DNA, 20 bp, Homo sapiens
tacattgcccatgtaattaa

SEQ ID NO: 1274 - DNA, 20 bp, Homo sapiens
atatagtttcgtcattcatc

SEQ ID NO: 1275 - DNA, 20 bp, Homo sapiens
tacattgcccatgtaattaa

SEQ ID NO: 1276 - DNA, 20 bp, Homo sapiens
atatagtttcgtcattcatc

SEQ ID NO: 1277 - DNA, 20 bp, Homo sapiens
agatagtttagtcattcatc

SEQ ID NO: 1278 - DNA, 20 bp, Homo sapiens
agatagtttcatcattcatc

SEQ ID NO: 1279 - DNA, 20 bp, Homo sapiens
agatagtttcgacattcatc

SEQ ID NO: 1280 - DNA, 20 bp, Homo sapiens
agatagtttcgtaattcatc

SEQ ID NO: 1281 - DNA, 39 bp, Homo sapiens
gttttcccagtcacgacttggttggttattagagggtgg

SEQ ID NO: 1282 - DNA, 38 bp, Homo sapiens
aaacagctatgaccatgaccataaccaaccaatcaacc

SEQ ID NO: 1283 - DNA, 144 bp, Homo sapiens
ctggctggtcaccagagggtggggcggaccgagtgcgctcggcggctgcggagaggggtagagcaggcagcgggcggcggggagcagcatggagccggcggcggggagcagcatggagccttcggctgactggctggccacggc

SEQ ID NO: 1284 - DNA, 18 bp, Homo sapiens
ttagaggatttgagggat

SEQ ID NO: 1285 - DNA, 18 bp, Homo sapiens
aaaactccatactactcc

SEQ ID NO: 1286 - DNA, 60 bp, Homo sapiens
cttggctgtcccagaatgcaagaagcccagacggaaaccgtagctgccctggtaggtttt

SEQ ID NO: 1287 - DNA, 20 bp, Homo sapiens
tatatcaaagcagtaagtag

SEQ ID NO: 1288 - DNA, 90 bp, Homo sapiens
ccaccctctaataaccaaccaacccctcctctttcttcctccaatactaacaaaaaaaccccctccaaccctatccctcaaatcctctaa

SEQ ID NO: 1289 - DNA, 90 bp, Homo sapiens
gtgtgtttggtggttgcggagagggggagagtaggtagtgggtggtggggagtagtatggagttggtggtggggagtagtatggagtttt

SEQ ID NO: 1290 - DNA, 100 bp, Homo sapiens
ttagaggatttgagggatagggttggagggggtttttttgttagtattggaggaagaaagaggaggggttggttggttattagagggtggggtggattgt

SEQ ID NO: 1291 - DNA, 100 bp, Homo sapiens
aaaactccatactactccccaccaccaactccatactactccccaccacccactacctactctccccctctccgcaaccaccaaacacacacaatccacc

(19) United States
(12) Patent Application Publication
(10) Pub. No.: US 2003/0152950 A1
(43) Pub. Date: Aug. 14, 2003

(54) Identification of chemically modified polymers

(76) Inventors: Harold R. Garner, Coppell, TX (US); John D. Minna, Dallas, TX (US); Kevin J. Luebke, Dallas, TX (US); Robert P. Balog, Dallas, TX (US)

Correspondence Address: Sanford E. Warren, Gardere Wynne Sewell LLP, Suite 3000, 1601 Elm Street, Dallas, TX, 75201-4761, US

(21) Appl. No.: 10/184,085

(22) Filed: Jun. 27, 2002

Related U.S. Application Data
(60) Provisional application No. 60/301,370, filed on Jun. 27, 2001.

(51) Int. Cl. C12Q 1/68

(52) U.S. Cl. 435/6

(57) ABSTRACT

The present invention provides a high-throughput method for the parallel analysis of many potential sites of chemical modification (e.g., methylation) in DNA. It makes use of chemical treatment of the DNA to alter its sequence in a way that depends upon the modification of interest and subsequent analysis of the resulting sequence by hybridization to an array of probes. A device, comprising the array of probes, is provided by the invention, and principles and methods for its design and fabrication are also provided.

This application claims priority to U.S. Provisional Patent Application Serial No. 60/301,370 filed Jun. 27, 2001.

The U.S. Government may own certain rights in this invention pursuant to the terms of the National Cancer Institute grant CA81656-01.

### FIELD OF THE INVENTION

The present invention relates generally to the analysis of chemically modified macromolecules, and specifically to the detection of modified sites in DNA with the use of oligonucleotide arrays.

### BACKGROUND OF THE INVENTION

Methylation of cytosines in CpG dinucleotides is an important mechanism of transcriptional regulation. It is involved in a variety of normal biological processes such as X chromosome inactivation and transcriptional regulation of imprinted genes. Aberrant methylation of cytosines can also effect transcriptional inactivation of certain tumor suppressor genes, associated with a number of human cancers. Cytosine methylation in CpG-rich areas (CpG islands) located in the promoter regions of some genes is of special regulatory importance. Therefore, wide scope mapping of methylation sites in CpG islands is important for understanding both normal and pathological cellular processes. Furthermore, methylation of certain sites may serve as an important marker for early diagnosis and treatment decisions of some cancers.

A variety of methods have been used to identify sites of DNA methylation. One common method has relied on the inability of restriction endonucleases to cleave sequences that contain one or more methylated cytosines. Genomic DNA is fragmented with appropriate restriction enzymes and cleavage at the site of interest is probed electrophoretically or by PCR. This method provides an analysis of some potential methylation sites, but it is limited to sites that fall within the recognition sequences of methylation-sensitive restriction enzymes.

Other methods rely on the differential chemical reactivities of cytosine and 5-methyl cytosine with reagents such as sodium bisulfite, hydrazine, or permanganate. In the case of hydrazine and permanganate, differential strand cleavage between methylated and unmethylated cytosines is examined in a similar fashion to that used when cleavage is done with restriction enzymes. This approach is complicated by the imperfect specificity of the reagents between methylated and unmethylated cytosines and by interference from reaction with thymidines.

Treatment with sodium bisulfite can be used to convert methylated and unmethylated DNA to different sequences. Under appropriate conditions, unmethylated cytosines in DNA react with sodium bisulfite to yield deoxyuridine, which behaves as thymidine in Watson-Crick hybridization and enzymatic template-directed polymerization. Methylated cytosines, however, are unreactive, and behave as cytosine in Watson-Crick hybridization and enzymatic template-directed polymerization.

The sequence differences resulting from bisulfite treatment can be assessed in any of several ways. One way is with standard sequencing by primer extension (Sanger sequencing). This method has the disadvantage of limited throughput. Another way, termed methylation-specific PCR, uses a set of PCR primers specific to the sequences resulting from bisulfite treatment of either methylation state at a given site. Effective amplification using one primer from the set indicates methylation, whereas effective amplification using the other primer indicates unmethylated cytosine at the site being amplified. This method has the disadvantage of low sample throughput in addition to the disadvantage that only one potential site of methylation is probed in an assay.

Thus, there is a need for a high throughput method for the identification of alteration in DNA.

### SUMMARY OF THE INVENTION

The present invention provides a high-throughput method for the parallel analysis of many potential sites of chemical modification (e.g., methylation) in DNA. It makes use of chemical treatment of the DNA to alter its sequence in a way that depends upon the modification of interest and subsequent analysis of the resulting sequence by hybridization to an array of probes. A device, comprising the array of probes, is provided by the invention, and principles and methods for its design and fabrication are also provided.

In one form the present is a method for the analysis of chemical modification of DNA including the steps of obtaining a sample of DNA to be analyzed and treating the DNA with one or more chemical reagents that result in different base sequences depending upon the presence or absence of the modification of interest, and determining a portion of the base sequence of the resulting DNA.

Another form of the present invention is an array of one or more nucleic acid probes immobilized on a solid support wherein the probes are designed to detect sites of methylation in DNA.

Yet another form of the invention is a method for generating DNA probe sequences that includes the steps of inputting a nucleic acid sequence in the 3-prime to 5-prime direction and converting the sequence to account for chemical modification. The complementary sequence to the converted sequence in the 3-prime to 5-prime direction is then generated. A first parent probe is then generated by choosing a first starting position on the complementary sequence and a first ending position on the complementary sequence. A second parent probe is then generated by moving the first starting and first ending position one base unit in the same direction. This process may be repeated as often as desired.

Another form of the resent invention is a method for generating DNA probe sequences that includes the steps of inputting a nucleic acid sequence in the 3-prime to 5-prime direction and converting the sequence to account for chemical modification. The complementary sequence to the converted sequence in the 3-prime to 5-prime direction is then generated. The complementary sequence is then examined to locate one or more CpG dinucleotide regions within the complementary sequence, and probes are then generated that have one or more nucleic acid bases on each end of the CpG dinucleotide regions.

### BRIEF DESCRIPTION OF THE FIGURES

The above and further advantages of the invention may be better understood by referring to the following detailed description in conjunction with the accompanying drawings in which corresponding numerals in the different FIGURES refer to the corresponding parts in which:

FIG. 1 depicts a reaction in accordance with the present invention;

FIG. 2 depicts a method of re-sequencing in accordance with the present invention;

FIG. 3 depicts a schematic of assay results in accordance with the present invention;

FIG. 4 depicts the results of a two-color assay in accordance with the present invention;

FIG. 5 depicts a fluorescence scan in accordance with the present invention;

FIG. 6 depicts an assay for CpG methylation by (A) treatment with sodium bisulfite to convert unmethylated cytosines to deoxyuracils (4 cytosines) while methylated cytosines remain unconverted (one cytosine denoted as methylated with a superscript Me) and (B) sequence analysis of a labeled representative of the bisulfite-treated DNA by hybridization to an array of oligonucleotides in accordance with the present invention;

FIG. 7 depicts the sequence of the 190 base region of the p16 promoter wherein each cytosine in the sequence is numbered in accordance with the present invention;

FIG. 8 depicts four probes from an array used to analyze the methylation state of a region of the promoter for p16 showing (A) fluorescence scan of the Cy5 (analyte) channel of the array, (B) fluorescence scan of the Cy3 (reference) channel of the array, (C) overlay of the analyte and reference channels demonstrating the appearance of a methylated site compared with an unmethylated reference in accordance with the present invention; and

FIG. 9 is a histogram plots showing Z scores for each cytosine in a CpG dinucleotide using analysis in which the analyte was derived from (A) uniformly methylated DNA, (B) a synthetic duplex simulating unique methylation at cytosine number 25, (C) a mixture of approximately 20% methylated DNA and 80% unmethylated DNA in accordance with the present invention.

### DETAILED DESCRIPTION

While the making and using of various embodiments of the present invention are discussed herein in terms of identification of methylated sites in DNA, it should be appreciated that the present invention provides many applicable inventive concepts that can be embodied in a wide variety of specific contexts. The specific embodiments discussed herein are merely illustrative of specific ways to make and use the invention and are not meant to limit the scope of the invention in any manner.

The need for high-throughput methods is highlighted by the prevalence of CpG islands in the genome. Computer analysis of the March 2001 Unigene build reveals 32,597 of the 92,152 clusters contain CpG islands. Of the 14,968 clusters with annotation, 10,438 have CpG islands. These islands in the annotated clusters comprise 4,398,560 bp in 5′ non-coding regions, 7,074,411 bp in coding regions, and 492,323 bp in 3′ non-coding regions. A high throughput method of the present invention will be necessary to interrogate even a small fraction of these sites in a given experiment.

The differential reactivity of bisulfite with cytosine and 5-methylcytosine forms the basis of several techniques for the assessment of DNA methylation; however, new approaches to the read-out of the sequence that results from treatment with bisulfite are desirable. Sequence analysis by hybridization to oligonucleotide arrays is an approach that affords a high degree of parallelism and flexibility. The present invention relies on discrimination between a cytosine and a thymidine in the array hybridization.

All technical and scientific terms used herein have the same meaning as commonly understood by one of ordinary skill in the art to which this invention belongs, unless defined otherwise. Methods and materials similar or equivalent to those described herein may be used in the practice or testing of the present invention, the generally used methods and materials are now described.

Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used throughout the present specification the following abbreviations are used: TF, transcription factor; ORF, open reading frame; kb, kilobase (pairs); UTR, untranslated region; kD, kilodalton; PCR, polymerase chain reaction; RT, reverse transcriptase.

The term “homology” refers to the extent to which two nucleic acids are complementary. There may be partial or complete homology. A partially complementary sequence is one that at least partially inhibits a completely complementary sequence from hybridizing to a target nucleic acid and is referred to using the functional term “substantially homologous.” The degree or extent of hybridization may be examined using a hybridization or other assay (such as a competitive PCR assay) and is meant, as will be known to those of skill in the art, to include specific interaction even at low stringency.

The art knows that numerous equivalent conditions may be employed to achieve low stringency conditions. Factors that affect the level of stringency include: the length and nature (DNA, RNA, base composition) of the probe and nature of the target (DNA, RNA, base composition, present in solution or immobilized, etc.) and the concentration of the salts and other components (e.g., formamide, dextran sulfate, polyethylene glycol). Likewise, the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, inclusion of formamide, etc.).

The term “gene” is used to refer to a functional protein, polypeptide or peptide-encoding unit. As will be understood by those in the art, this functional term includes genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

The term “portion of a genome for genetic analysis” or “chromosome-specific” is herein defined to encompass the terms “target specific” and “region specific”, that is, when the staining composition is directed to one chromosome or portion of a genome, it is chromosome-specific, but it is also chromosome-specific when it is directed, for example, to multiple regions on multiple chromosomes, or to a region of only one chromosome, or to regions across the entire genome. Likewise, “locus specific” or “loci specific” is defined as locations on one or more chromosomes for a particular gene or allele. Sequence from regions of one or more chromosomes are sources for probes for that region or those regions of the genome. The probes produced from such source material are region-specific probes but are also encompassed within the broader phrase “portion of a genome” probes. The term “target specific” is interchangeably used herein with the term “chromosome-specific” and “portion of a genome”.

The word “specific” as commonly used in the art has two somewhat different meanings. The practice is followed herein. “Specific” refers generally to the origin of a nucleic acid sequence or to the pattern with which it will hybridize to a genome, e.g., as part of a staining reagent. For example, isolation and cloning of DNA from a specified chromosome results in a “chromosome-specific library.” Shared sequences are not chromosome-specific to the chromosome from which they were derived in their hybridization properties since they will bind to more than the chromosome of origin. A sequence is “locus specific” if it binds only to the desired portion of a genome. Such sequences include single-copy sequences contained in the target or repetitive sequences, in which the copies are contained predominantly in the selected sequence.

A “probe” as defined herein may be one or more molecules that can hybridize to a nucleic acid target sequence and that can be detected (e.g., nucleic acid fragments or other oligomers that bind nucleic acids). Examples of possible probe molecules include, but are not limited to, DNA, RNA, peptides, minor groove-binding polyamides, peptide nucleic acids (PNA), locked nucleic acids (LNA), and 2′-O-methyl nucleic acids. The probe is labeled so that its binding to the target can be assayed, visualized or detected. In essence the probe is designed to bind a target, also referred to as an analyte, so that the combination of probe and analyte may be assayed, visualized or detected. The probe may be produced from some source of nucleic acid sequences, for example, a collection of clones or a collection of polymerase chain reaction (PCR) products or the product of nick translation or other methods for adding a detectable marker to a nucleic acid binding moiety. For nucleic acids, repetitive sequences are removed or blocked with unlabeled nucleic acid with complementary sequence, so that hybridization with the resulting probe produces staining of sufficient contrast on the target. The word probe may be used herein to refer not only to a molecule that detects a nucleic acid, but also to the detectable nucleic acid in the form in which it is applied to, e.g., the surface of an array. What “probe” refers to specifically should be clear to those of skill in the art from the context in which the word is used.

The term “labeled” as used herein indicates that there is some method to visualize or detect the bound probe, whether or not the probe directly carries some modified constituent. The terms “staining” or “painting” are herein defined to mean hybridizing a probe of this invention to a genome or segment thereof, such that the probe reliably binds to the targeted region or sequence of chromosomal material and the bound probe is capable of being detected. The terms “staining” or “painting” are used interchangeably. The patterns on the array resulting from “staining” or “painting” are useful for cytogenetic analysis, more particularly, molecular cytogenetic analysis. The staining patterns facilitate the high-throughput identification of normal and abnormal chromosomes and the characterization of the genetic nature of particular abnormalities.

Multiple methods of probe detection may be used with the present invention, e.g., the binding patterns of different components of the probe may be distinguished, for example, by color or differences in wavelength emitted from a labeled probe.

A number of different aberrations may be detected with any desired staining pattern on the portions of the genome detected with one or more colors (a multi-color staining pattern) and/or other indicator methods.

The complexity for a final probe list and array will depend on the application for which it is designed (e.g., location on the genome, complexity of the sequence, etc.) and the mapping resolution that is sought. In general, the larger the target area, the more complex the probe list. The term “complexity” therefore refers to the complexity of the total probe list no matter how many visually distinct loci are to be detected, that is, regardless of the distribution of the target sites over the genome.

The required contrast (e.g., signal to noise) for detection will depend on the application for which the probe is designed and even the portion of the genome that is the target of the analysis. When visualizing chromosomes and nuclei, etc., microscopically, a contrast ratio of two or greater is often sufficient for identifying whole chromosomes. When quantifying the amount of target region present on an array by fluorescence intensity measurements using a slide reader or quantitative microscopy.

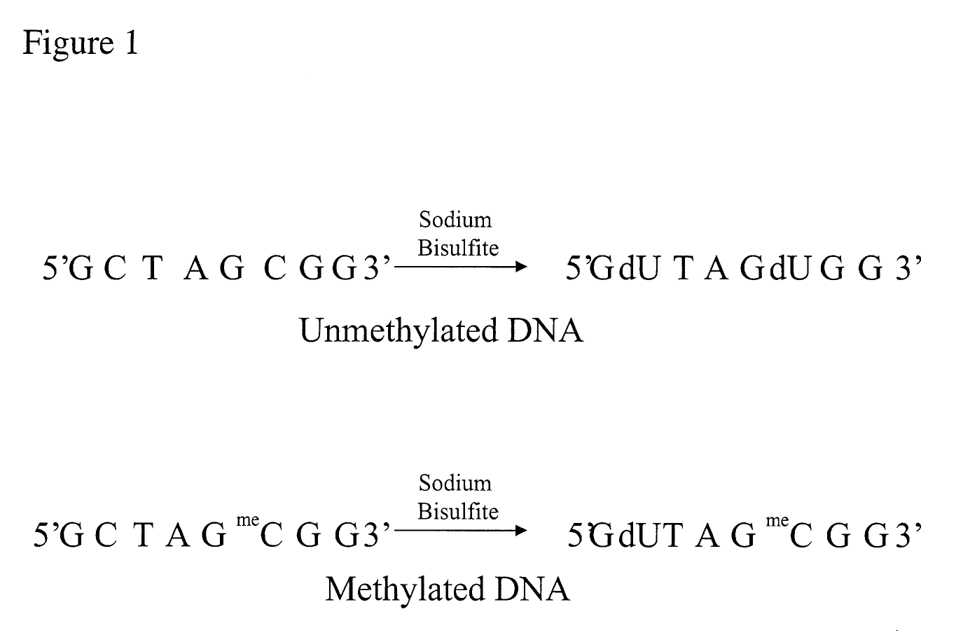

Identification of a large number of individual methylation sites in a high-throughput, highly parallel assay can be accomplished by specifically converting only unmethylated cytosines to deoxyuridines with sodium bisulfite treatment, as shown in FIG. 1, and rapidly reading out the resulting sequence. Any cytosine remaining in the product is identified as a site of methylation. Oligonucleotide arrays are particularly well suited to rapidly distinguishing between closely related nucleic acid sequences with a method known as re-sequencing.

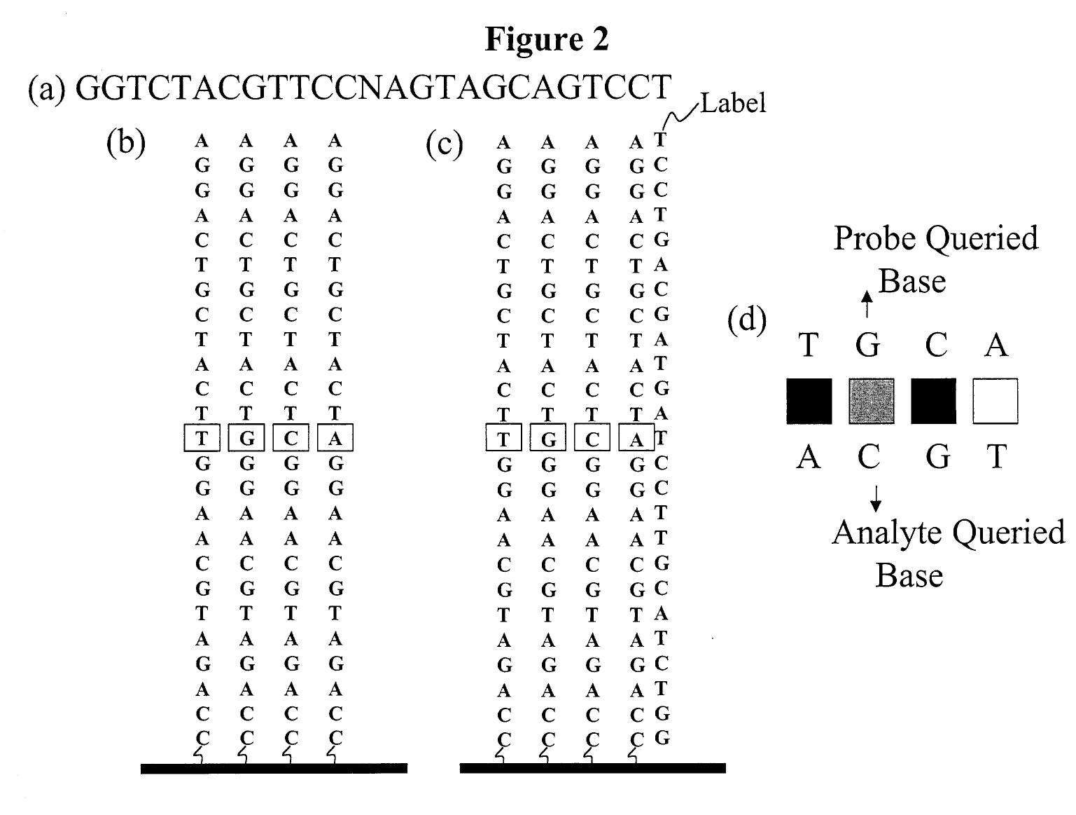

The method of re-sequencing is depicted in FIG. 2. A sequence of interest is shown in FIG. 2A, where an unknown base is at a central position, identified in the FIGURE with an N. FIG. 2B shows four oligonucleotide probes used to assay each base position of interest, each probe complementary to the sequence being tested except at the position of the unknown base. At the position of the unknown base, the probes differ, each having a different one of the four possible bases. The probe oligonucleotides may be immobilized on a surface as shown in FIG. 2, but other formats are possible. FIG. 2C shows the DNA to be tested binding to one of the four probes. It binds specifically to the probe with an adenosine in the test position, identifying the unknown base, N, as a thymidine. Specificity is highest when the probed base binds near the center of probe oligonucleotide.

In practice, re-sequencing with oligonucleotide arrays can be accomplished by a number of means, any of which will be applicable to the present invention. In one standard approach, the array of oligonucleotides is immobilized on a glass surface. An example of a “feature” of the resulting array is defined as a region of the surface in which a single probe sequence predominates. Fabrication of surface-bound oligonucleotide arrays can also be accomplished by a variety of methods known to those with skill in the art.

A fabrication method that is particularly appropriate for the present invention makes use of light directed chemistry to synthesize the oligonucleotides directly on the surface. The regions of the surface that are illuminated during pre-determined chemical steps of the synthesis determine the sequence synthesized in each feature. Defined regions can be illuminated discretely by, for example, shining light through a physical mask that blocks light from particular regions or by directing light to particular regions with a digital micromirror array. These light-directed approaches are desirable for the present invention, because they currently enable the largest numbers of features per unit area of array surface. Thus, the potential of the current invention for highly parallel analysis of methylation is best met by the very high feature numbers accessible with light-directed methods. However, other methods of array fabrication are amenable to the present invention, including but not limited to delivering the reagents of DNA synthesis to specific regions of the surface and depositing on the surface oligonucleotides that have been pre-synthesized.

Typically, a solution of the nucleic acid to be analyzed is applied to the surface of the array, and the dissolved nucleic acid is allowed to bind to probes on the surface. After an appropriate time, the unbound and the weakest bound nucleic acid are washed from the array and the bound nucleic acid is detected. Detection of binding can be accomplished in several ways known to those of skill in the art, any of which can be applied to the present invention. In one method, detection is accomplished by labeling the test nucleic acid with a moiety such as a fluorophore and measuring fluorescence associated with each probe. FIG. 2D schematically illustrates the appearance of a fluorescence scan of four features designed to probe a single base following binding and washing. The brightest feature indicates the identity of the probed base position. Many methods are also known for the incorporation of a fluorescent label into a test nucleic acid, including but not limited to nick translation, transcription into RNA using a template-directed RNA polymerase to incorporate labeled nucleotide triphosphates, or amplifying a region of interest with PCR using labeled primers.

In operation, the present invention may be used, for example, as described herein. A sample of genomic DNA to be analyzed is obtained and treated with bisulfite under conditions for which that reaction converts unmethylated cytosines to deoxyuridines but does not effect methylated cytosines. One or more regions of interest from the resulting DNA are then amplified by PCR and labeled by any of a variety of methods. Design of primers for PCR amplification of bisulfite-treated DNA should be guided by the following considerations: 1) the primers should not contain CpG dinucleotides of unknown methylation state, 2) the primers are restricted to a three-base code (A, G, and T) because all cytosines not in CpG dinucleotides are converted to deoxyuridine, 3) some bisulfite treatment protocols, such as the one described below, cleave the DNA substantially, so amplification of short regions (about 200 base pairs) is most successful, and 4) a different set of primers is required for each strand, because the two initially complementary strands are no longer complementary after bisulfite treatment.

A solution of the labeled nucleic acid is then contacted with an array of probes comprising probes that bind differentially to the sequences resulting from bisulfite treatment of methylated or unmethylated cytosines of interest. In practice, such probes can be made by creating oligonucleotides that are complementary to a region of DNA surrounding the cytosine of interest, taking into account the conversion of all cytosines not in a CpG dinucleotide to deoxyuridine, which is complementary to adenosine. A typical length for such oligonucleotide probes is between 15 and 30 nucleotides, but longer and shorter probes are possible. The site to be probed should be near the center of the region to which the probe is complementary.

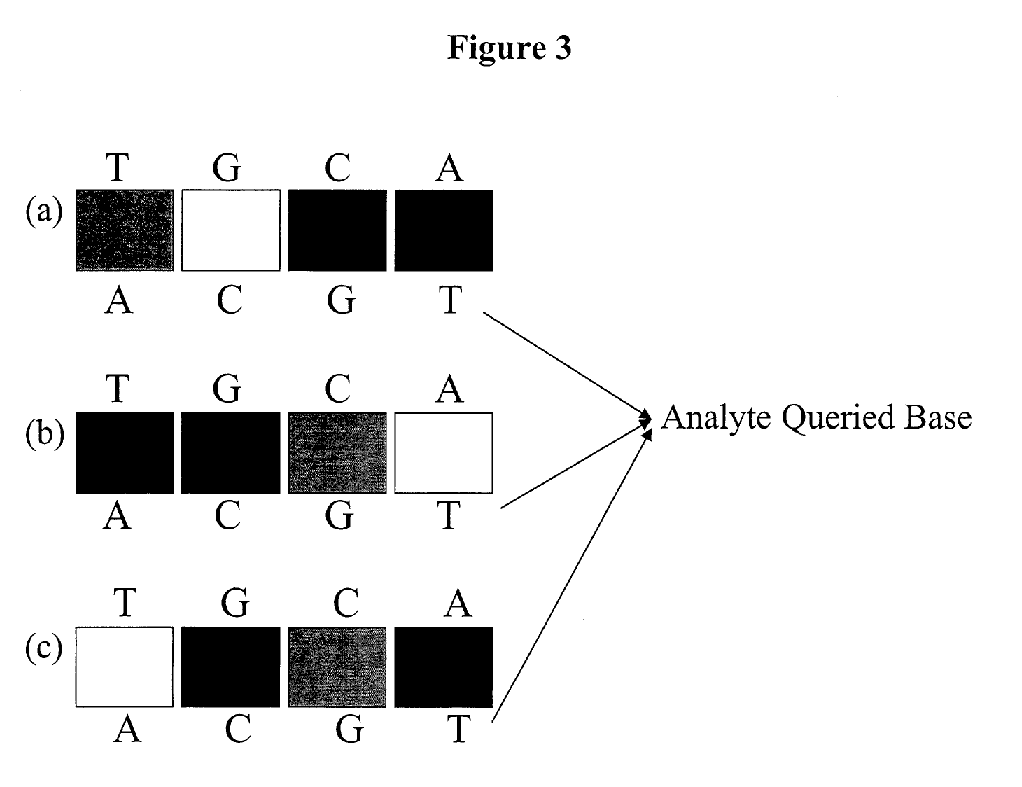

At least two probes are required for each potential methylation site of interest. In one, the base in apposition to the site to be probed is an adenosine, forming the complement to the deoxyuridine-containing sequence corresponding to the unmethylated state. In the other, the base at the same position is guanosine, forming the complement to the cytosine-containing sequence corresponding to the methylated state. Although methylation state can be determined with these two probes only, it is preferable to use four probes for every site, one with each of the four bases at he variable position, in order to account for the possibility of polymorphism or mutation at the site of interest. Possible results of this assay are shown schematically in FIG. 3. FIG. 3A illustrates a result indicating methylation of the site of interest, the brightest feature being that corresponding to cytosine. FIG. 3B illustrates a result indicating absence of methylation at the site of interest, the brightest feature being that corresponding to thymidine. FIG. 3C illustrates a result indicating polymorphism or mutation at the site of interest to an adenosine.

Multiple CpG dinucleotides of unknown methylation state will often be sufficiently proximal to each other in sequences to be analyzed that the probe will include one or more CpG dinucleotides in addition to the central one being analyzed. If a methylation state is assumed for these additional sites in the design of the probe sequence, the probe affinity for the analyte will be diminished whenever the assumed methylation state is not the actual methylation state. Including on the array additional probes that accommodate all possible methylation states can compensate for the resulting decrease in signal.

The array may comprise probes that have been selected by visual inspection of the sequences to be probed or probes that have been selected by automated computational means. Because the present invention is most advantageous when probing a large number of sites in parallel, the preferred method of probe choice is by automated computational means. A process for probe selection is outlined below. Automated searching of genome databases can identify regions of particular interest with a high density of CpG dinucleotides.

Two or more labels, such as fluorophores with different excitation and emission frequencies, can be used to compare one or more test samples with a reference sample. The reference sample can be a standard of known methylation state, a DNA sample from a reference tissue, such as a healthy tissue proximal to a diseased tissue to be tested, or a sample from the same cellular source as the test sample that has not been treated with bisulfite. The use of a reference sample of known methylation state provides an internal control for expected relative binding to probes, resulting in higher confidence in assignment of methylation state of unknown samples. The use of a reference sample from a reference tissue provides facile identification of methylation that is related to a particular phenotype, such as a disease phenotype. The use of a reference sample from the same cellular source as the test sample provides control for the possibility of a cytosine to thymidine mutation or polymorphism.

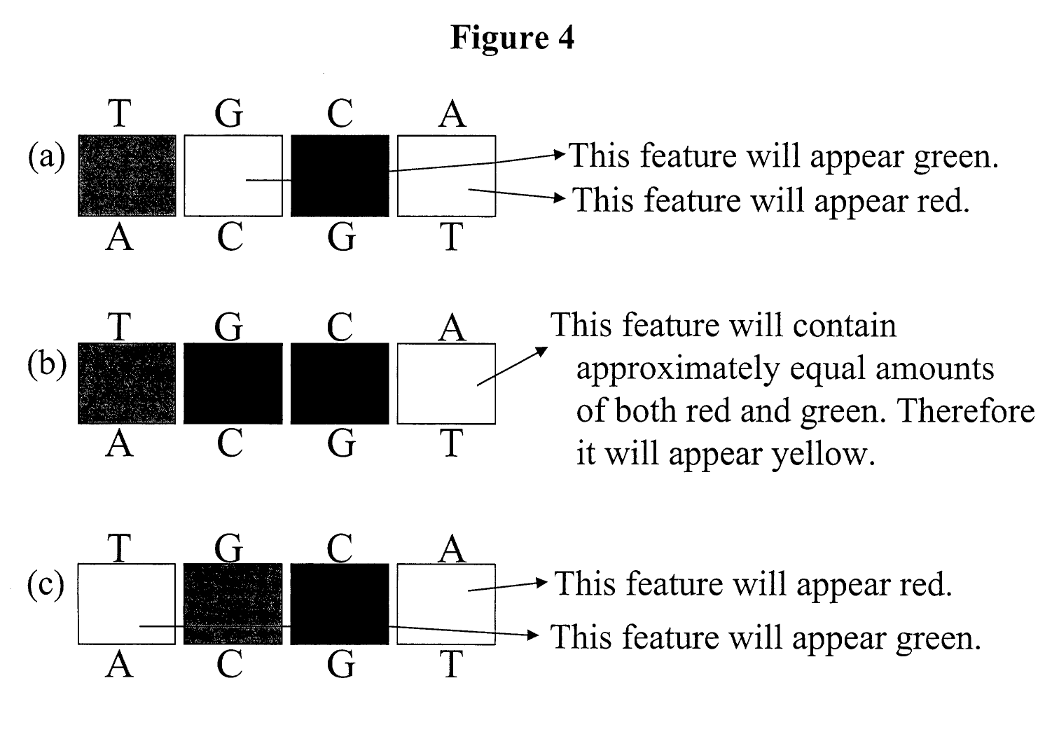

Possible results of a two-color assay with an unmethylated reference sample are shown in FIG. 4. The reference sample is labeled with the red dye, and the sample to be analyzed is labeled with the green dye. FIG. 4A illustrates a result indicating methylation of the site of interest, the brightest green feature being that corresponding to cytosine and the brightest red feature corresponding to thymidine. FIG. 4B illustrates a result indicating absence of methylation at the site of interest, the brightest feature in both data channels being that corresponding to thymidine. FIG. 4C illustrates a result indicating polymorphism or mutation at the site of interest to an adenosine.

The probes of the array need not be restricted to DNA. Any molecule that binds differentially to the sequences resulting from bisulfite treatment of methylated and unmethylated DNA can be used. Examples of possible probe molecules include, but are not limited to, RNA, peptides, minor groove-binding polyamides, peptide nucleic acids (PNA), locked nucleic acids (LNA), and 2′-O-methyl nucleic acid.

### EXAMPLE 1

### Analysis of Methylation of a Region of the Promoter for the Tumor Suppressor Gene p16

Genomic DNA was isolated from two lines of lung tumor cells, H69 and H1618. The promoter region of the tumor suppressor gene P16 is known to be methylated at cytosines in CpG dinucleotides in the line H1618 and is not methylated in the line H69. DNA from both lines was treated with sodium bisulfite as described in the protocol below, which converts unmethylated cytosine to deoxyuridine (essentially equivalent to thymidine in hybridization) but does not react with methylated cytosine. A 145 base pair region from the p16 promoter from each cell line was amplified with labeled primers. Primers labeled with Cy5 were used to amplify the unmethylated promoter (which represents a control or reference sequence) and primers labeled with Cy3 were used to amplify the methylated promoter (which represents the unknown methylation state to be analyzed).

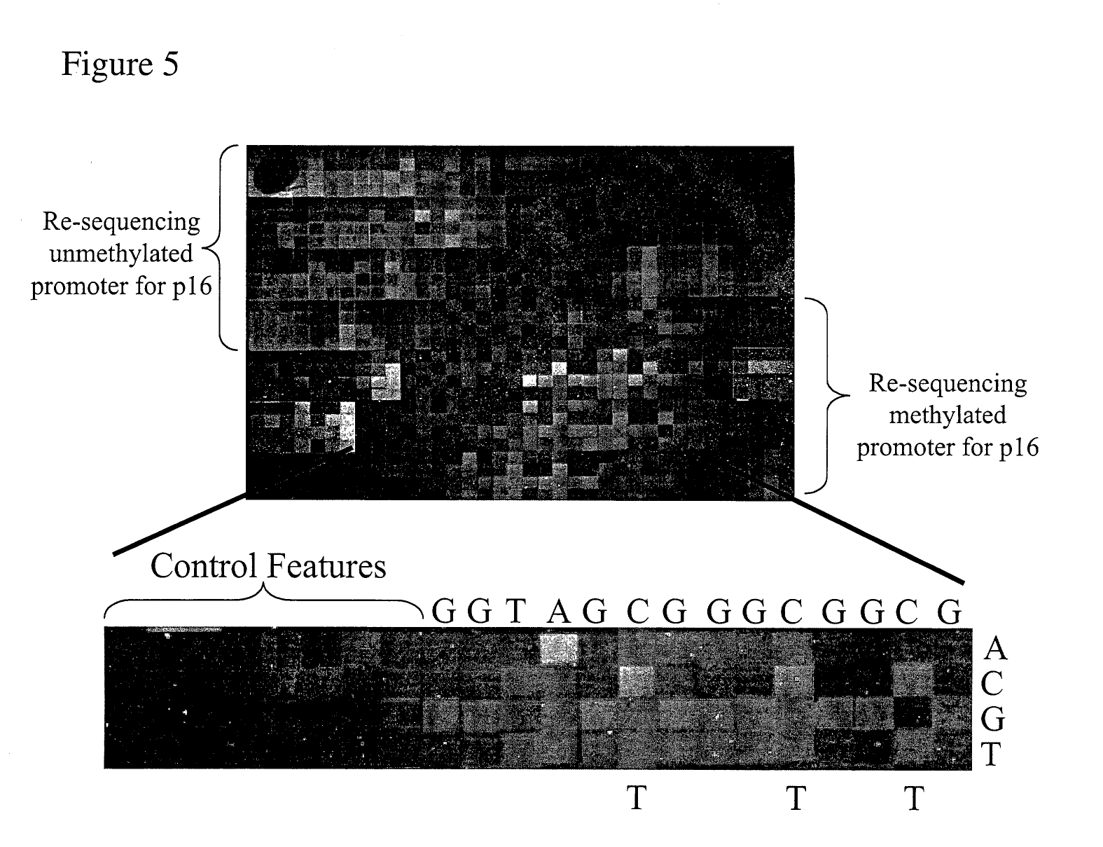

The two samples were mixed together with the labeled control oligonucleotide and applied to the array. The array, fabricated by light-directed chemistry using a digital micromirror array, had two sets of features in addition to the control features. One set of features (upper half of array) was a standard re-sequencing tiling for the sequence expected without methylation (i.e., all Cs converted to T). The other set was a standard re-sequencing tiling for the sequence expected with methylation of every C in each CpG step. The set of probes used in the array appears as TABLE 1. A two-color fluorescence scan of the array after hybridization for 16 hours at room temperature and washing with 1×SSPE is shown in FIG. 5. Overall methylation state is evident by the labeled sample which binds best to each set of features, the Cy5 labeled, unmethylated sample binding best to the upper tiles for unmethylated sequence (highest signal red) and the Cy3 labeled, methylated sample binding best to the lower tiles for methylated sequence (highest signal green). Specific sites of methylation can be observed by reading sequence directly and by visually identifying columns in which the feature for C is green and the feature for T is red (easily visualized in both sets of probes).

TABLE 1
Probes Used in the Array

| SEQ ID NO | Nucleotide Sequence for Probe |
|---|---|
| SEQ ID NO: 1 | AACCAACCAATAATCTCCCAC |
| SEQ ID NO: 2 | ACCAACCAATTATCTCCCACC |
| SEQ ID NO: 3 | CCAACCAATATTCTCCCACCC |
| SEQ ID NO: 4 | CAACCAATAATCTCCCACCCC |
| SEQ ID NO: 5 | AACCAATAATTTCCCACCCCA |
| SEQ ID NO: 6 | ACCAATAATCTCCCACCCCAC |
| SEQ ID NO: 7 | CCAATAATCTTCCACCCCACC |
| SEQ ID NO: 8 | CAATAATCTCTCACCCCACCT |
| SEQ ID NO: 9 | AATAATCTCCTACCCCACCTA |
| SEQ ID NO: 10 | ATAATCTCCCTCCCCACCTAA |
| SEQ ID NO: 11 | TAATCTCCCATCCCACCTAAC |
| SEQ ID NO: 12 | AATCTCCCACTCCACCTAACT |
| SEQ ID NO: 13 | ATCTCCCACCTCACCTAACTC |
| SEQ ID NO: 14 | TCTCCCACCCTACCTAACTCA |
| SEQ ID NO: 15 | CTCCCACCCCTCCTAACTCAC |
| SEQ ID NO: 16 | TCCCACCCCATCTAACTCACA |
| SEQ ID NO: 17 | CCCACCCCACTTAACTCACAC |
| SEQ ID NO: 18 | CCACCCCACCTAACTCACACA |
| SEQ ID NO: 19 | CACCCCACCTTACTCACACAA |
| SEQ ID NO: 20 | ACCCCACCTATCTCACACAAA |
| SEQ ID NO: 21 | CCCCACCTAATTCACACAAAC |
| SEQ ID NO: 22 | CCCACCTAACTCACACAAACC |
| SEQ ID NO: 23 | CCACCTAACTTACACAAACCA |
| SEQ ID NO: 24 | CACCTAACTCTCACAAACCAC |
| SEQ ID NO: 25 | ACCTAACTCATACAAACCACC |
| SEQ ID NO: 26 | ATATAGTTTCGTCATTCATC |
| SEQ ID NO: 27 | TACATTGCCCATGTAATTAA |
| SEQ ID NO: 28 | ATATAGTTTCGTCATTCATC |
| SEQ ID NO: 29 | TACATTGCCCATGTAATTAA |
| SEQ ID NO: 30 | AGATAGTTTTGTCATTCATC |
| SEQ ID NO: 31 | AGATAGTTTCTTCATTCATC |
| SEQ ID NO: 32 | AGATAGTTTCGTCATTCATC |
| SEQ ID NO: 33 | AGATAGTTTCGTTATTCATC |
| SEQ ID NO: 34 | CCTAACTCACTCAAACCACCA |
| SEQ ID NO: 35 | CTAACTCACATAAACCACCAA |
| SEQ ID NO: 36 | TAACTCACACTAACCACCAAC |
| SEQ ID NO: 37 | AACCAACCAAGAATCTCCCAC |
| SEQ ID NO: 38 | ACCAACCAATGATCTCCCACC |
| SEQ ID NO: 39 | CCAACCAATAGTCTCCCACCC |
| SEQ ID NO: 40 | CAACCAATAAGCTCCCACCCC |
| SEQ ID NO: 41 | AACCAATAATGTCCCACCCCA |
| SEQ ID NO: 42 | ACCAATAATCGCCCACCCCAC |
| SEQ ID NO: 43 | CCAATAATCTGCCACCCCACC |
| SEQ ID NO: 44 | CAATAATCTCGCACCCCACCT |
| SEQ ID NO: 45 | AATAATCTCCGACCCCACCTA |
| SEQ ID NO: 46 | ATAATCTCCCGCCCCACCTAA |
| SEQ ID NO: 47 | TAATCTCCCAGCCCACCTAAC |
| SEQ ID NO: 48 | AATCTCCCACGCCACCTAACT |
| SEQ ID NO: 49 | ATCTCCCACCGCACCTAACTC |
| SEQ ID NO: 50 | TCTCCCACCCGACCTAACTCA |
| SEQ ID NO: 51 | CTCCCACCCCGCCTAACTCAC |
| SEQ ID NO: 52 | TCCCACCCCAGCTAACTCACA |
| SEQ ID NO: 53 | CCCACCCCACGTAACTCACAC |
| SEQ ID NO: 54 | CCACCCCACCGAACTCACACA |
| SEQ ID NO: 55 | CACCCCACCTGACTCACACAA |
| SEQ ID NO: 56 | ACCCCACCTAGCTCACACAAA |
| SEQ ID NO: 57 | CCCCACCTAAGTCACACAAAC |
| SEQ ID NO: 58 | CCCACCTAACGCACACAAACC |
| SEQ ID NO: 59 | CCACCTAACTGACACAAACCA |
| SEQ ID NO: 60 | CACCTAACTCGCACAAACCAC |
| SEQ ID NO: 61 | ACCTAACTCAGACAAACCACC |
| SEQ ID NO: 62 | TACATTGCCCATGTAATTAA |
| SEQ ID NO: 63 | ATATAGTTTCGTCATTCATC |
| SEQ ID NO: 64 | TACATTGCCCATGTAATTAA |
| SEQ ID NO: 65 | ATATAGTTTCGTCATTCATC |
| SEQ ID NO: 66 | AGATAGTTTGGTCATTCATC |
| SEQ ID NO: 67 | AGATAGTTTCGTCATTCATC |
| SEQ ID NO: 68 | AGATAGTTTCGGCATTCATC |
| SEQ ID NO: 69 | AGATAGTTTCGTGATTCATC |
| SEQ ID NO: 70 | CCTAACTCACGCAAACCACCA |
| SEQ ID NO: 71 | CTAACTCACAGAAACCACCAA |
| SEQ ID NO: 72 | TAACTCACACGAACCACCAAC |
| SEQ ID NO: 73 | AACCAACCAACAATCTCCCAC |
| SEQ ID NO: 74 | ACCAACCAATCATCTCCCACC |
| SEQ ID NO: 75 | CCAACCAATACTCTCCCACCC |
| SEQ ID NO: 76 | CAACCAATAACCTCCCACCCC |
| SEQ ID NO: 77 | AACCAATAATCTCCCACCCCA |
| SEQ ID NO: 78 | ACCAATAATCCCCCACCCCAC |
| SEQ ID NO: 79 | CCAATAATCTCCCACCCCACC |
| SEQ ID NO: 80 | CAATAATCTCCCACCCCACCT |
| SEQ ID NO: 81 | AATAATCTCCCACCCCACCTA |
| SEQ ID NO: 82 | ATAATCTCCCCCCCCACCTAA |
| SEQ ID NO: 83 | TAATCTCCCACCCCACCTAAC |
| SEQ ID NO: 84 | AATCTCCCACCCCACCTAACT |
| SEQ ID NO: 85 | ATCTCCCACCCCACCTAACTC |
| SEQ ID NO: 86 | TCTCCCACCCCACCTAACTCA |
| SEQ ID NO: 87 | CTCCCACCCCCCCTAACTCAC |
| SEQ ID NO: 88 | TCCCACCCCACCTAACTCACA |
| SEQ ID NO: 89 | CCCACCCCACCTAACTCACAC |
| SEQ ID NO: 90 | CCACCCCACCCAACTCACACA |
| SEQ ID NO: 91 | CACCCCACCTCACTCACACAA |
| SEQ ID NO: 92 | ACCCCACCTACCTCACACAAA |
| SEQ ID NO: 93 | CCCCACCTAACTCACACAAAC |
| SEQ ID NO: 94 | CCCACCTAACCCACACAAACC |
| SEQ ID NO: 95 | CCACCTAACTCACACAAACCA |
| SEQ ID NO: 96 | CACCTAACTCCCACAAACCAC |
| SEQ ID NO: 97 | ACCTAACTCACACAAACCACC |
| SEQ ID NO: 98 | ATATAGTTTCGTCATTCATC |
| SEQ ID NO: 99 | TACATTGCCCATGTAATTAA |
| SEQ ID NO: 100 | ATATAGTTTCGTCATTCATC |
| SEQ ID NO: 101 | TACATTGCCCATGTAATTAA |
| SEQ ID NO: 102 | AGATAGTTTCGTCATTCATC |
| SEQ ID NO: 103 | AGATAGTTTCCTCATTCATC |
| SEQ ID NO: 104 | AGATAGTTTCGCCATTCATC |
| SEQ ID NO: 105 | AGATAGTTTCGTCATTCATC |
| SEQ ID NO: 106 | CCTAACTCACCCAAACCACCA |
| SEQ ID NO: 107 | CTAACTCACACAAACCACCAA |
| SEQ ID NO: 108 | TAACTCACACCAACCACCAAC |
| SEQ ID NO: 109 | AACCAACCAAAAATCTCCCAC |
| SEQ ID NO: 110 | ACCAACCAATAATCTCCCACC |
| SEQ ID NO: 111 | CCAACCAATAATCTCCCACCC |
| SEQ ID NO: 112 | CAACCAATAAACTCCCACCCC |
| SEQ ID NO: 113 | AACCAATAATATCCCACCCCA |
| SEQ ID NO: 114 | ACCAATAATCACCCACCCCAC |
| SEQ ID NO: 115 | CCAATAATCTACCACCCCACC |
| SEQ ID NO: 116 | CAATAATCTCACACCCCACCT |
| SEQ ID NO: 117 | AATAATCTCCAACCCCACCTA |
| SEQ ID NO: 118 | ATAATCTCCCACCCCACCTAA |
| SEQ ID NO: 119 | TAATCTCCCAACCCACCTAAC |
| SEQ ID NO: 120 | AATCTCCCACACCACCTAACT |
| SEQ ID NO: 121 | ATCTCCCACCACACCTAACTC |
| SEQ ID NO: 122 | TCTCCCACCCAACCTAACTCA |
| SEQ ID NO: 123 | CTCCCACCCCACCTAACTCAC |
| SEQ ID NO: 124 | TCCCACCCCAACTAACTCACA |
| SEQ ID NO: 125 | CCCACCCCACATAACTCACAC |
| SEQ ID NO: 126 | CCACCCCACCAAACTCACACA |
| SEQ ID NO: 127 | CACCCCACCTAACTCACACAA |
| SEQ ID NO: 128 | ACCCCACCTAACTCACACAAA |
| SEQ ID NO: 129 | CCCCACCTAAATCACACAAAC |
| SEQ ID NO: 130 | CCCACCTAACACACACAAACC |
| SEQ ID NO: 131 | CCACCTAACTAACACAAACCA |
| SEQ ID NO: 132 | CACCTAACTCACACAAACCAC |
| SEQ ID NO: 133 | ACCTAACTCAAACAAACCACC |
| SEQ ID NO: 134 | TACATTGCCCATGTAATTAA |
| SEQ ID NO: 135 | ATATAGTTTCGTCATTCATC |
| SEQ ID NO: 136 | TACATTGCCCATGTAATTAA |
| SEQ ID NO: 137 | ATATAGTTTCGTCATTCATC |
| SEQ ID NO: 138 | AGATAGTTTAGTCATTCATC |
| SEQ ID NO: 139 | AGATAGTTTCATCATTCATC |
| SEQ ID NO: 140 | AGATAGTTTCGACATTCATC |
| SEQ ID NO: 141 | AGATAGTTTCGTAATTCATC |
| SEQ ID NO: 142 | CCTAACTCACACAAACCACCA |
| SEQ ID NO: 143 | CTAACTCACAAAAACCACCAA |
| SEQ ID NO: 144 | TAACTCACACAAACCACCAAC |
| SEQ ID NO: 145 | AACTCACACATACCACCAACA |
| SEQ ID NO: 146 | ACTCACACAATCCACCAACAC |
| SEQ ID NO: 147 | CTCACACAAATCACCAACACC |
| SEQ ID NO: 148 | TCACACAAACTACCAACACCT |
| SEQ ID NO: 149 | CACACAAACCTCCAACACCTC |
| SEQ ID NO: 150 | ACACAAACCATCAACACCTCT |
| SEQ ID NO: 151 | CACAAACCACTAACACCTCTC |
| SEQ ID NO: 152 | ACAAACCACCTACACCTCTCC |
| SEQ ID NO: 153 | CAAACCACCATCACCTCTCCC |
| SEQ ID NO: 154 | AAACCACCAATACCTCTCCCC |
| SEQ ID NO: 155 | AACCACCAACTCCTCTCCCCC |
| SEQ ID NO: 156 | ACCACCAACATCTCTCCCCCT |
| SEQ ID NO: 157 | CCACCAACACTTCTCCCCCTC |
| SEQ ID NO: 158 | CACCAACACCTCTCCCCCTCT |
| SEQ ID NO: 159 | ACCAACACCTTTCCCCCTCTC |
| SEQ ID NO: 160 | CCAACACCTCTCCCCCTCTCA |
| SEQ ID NO: 161 | CAACACCTCTTCCCCTCTCAT |
| SEQ ID NO: 162 | AACACCTCTCTCCCTCTCATC |
| SEQ ID NO: 163 | ACACCTCTCCTCCTCTCATCC |
| SEQ ID NO: 164 | CACCTCTCCCTCTCTCATCCA |
| SEQ ID NO: 165 | ACCTCTCCCCTTCTCATCCAT |
| SEQ ID NO: 166 | CCTCTCCCCCTCTCATCCATC |
| SEQ ID NO: 167 | ATATAGTTTCGTCATTCATC |
| SEQ ID NO: 168 | TACATTGCCCATGTAATTAA |
| SEQ ID NO: 169 | ATATAGTTTCGTCATTCATC |
| SEQ ID NO: 170 | TACATTGCCCATGTAATTAA |
| SEQ ID NO: 171 | AGATAGTTTTGTCATTCATC |
| SEQ ID NO: 172 | AGATAGTTTCTTCATTCATC |
| SEQ ID NO: 173 | AGATAGTTTCGTCATTCATC |
| SEQ ID NO: 174 | AGATAGTTTCGTTATTCATC |
| SEQ ID NO: 175 | CTCTCCCCCTTTCATCCATCA |
| SEQ ID NO: 176 | TCTCCCCCTCTCATCCATCAC |
| SEQ ID NO: 177 | CTCCCCCTCTTATCCATCACC |
| SEQ ID NO: 178 | TCCCCCTCTCTTCCATCACCC |
| SEQ ID NO: 179 | CCCCCTCTCATCCATCACCCA |
| SEQ ID NO: 180 | CCCCTCTCATTCATCACCCAC |
| SEQ ID NO: 181 | AACTCACACAGACCACCAACA |
| SEQ ID NO: 182 | ACTCACACAAGCCACCAACAC |
| SEQ ID NO: 183 | CTCACACAAAGCACCAACACC |
| SEQ ID NO: 184 | TCACACAAACGACCAACACCT |
| SEQ ID NO: 185 | CACACAAACCGCCAACACCTC |
| SEQ ID NO: 186 | ACACAAACCAGCAACACCTCT |
| SEQ ID NO: 187 | CACAAACCACGAACACCTCTC |
| SEQ ID NO: 188 | ACAAACCACCGACACCTCTCC |
| SEQ ID NO: 189 | CAAACCACCAGCACCTCTCCC |
| SEQ ID NO: 190 | AAACCACCAAGACCTCTCCCC |
| SEQ ID NO: 191 | AACCACCAACGCCTCTCCCCC |
| SEQ ID NO: 192 | ACCACCAACAGCTCTCCCCCT |
| SEQ ID NO: 193 | CCACCAACACGTCTCCCCCTC |
| SEQ ID NO: 194 | CACCAACACCGCTCCCCCTCT |
| SEQ ID NO: 195 | ACCAACACCTGTCCCCCTCTC |
| SEQ ID NO: 196 | CCAACACCTCGCCCCCTCTCA |
| SEQ ID NO: 197 | CAACACCTCTGCCCCTCTCAT |
| SEQ ID NO: 198 | AACACCTCTCGCCCTCTCATC |
| SEQ ID NO: 199 | ACACCTCTCCGCCTCTCATCC |
| SEQ ID NO: 200 | CACCTCTCCCGCTCTCATCCA |
| SEQ ID NO: 201 | ACCTCTCCCCGTCTCATCCAT |
| SEQ ID NO: 202 | CCTCTCCCCCGCTCATCCATC |
| SEQ ID NO: 203 | TACATTGCCCATGTAATTAA |
| SEQ ID NO: 204 | ATATAGTTTCGTCATTCATC |
| SEQ ID NO: 205 | TACATTGCCCATGTAATTAA |
| SEQ ID NO: 206 | ATATAGTTTCGTCATTCATC |
| SEQ ID NO: 207 | AGATAGTTTGGTCATTCATC |
| SEQ ID NO: 208 | AGATAGTTTCGTCATTCATC |
| SEQ ID NO: 209 | AGATAGTTTCGGCATTCATC |
| SEQ ID NO: 210 | AGATAGTTTCGTGATTCATC |
| SEQ ID NO: 211 | CTCTCCCCCTGTCATCCATCA |
| SEQ ID NO: 212 | TCTCCCCCTCGCATCCATCAC |
| SEQ ID NO: 213 | CTCCCCCTCTGATCCATCACC |
| SEQ ID NO: 214 | TCCCCCTCTCGTCCATCACCC |
| SEQ ID NO: 215 | CCCCCTCTCAGCCATCACCCA |
| SEQ ID NO: 216 | CCCCTCTCATGCATCACCCAC |
| SEQ ID NO: 217 | AACTCACACACACCACCAACA |
| SEQ ID NO: 218 | ACTCACACAACCCACCAACAC |
| SEQ ID NO: 219 | CTCACACAAACCACCAACACC |
| SEQ ID NO: 220 | TCACACAAACCACCAACACCT |
| SEQ ID NO: 221 | CACACAAACCCCCAACACCTC |
| SEQ ID NO: 222 | ACACAAACCACCAACACCTCT |
| SEQ ID NO: 223 | CACAAACCACCAACACCTCTC |
| SEQ ID NO: 224 | ACAAACCACCCACACCTCTCC |
| SEQ ID NO: 225 | CAAACCACCACCACCTCTCCC |
| SEQ ID NO: 226 | AAACCACCAACACCTCTCCCC |
| SEQ ID NO: 227 | AACCACCAACCCCTCTCCCCC |
| SEQ ID NO: 228 | ACCACCAACACCTCTCCCCCT |
| SEQ ID NO: 229 | CCACCAACACCTCTCCCCCTC |
| SEQ ID NO: 230 | CACCAACACCCCTCCCCCTCT |
| SEQ ID NO: 231 | ACCAACACCTCTCCCCCTCTC |
| SEQ ID NO: 232 | CCAACACCTCCCCCCCTCTCA |
| SEQ ID NO: 233 | CAACACCTCTCCCCCTCTCAT |
| SEQ ID NO: 234 | AACACCTCTCCCCCTCTCATC |
| SEQ ID NO: 235 | ACACCTCTCCCCCTCTCATCC |
| SEQ ID NO: 236 | CACCTCTCCCCCTCTCATCCA |
| SEQ ID NO: 237 | ACCTCTCCCCCTCTCATCCAT |
| SEQ ID NO: 238 | CCTCTCCCCCCCTCATCCATC |
| SEQ ID NO: 239 | ATATAGTTTCGTCATTCATC |
| SEQ ID NO: 240 | TACATTGCCCATGTAATTAA |
| SEQ ID NO: 241 | ATATAGTTTCGTCATTCATC |
| SEQ ID NO: 242 | TACATTGCCCATGTAATTAA |
| SEQ ID NO: 243 | AGATAGTTTCGTCATTCATC |
| SEQ ID NO: 244 | AGATAGTTTCCTCATTCATC |
| SEQ ID NO: 245 | AGATAGTTTCGCCATTCATC |
| SEQ ID NO: 246 | AGATAGTTTCGTCATTCATC |
| SEQ ID NO: 247 | CTCTCCCCCTCTCATCCATCA |
| SEQ ID NO: 248 | TCTCCCCCTCCCATCCATCAC |
| SEQ ID NO: 249 | CTCCCCCTCTCATCCATCACC |
| SEQ ID NO: 250 | TCCCCCTCTCCTCCATCACCC |
| SEQ ID NO: 251 | CCCCCTCTCACCCATCACCCA |
| SEQ ID NO: 252 | CCCCTCTCATCCATCACCCAC |
| SEQ ID NO: 253 | AACTCACACAAACCACCAACA |
| SEQ ID NO: 254 | ACTCACACAAACCACCAACAC |
| SEQ ID NO: 255 | CTCACACAAAACACCAACACC |
| SEQ ID NO: 256 | TCACACAAACAACCAACACCT |
| SEQ ID NO: 257 | CACACAAACCACCAACACCTC |
| SEQ ID NO: 258 | ACACAAACCAACAACACCTCT |
| SEQ ID NO: 259 | CACAAACCACAAACACCTCTC |
| SEQ ID NO: 260 | ACAAACCACCAACACCTCTCC |
| SEQ ID NO: 261 | CAAACCACCAACACCTCTCCC |
| SEQ ID NO: 262 | AAACCACCAAAACCTCTCCCC |
| SEQ ID NO: 263 | AACCACCAACACCTCTCCCCC |
| SEQ ID NO: 264 | ACCACCAACAACTCTCCCCCT |
| SEQ ID NO: 265 | CCACCAACACATCTCCCCCTC |
| SEQ ID NO: 266 | CACCAACACCACTCCCCCTCT |
| SEQ ID NO: 267 | ACCAACACCTATCCCCCTCTC |
| SEQ ID NO: 268 | CCAACACCTCACCCCCTCTCA |
| SEQ ID NO: 269 | CAACACCTCTACCCCTCTCAT |
| SEQ ID NO: 270 | AACACCTCTCACCCTCTCATC |
| SEQ ID NO: 271 | ACACCTCTCCACCTCTCATCC |
| SEQ ID NO: 272 | CACCTCTCCCACTCTCATCCA |
| SEQ ID NO: 273 | ACCTCTCCCCATCTCATCCAT |
| SEQ ID NO: 274 | CCTCTCCCCCACTCATCCATC |
| SEQ ID NO: 275 | TACATTGCCCATGTAATTAA |
| SEQ ID NO: 276 | ATATAGTTTCGTCATTCATC |
| SEQ ID NO: 277 | TACATTGCCCATGTAATTAA |
| SEQ ID NO: 278 | ATATAGTTTCGTCATTCATC |
| SEQ ID NO: 279 | AGATAGTTTAGTCATTCATC |
| SEQ ID NO: 280 | AGATAGTTTCATCATTCATC |
| SEQ ID NO: 281 | AGATAGTTTCGACATTCATC |
| SEQ ID NO: 282 | AGATAGTTTCGTAATTCATC |
| SEQ ID NO: 283 | CTCTCCCCCTATCATCCATCA |
| SEQ ID NO: 284 | TCTCCCCCTCACATCCATCAC |
| SEQ ID NO: 285 | CTCCCCCTCTAATCCATCACC |
| SEQ ID NO: 286 | TCCCCCTCTCATCCATCACCC |
| SEQ ID NO: 287 | CCCCCTCTCAACCATCACCCA |
| SEQ ID NO: 288 | CCCCTCTCATACATCACCCAC |
| SEQ ID NO: 289 | CCCTCTCATCTATCACCCACC |
| SEQ ID NO: 290 | CCTCTCATCCTTCACCCACCA |
| SEQ ID NO: 291 | CTCTCATCCATCACCCACCAC |
| SEQ ID NO: 292 | TCTCATCCATTACCCACCACC |
| SEQ ID NO: 293 | CTCATCCATCTCCCACCACCC |
| SEQ ID NO: 294 | TCATCCATCATCCACCACCCC |
| SEQ ID NO: 295 | CATCCATCACTCACCACCCCT |
| SEQ ID NO: 296 | ATCCATCACCTACCACCCCTC |
| SEQ ID NO: 297 | TCCATCACCCTCCACCCCTCA |
| SEQ ID NO: 298 | CCATCACCCATCACCCCTCAT |
| SEQ ID NO: 299 | CATCACCCACTACCCCTCATC |
| SEQ ID NO: 300 | ATCACCCACCTCCCCTCATCA |
| SEQ ID NO: 301 | TCACCCACCATCCCTCATCAT |
| SEQ ID NO: 302 | CACCCACCACTCCTCATCATA |
| SEQ ID NO: 303 | ACCCACCACCTCTCATCATAC |
| SEQ ID NO: 304 | CCCACCACCCTTCATCATACC |
| SEQ ID NO: 305 | CCACCACCCCTCATCATACCT |
| SEQ ID NO: 306 | CACCACCCCTTATCATACCTC |
| SEQ ID NO: 307 | ACCACCCCTCTTCATACCTCA |
| SEQ ID NO: 308 | ATATAGTTTCGTCATTCATC |
| SEQ ID NO: 309 | TACATTGCCCATGTAATTAA |
| SEQ ID NO: 310 | ATATAGTTTCGTCATTCATC |
| SEQ ID NO: 311 | TACATTGCCCATGTAATTAA |
| SEQ ID NO: 312 | AGATAGTTTTGTCATTCATC |
| SEQ ID NO: 313 | AGATAGTTTCTTCATTCATC |
| SEQ ID NO: 314 | AGATAGTTTCGTCATTCATC |
| SEQ ID NO: 315 | AGATAGTTTCGTTATTCATC |
| SEQ ID NO: 316 | CCACCCCTCATCATACCTCAA |
| SEQ ID NO: 317 | CACCCCTCATTATACCTCAAC |
| SEQ ID NO: 318 | ACCCCTCATCTTACCTCAACC |
| SEQ ID NO: 319 | CCCCTCATCATACCTCAACCA |
| SEQ ID NO: 320 | CCCTCATCATTCCTCAACCAC |
| SEQ ID NO: 321 | CCTCATCATATCTCAACCACC |
| SEQ ID NO: 322 | CTCATCATACTTCAACCACCA |
| SEQ ID NO: 323 | TCATCATACCTCAACCACCAC |
| SEQ ID NO: 324 | CATCATACCTTAACCACCACC |
| SEQ ID NO: 325 | CCCTCTCATCGATCACCCACC |
| SEQ ID NO: 326 | CCTCTCATCCGTCACCCACCA |
| SEQ ID NO: 327 | CTCTCATCCAGCACCCACCAC |
| SEQ ID NO: 328 | TCTCATCCATGACCCACCACC |
| SEQ ID NO: 329 | CTCATCCATCGCCCACCACCC |
| SEQ ID NO: 330 | TCATCCATCAGCCACCACCCC |
| SEQ ID NO: 331 | CATCCATCACGCACCACCCCT |
| SEQ ID NO: 332 | ATCCATCACCGACCACCCCTC |
| SEQ ID NO: 333 | TCCATCACCCGCCACCCCTCA |
| SEQ ID NO: 334 | CCATCACCCAGCACCCCTCAT |
| SEQ ID NO: 335 | CATCACCCACGACCCCTCATC |
| SEQ ID NO: 336 | ATCACCCACCGCCCCTCATCA |
| SEQ ID NO: 337 | TCACCCACCAGCCCTCATCAT |
| SEQ ID NO: 338 | CACCCACCACGCCTCATCATA |
| SEQ ID NO: 339 | ACCCACCACCGCTCATCATAC |
| SEQ ID NO: 340 | CCCACCACCCGTCATCATACC |
| SEQ ID NO: 341 | CCACCACCCCGCATCATACCT |
| SEQ ID NO: 342 | CACCACCCCTGATCATACCTC |
| SEQ ID NO: 343 | ACCACCCCTCGTCATACCTCA |
| SEQ ID NO: 344 | TACATTGCCCATGTAATTAA |
| SEQ ID NO: 345 | ATATAGTTTCGTCATTCATC |
| SEQ ID NO: 346 | TACATTGCCCATGTAATTAA |
| SEQ ID NO: 347 | ATATAGTTTCGTCATTCATC |
| SEQ ID NO: 348 | AGATAGTTTGGTCATTCATC |
| SEQ ID NO: 349 | AGATAGTTTCGTCATTCATC |
| SEQ ID NO: 350 | AGATAGTTTCGGCATTCATC |
| SEQ ID NO: 351 | AGATAGTTTCGTGATTCATC |
| SEQ ID NO: 352 | CCACCCCTCAGCATACCTCAA |
| SEQ ID NO: 353 | CACCCCTCATGATACCTCAAC |
| SEQ ID NO: 354 | ACCCCTCATCGTACCTCAACC |
| SEQ ID NO: 355 | CCCCTCATCAGACCTCAACCA |
| SEQ ID NO: 356 | CCCTCATCATGCCTCAACCAC |
| SEQ ID NO: 357 | CCTCATCATAGCTCAACCACC |
| SEQ ID NO: 358 | CTCATCATACGTCAACCACCA |
| SEQ ID NO: 359 | TCATCATACCGCAACCACCAC |
| SEQ ID NO: 360 | CATCATACCTGAACCACCACC |
| SEQ ID NO: 361 | CCCTCTCATCCATCACCCACC |
| SEQ ID NO: 362 | CCTCTCATCCCTCACCCACCA |
| SEQ ID NO: 363 | CTCTCATCCACCACCCACCAC |
| SEQ ID NO: 364 | TCTCATCCATCACCCACCACC |
| SEQ ID NO: 365 | CTCATCCATCCCCCACCACCC |
| SEQ ID NO: 366 | TCATCCATCACCCACCACCCC |
| SEQ ID NO: 367 | CATCCATCACCCACCACCCCT |
| SEQ ID NO: 368 | ATCCATCACCCACCACCCCTC |
| SEQ ID NO: 369 | TCCATCACCCCCCACCCCTCA |
| SEQ ID NO: 370 | CCATCACCCACCACCCCTCAT |
| SEQ ID NO: 371 | CATCACCCACCACCCCTCATC |
| SEQ ID NO: 372 | ATCACCCACCCCCCCTCATCA |
| SEQ ID NO: 373 | TCACCCACCACCCCTCATCAT |
| SEQ ID NO: 374 | CACCCACCACCCCTCATCATA |
| SEQ ID NO: 375 | ACCCACCACCCCTCATCATAC |
| SEQ ID NO: 376 | CCCACCACCCCTCATCATACC |
| SEQ ID NO: 377 | CCACCACCCCCCATCATACCT |
| SEQ ID NO: 378 | CACCACCCCTCATCATACCTC |
| SEQ ID NO: 379 | ACCACCCCTCCTCATACCTCA |
| SEQ ID NO: 380 | ATATAGTTTCGTCATTCATC |
| SEQ ID NO: 381 | TACATTGCCCATGTAATTAA |
| SEQ ID NO: 382 | ATATAGTTTCGTCATTCATC |
| SEQ ID NO: 383 | TACATTGCCCATGTAATTAA |
| SEQ ID NO: 384 | AGATAGTTTCGTCATTCATC |
| SEQ ID NO: 385 | AGATAGTTTCCTCATTCATC |
| SEQ ID NO: 386 | AGATAGTTTCGCCATTCATC |
| SEQ ID NO: 387 | AGATAGTTTCGTCATTCATC |
| SEQ ID NO: 388 | CCACCCCTCACCATACCTCAA |
| SEQ ID NO: 389 | CACCCCTCATCATACCTCAAC |
| SEQ ID NO: 390 | ACCCCTCATCCTACCTCAACC |
| SEQ ID NO: 391 | CCCCTCATCACACCTCAACCA |
| SEQ ID NO: 392 | CCCTCATCATCCCTCAACCAC |
| SEQ ID NO: 393 | CCTCATCATACCTCAACCACC |
| SEQ ID NO: 394 | CTCATCATACCTCAACCACCA |
| SEQ ID NO: 395 | TCATCATACCCCAACCACCAC |
| SEQ ID NO: 396 | CATCATACCTCAACCACCACC |
| SEQ ID NO: 397 | CCCTCTCATCAATCACCCACC |
| SEQ ID NO: 398 | CCTCTCATCCATCACCCACCA |
| SEQ ID NO: 399 | CTCTCATCCAACACCCACCAC |
| SEQ ID NO: 400 | TCTCATCCATAACCCACCACC |
| SEQ ID NO: 401 | CTCATCCATCACCCACCACCC |
| SEQ ID NO: 402 | TCATCCATCAACCACCACCCC |
| SEQ ID NO: 403 | CATCCATCACACACCACCCCT |
| SEQ ID NO: 404 | ATCCATCACCAACCACCCCTC |
| SEQ ID NO: 405 | TCCATCACCCACCACCCCTCA |
| SEQ ID NO: 406 | CCATCACCCAACACCCCTCAT |
| SEQ ID NO: 407 | CATCACCCACAACCCCTCATC |
| SEQ ID NO: 408 | ATCACCCACCACCCCTCATCA |
| SEQ ID NO: 409 | TCACCCACCAACCCTCATCAT |
| SEQ ID NO: 410 | CACCCACCACACCTCATCATA |
| SEQ ID NO: 411 | ACCCACCACCACTCATCATAC |
| SEQ ID NO: 412 | CCCACCACCCATCATCATACC |
| SEQ ID NO: 413 | CCACCACCCCACATCATACCT |
| SEQ ID NO: 414 | CACCACCCCTAATCATACCTC |
| SEQ ID NO: 415 | ACCACCCCTCATCATACCTCA |
| SEQ ID NO: 416 | TACATTGCCCATGTAATTAA |
| SEQ ID NO: 417 | ATATAGTTTCGTCATTCATC |
| SEQ ID NO: 418 | TACATTGCCCATGTAATTAA |
| SEQ ID NO: 419 | ATATAGTTTCGTCATTCATC |
| SEQ ID NO: 420 | AGATAGTTTAGTCATTCATC |
| SEQ ID NO: 421 | AGATAGTTTCATCATTCATC |
| SEQ ID NO: 422 | AGATAGTTTCGACATTCATC |
| SEQ ID NO: 423 | AGATAGTTTCGTAATTCATC |
| SEQ ID NO: 424 | CCACCCCTCAACATACCTCAA |
| SEQ ID NO: 425 | CACCCCTCATAATACCTCAAC |
| SEQ ID NO: 426 | ACCCCTCATCATACCTCAACC |
| SEQ ID NO: 427 | CCCCTCATCAAACCTCAACCA |
| SEQ ID NO: 428 | CCCTCATCATACCTCAACCAC |
| SEQ ID NO: 429 | CCTCATCATAACTCAACCACC |
| SEQ ID NO: 430 | CTCATCATACATCAACCACCA |
| SEQ ID NO: 431 | TCATCATACCACAACCACCAC |
| SEQ ID NO: 432 | CATCATACCTAAACCACCACC |
| SEQ ID NO: 433 | ATCATACCTCTACCACCACCC |
| SEQ ID NO: 434 | TCATACCTCATCCACCACCCC |
| SEQ ID NO: 435 | CATACCTCAATCACCACCCCT |
| SEQ ID NO: 436 | ATACCTCAACTACCACCCCTC |
| SEQ ID NO: 437 | TACCTCAACCTCCACCCCTCA |
| SEQ ID NO: 438 | ACCTCAACCATCACCCCTCAT |
| SEQ ID NO: 439 | CCTCAACCACTACCCCTCATC |
| SEQ ID NO: 440 | CTCAACCACCTCCCCTCATCA |
| SEQ ID NO: 441 | TCAACCACCATCCCTCATCAT |
| SEQ ID NO: 442 | CAACCACCACTCCTCATCATA |
| SEQ ID NO: 443 | AACCACCACCTCTCATCATAC |
| SEQ ID NO: 444 | ACCACCACCCTTCATCATACC |
| SEQ ID NO: 445 | CCACCACCCCTCATCATACCT |
| SEQ ID NO: 446 | CACCACCCCTTATCATACCTC |
| SEQ ID NO: 447 | ACCACCCCTCTTCATACCTCA |
| SEQ ID NO: 448 | CCACCCCTCATCATACCTCAA |
| SEQ ID NO: 449 | ATATAGTTTCGTCATTCATC |
| SEQ ID NO: 450 | TACATTGCCCATGTAATTAA |
| SEQ ID NO: 451 | ATATAGTTTCGTCATTCATC |
| SEQ ID NO: 452 | TACATTGCCCATGTAATTAA |
| SEQ ID NO: 453 | AGATAGTTTTGTCATTCATC |
| SEQ ID NO: 454 | AGATAGTTTCTTCATTCATC |
| SEQ ID NO: 455 | AGATAGTTTCGTCATTCATC |
| SEQ ID NO: 456 | AGATAGTTTCGTTATTCATC |
| SEQ ID NO: 457 | CACCCCTCATTATACCTCAAA |
| SEQ ID NO: 458 | ACCCCTCATCTTACCTCAAAA |
| SEQ ID NO: 459 | CCCCTCATCATACCTCAAAAA |
| SEQ ID NO: 460 | CCCTCATCATTCCTCAAAAAC |
| SEQ ID NO: 461 | CCTCATCATATCTCAAAAACC |
| SEQ ID NO: 462 | CTCATCATACTTCAAAAACCA |
| SEQ ID NO: 463 | TCATCATACCTCAAAAACCAA |
| SEQ ID NO: 464 | CATCATACCTTAAAAACCAAC |
| SEQ ID NO: 465 | ATCATACCTCTAAAACCAACT |
| SEQ ID NO: 466 | TCATACCTCATAAACCAACTA |
| SEQ ID NO: 467 | CATACCTCAATAACCAACTAA |
| SEQ ID NO: 468 | ATACCTCAAATACCAACTAAC |
| SEQ ID NO: 469 | ATCATACCTCGACCACCACCC |
| SEQ ID NO: 470 | TCATACCTCAGCCACCACCCC |
| SEQ ID NO: 471 | CATACCTCAAGCACCACCCCT |
| SEQ ID NO: 472 | ATACCTCAACGACCACCCCTC |
| SEQ ID NO: 473 | TACCTCAACCGCCACCCCTCA |
| SEQ ID NO: 474 | ACCTCAACCAGCACCCCTCAT |
| SEQ ID NO: 475 | CCTCAACCACGACCCCTCATC |
| SEQ ID NO: 476 | CTCAACCACCGCCCCTCATCA |
| SEQ ID NO: 477 | TCAACCACCAGCCCTCATCAT |
| SEQ ID NO: 478 | CAACCACCACGCCTCATCATA |
| SEQ ID NO: 479 | AACCACCACCGCTCATCATAC |
| SEQ ID NO: 480 | ACCACCACCCGTCATCATACC |
| SEQ ID NO: 481 | CCACCACCCCGCATCATACCT |
| SEQ ID NO: 482 | CACCACCCCTGATCATACCTC |
| SEQ ID NO: 483 | ACCACCCCTCGTCATACCTCA |
| SEQ ID NO: 484 | CCACCCCTCAGCATACCTCAA |
| SEQ ID NO: 485 | TACATTGCCCATGTAATTAA |
| SEQ ID NO: 486 | ATATAGTTTCGTCATTCATC |
| SEQ ID NO: 487 | TACATTGCCCATGTAATTAA |
| SEQ ID NO: 488 | ATATAGTTTCGTCATTCATC |
| SEQ ID NO: 489 | AGATAGTTTGGTCATTCATC |
| SEQ ID NO: 490 | AGATAGTTTCGTCATTCATC |
| SEQ ID NO: 491 | AGATAGTTTCGGCATTCATC |
| SEQ ID NO: 492 | AGATAGTTTCGTGATTCATC |
| SEQ ID NO: 493 | CACCCCTCATGATACCTCAAA |
| SEQ ID NO: 494 | ACCCCTCATCGTACCTCAAAA |
| SEQ ID NO: 495 | CCCCTCATCAGACCTCAAAAA |
| SEQ ID NO: 496 | CCCTCATCATGCCTCAAAAAC |
| SEQ ID NO: 497 | CCTCATCATAGCTCAAAAACC |
| SEQ ID NO: 498 | CTCATCATACGTCAAAAACCA |
| SEQ ID NO: 499 | TCATCATACCGCAAAAACCAA |
| SEQ ID NO: 500 | CATCATACCTGAAAAACCAAC |
| SEQ ID NO: 501 | ATCATACCTCGAAAACCAACT |
| SEQ ID NO: 502 | TCATACCTCAGAAACCAACTA |
| SEQ ID NO: 503 | CATACCTCAAGAACCAACTAA |
| SEQ ID NO: 504 | ATACCTCAAAGACCAACTAAC |
| SEQ ID NO: 505 | ATCATACCTCCACCACCACCC |
| SEQ ID NO: 506 | TCATACCTCACCCACCACCCC |
| SEQ ID NO: 507 | CATACCTCAACCACCACCCCT |
| SEQ ID NO: 508 | ATACCTCAACCACCACCCCTC |
| SEQ ID NO: 509 | TACCTCAACCCCCACCCCTCA |
| SEQ ID NO: 510 | ACCTCAACCACCACCCCTCAT |
| SEQ ID NO: 511 | CCTCAACCACCACCCCTCATC |
| SEQ ID NO: 512 | CTCAACCACCCCCCCTCATCA |
| SEQ ID NO: 513 | TCAACCACCACCCCTCATCAT |
| SEQ ID NO: 514 | CAACCACCACCCCTCATCATA |
| SEQ ID NO: 515 | AACCACCACCCCTCATCATAC |
| SEQ ID NO: 516 | ACCACCACCCCTCATCATACC |
| SEQ ID NO: 517 | CCACCACCCCCCATCATACCT |
| SEQ ID NO: 518 | CACCACCCCTCATCATACCTC |
| SEQ ID NO: 519 | ACCACCCCTCCTCATACCTCA |
| SEQ ID NO: 520 | CCACCCCTCACCATACCTCAA |
| SEQ ID NO: 521 | ATATAGTTTCGTCATTCATC |
| SEQ ID NO: 522 | TACATTGCCCATGTAATTAA |
| SEQ ID NO: 523 | ATATAGTTTCGTCATTCATC |
| SEQ ID NO: 524 | TACATTGCCCATGTAATTAA |
| SEQ ID NO: 525 | AGATAGTTTCGTCATTCATC |
| SEQ ID NO: 526 | AGATAGTTTCCTCATTCATC |
| SEQ ID NO: 527 | AGATAGTTTCGCCATTCATC |
| SEQ ID NO: 528 | AGATAGTTTCGTCATTCATC |
| SEQ ID NO: 529 | CACCCCTCATCATACCTCAAA |
| SEQ ID NO: 530 | ACCCCTCATCCTACCTCAAAA |
| SEQ ID NO: 531 | CCCCTCATCACACCTCAAAAA |
| SEQ ID NO: 532 | CCCTCATCATCCCTCAAAAAC |
| SEQ ID NO: 533 | CCTCATCATACCTCAAAAACC |
| SEQ ID NO: 534 | CTCATCATACCTCAAAAACCA |
| SEQ ID NO: 535 | TCATCATACCCCAAAAACCAA |
| SEQ ID NO: 536 | CATCATACCTCAAAAACCAAC |
| SEQ ID NO: 537 | ATCATACCTCCAAAACCAACT |
| SEQ ID NO: 538 | TCATACCTCACAAACCAACTA |
| SEQ ID NO: 539 | CATACCTCAACAACCAACTAA |
| SEQ ID NO: 540 | ATACCTCAAACACCAACTAAC |
| SEQ ID NO: 541 | ATCATACCTCAACCACCACCC |
| SEQ ID NO: 542 | TCATACCTCAACCACCACCCC |
| SEQ ID NO: 543 | CATACCTCAAACACCACCCCT |
| SEQ ID NO: 544 | ATACCTCAACAACCACCCCTC |
| SEQ ID NO: 545 | TACCTCAACCACCACCCCTCA |
| SEQ ID NO: 546 | ACCTCAACCAACACCCCTCAT |
| SEQ ID NO: 547 | CCTCAACCACAACCCCTCATC |
| SEQ ID NO: 548 | CTCAACCACCACCCCTCATCA |
| SEQ ID NO: 549 | TCAACCACCAACCCTCATCAT |
| SEQ ID NO: 550 | CAACCACCACACCTCATCATA |
| SEQ ID NO: 551 | AACCACCACCACTCATCATAC |
| SEQ ID NO: 552 | ACCACCACCCATCATCATACC |
| SEQ ID NO: 553 | CCACCACCCCACATCATACCT |
| SEQ ID NO: 554 | CACCACCCCTAATCATACCTC |
| SEQ ID NO: 555 | ACCACCCCTCATCATACCTCA |
| SEQ ID NO: 556 | CCACCCCTCAACATACCTCAA |
| SEQ ID NO: 557 | TACATTGCCCATGTAATTAA |
| SEQ ID NO: 558 | ATATAGTTTCGTCATTCATC |
| SEQ ID NO: 559 | TACATTGCCCATGTAATTAA |
| SEQ ID NO: 560 | ATATAGTTTCGTCATTCATC |
| SEQ ID NO: 561 | AGATAGTTTAGTCATTCATC |
| SEQ ID NO: 562 | AGATAGTTTCATCATTCATC |
| SEQ ID NO: 563 | AGATAGTTTCGACATTCATC |
| SEQ ID NO: 564 | AGATAGTTTCGTAATTCATC |
| SEQ ID NO: 565 | CACCCCTCATAATACCTCAAA |
| SEQ ID NO: 566 | ACCCCTCATCATACCTCAAAA |
| SEQ ID NO: 567 | CCCCTCATCAAACCTCAAAAA |
| SEQ ID NO: 568 | CCCTCATCATACCTCAAAAAC |
| SEQ ID NO: 569 | CCTCATCATAACTCAAAAACC |
| SEQ ID NO: 570 | CTCATCATACATCAAAAACCA |
| SEQ ID NO: 571 | TCATCATACCACAAAAACCAA |
| SEQ ID NO: 572 | CATCATACCTAAAAAACCAAC |
| SEQ ID NO: 573 | ATCATACCTCAAAAACCAACT |
| SEQ ID NO: 574 | TCATACCTCAAAAACCAACTA |
| SEQ ID NO: 575 | CATACCTCAAAAACCAACTAA |
| SEQ ID NO: 576 | ATACCTCAAAAACCAACTAAC |
| SEQ ID NO: 577 | TACCTCAAAATCCAACTAACC |
| SEQ ID NO: 578 | ACCTCAAAAATCAACTAACCA |
| SEQ ID NO: 579 | CCTCAAAAACTAACTAACCAA |
| SEQ ID NO: 580 | CTCAAAAACCTACTAACCAAC |
| SEQ ID NO: 581 | TCAAAAACCATCTAACCAACC |
| SEQ ID NO: 582 | CAAAAACCAATTAACCAACCA |
| SEQ ID NO: 583 | AAAAACCAACTAACCAACCAA |
| SEQ ID NO: 584 | AAAACCAACTTACCAACCAAT |
| SEQ ID NO: 585 | AACCAACCAATAATCTCCCAC |
| SEQ ID NO: 586 | ACCAACCAATTATCTCCCACC |
| SEQ ID NO: 587 | CCAACCAATATTCTCCCACCC |
| SEQ ID NO: 588 | CAACCAATAATCTCCCACCCC |
| SEQ ID NO: 589 | AACCAATAATTTCCCACCCCG |
| SEQ ID NO: 590 | ATATAGTTTCGTCATTCATC |
| SEQ ID NO: 591 | TACATTGCCCATGTAATTAA |
| SEQ ID NO: 592 | ATATAGTTTCGTCATTCATC |
| SEQ ID NO: 593 | TACATTGCCCATGTAATTAA |
| SEQ ID NO: 594 | AGATAGTTTTGTCATTCATC |
| SEQ ID NO: 595 | AGATAGTTTCTTCATTCATC |
| SEQ ID NO: 596 | AGATAGTTTCGTCATTCATC |
| SEQ ID NO: 597 | AGATAGTTTCGTTATTCATC |
| SEQ ID NO: 598 | ACCAATAATCTCCCACCCCGC |
| SEQ ID NO: 599 | CCAATAATCTTCCACCCCGCC |
| SEQ ID NO: 600 | CAATAATCTCTCACCCCGCCT |
| SEQ ID NO: 601 | AATAATCTCCTACCCCGCCTA |
| SEQ ID NO: 602 | ATAATCTCCCTCCCCGCCTAG |
| SEQ ID NO: 603 | TAATCTCCCATCCCGCCTAGC |
| SEQ ID NO: 604 | AATCTCCCACTCCGCCTAGCT |
| SEQ ID NO: 605 | ATCTCCCACCTCGCCTAGCTC |
| SEQ ID NO: 606 | TCTCCCACCCTGCCTAGCTCA |
| SEQ ID NO: 607 | CTCCCACCCCTCCTAGCTCAC |
| SEQ ID NO: 608 | TCCCACCCCGTCTAGCTCACG |
| SEQ ID NO: 609 | CCCACCCCGCTTAGCTCACGC |
| SEQ ID NO: 610 | CCACCCCGCCTAGCTCACGCA |
| SEQ ID NO: 611 | CACCCCGCCTTGCTCACGCAA |
| SEQ ID NO: 612 | ACCCCGCCTATCTCACGCAAG |
| SEQ ID NO: 613 | TACCTCAAAAGCCAACTAACC |
| SEQ ID NO: 614 | ACCTCAAAAAGCAACTAACCA |
| SEQ ID NO: 615 | CCTCAAAAACGAACTAACCAA |
| SEQ ID NO: 616 | CTCAAAAACCGACTAACCAAC |
| SEQ ID NO: 617 | TCAAAAACCAGCTAACCAACC |
| SEQ ID NO: 618 | CAAAAACCAAGTAACCAACCA |
| SEQ ID NO: 619 | AAAAACCAACGAACCAACCAA |
| SEQ ID NO: 620 | AAAACCAACTGACCAACCAAT |
| SEQ ID NO: 621 | AACCAACCAAGAATCTCCCAC |
| SEQ ID NO: 622 | ACCAACCAATGATCTCCCACC |
| SEQ ID NO: 623 | CCAACCAATAGTCTCCCACCC |
| SEQ ID NO: 624 | CAACCAATAAGCTCCCACCCC |
| SEQ ID NO: 625 | AACCAATAATGTCCCACCCCG |
| SEQ ID NO: 626 | TACATTGCCCATGTAATTAA |
| SEQ ID NO: 627 | ATATAGTTTCGTCATTCATC |
| SEQ ID NO: 628 | TACATTGCCCATGTAATTAA |
| SEQ ID NO: 629 | ATATAGTTTCGTCATTCATC |
| SEQ ID NO: 630 | AGATAGTTTGGTCATTCATC |
| SEQ ID NO: 631 | AGATAGTTTCGTCATTCATC |
| SEQ ID NO: 632 | AGATAGTTTCGGCATTCATC |
| SEQ ID NO: 633 | AGATAGTTTCGTGATTCATC |
| SEQ ID NO: 634 | ACCAATAATCGCCCACCCCGC |
| SEQ ID NO: 635 | CCAATAATCTGCCACCCCGCC |
| SEQ ID NO: 636 | CAATAATCTCGCACCCCGCCT |
| SEQ ID NO: 637 | AATAATCTCCGACCCCGCCTA |
| SEQ ID NO: 638 | ATAATCTCCCGCCCCGCCTAG |
| SEQ ID NO: 639 | TAATCTCCCAGCCCGCCTAGC |
| SEQ ID NO: 640 | AATCTCCCACGCCGCCTAGCT |
| SEQ ID NO: 641 | ATCTCCCACCGCGCCTAGCTC |
| SEQ ID NO: 642 | TCTCCCACCCGGCCTAGCTCA |
| SEQ ID NO: 643 | CTCCCACCCCGCCTAGCTCAC |
| SEQ ID NO: 644 | TCCCACCCCGGCTAGCTCACG |
| SEQ ID NO: 645 | CCCACCCCGCGTAGCTCACGC |
| SEQ ID NO: 646 | CCACCCCGCCGAGCTCACGCA |
| SEQ ID NO: 647 | CACCCCGCCTGGCTCACGCAA |
| SEQ ID NO: 648 | ACCCCGCCTAGCTCACGCAAG |
| SEQ ID NO: 649 | TACCTCAAAACCCAACTAACC |
| SEQ ID NO: 650 | ACCTCAAAAACCAACTAACCA |
| SEQ ID NO: 651 | CCTCAAAAACCAACTAACCAA |
| SEQ ID NO: 652 | CTCAAAAACCCACTAACCAAC |
| SEQ ID NO: 653 | TCAAAAACCACCTAACCAACC |
| SEQ ID NO: 654 | CAAAAACCAACTAACCAACCA |
| SEQ ID NO: 655 | AAAAACCAACCAACCAACCAA |
| SEQ ID NO: 656 | AAAACCAACTCACCAACCAAT |
| SEQ ID NO: 657 | AACCAACCAACAATCTCCCAC |
| SEQ ID NO: 658 | ACCAACCAATCATCTCCCACC |
| SEQ ID NO: 659 | CCAACCAATACTCTCCCACCC |
| SEQ ID NO: 660 | CAACCAATAACCTCCCACCCC |
| SEQ ID NO: 661 | AACCAATAATCTCCCACCCCG |
| SEQ ID NO: 662 | ATATAGTTTCGTCATTCATC |
| SEQ ID NO: 663 | TACATTGCCCATGTAATTAA |
| SEQ ID NO: 664 | ATATAGTTTCGTCATTCATC |
| SEQ ID NO: 665 | TACATTGCCCATGTAATTAA |
| SEQ ID NO: 666 | AGATAGTTTCGTCATTCATC |
| SEQ ID NO: 667 | AGATAGTTTCCTCATTCATC |
| SEQ ID NO: 668 | AGATAGTTTCGCCATTCATC |
| SEQ ID NO: 669 | AGATAGTTTCGTCATTCATC |
| SEQ ID NO: 670 | ACCAATAATCCCCCACCCCGC |
| SEQ ID NO: 671 | CCAATAATCTCCCACCCCGCC |
| SEQ ID NO: 672 | CAATAATCTCCCACCCCGCCT |
| SEQ ID NO: 673 | AATAATCTCCCACCCCGCCTA |
| SEQ ID NO: 674 | ATAATCTCCCCCCCCGCCTAG |
| SEQ ID NO: 675 | TAATCTCCCACCCCGCCTAGC |
| SEQ ID NO: 676 | AATCTCCCACCCCGCCTAGCT |
| SEQ ID NO: 677 | ATCTCCCACCCCGCCTAGCTC |
| SEQ ID NO: 678 | TCTCCCACCCCGCCTAGCTCA |
| SEQ ID NO: 679 | CTCCCACCCCCCCTAGCTCAC |
| SEQ ID NO: 680 | TCCCACCCCGCCTAGCTCACG |
| SEQ ID NO: 681 | CCCACCCCGCCTAGCTCACGC |
| SEQ ID NO: 682 | CCACCCCGCCCAGCTCACGCA |
| SEQ ID NO: 683 | CACCCCGCCTCGCTCACGCAA |
| SEQ ID NO: 684 | ACCCCGCCTACCTCACGCAAG |
| SEQ ID NO: 685 | TACCTCAAAAACCAACTAACC |
| SEQ ID NO: 686 | ACCTCAAAAAACAACTAACCA |
| SEQ ID NO: 687 | CCTCAAAAACAAACTAACCAA |
| SEQ ID NO: 688 | CTCAAAAACCAACTAACCAAC |
| SEQ ID NO: 689 | TCAAAAACCAACTAACCAACC |
| SEQ ID NO: 690 | CAAAAACCAAATAACCAACCA |
| SEQ ID NO: 691 | AAAAACCAACAAACCAACCAA |
| SEQ ID NO: 692 | AAAACCAACTAACCAACCAAT |
| SEQ ID NO: 693 | AACCAACCAAAAATCTCCCAC |
| SEQ ID NO: 694 | ACCAACCAATAATCTCCCACC |
| SEQ ID NO: 695 | CCAACCAATAATCTCCCACCC |
| SEQ ID NO: 696 | CAACCAATAAACTCCCACCCC |
| SEQ ID NO: 697 | AACCAATAATATCCCACCCCG |
| SEQ ID NO: 698 | TACATTGCCCATGTAATTAA |
| SEQ ID NO: 699 | ATATAGTTTCGTCATTCATC |
| SEQ ID NO: 700 | TACATTGCCCATGTAATTAA |
| SEQ ID NO: 701 | ATATAGTTTCGTCATTCATC |
| SEQ ID NO: 702 | AGATAGTTTAGTCATTCATC |
| SEQ ID NO: 703 | AGATAGTTTCATCATTCATC |
| SEQ ID NO: 704 | AGATAGTTTCGACATTCATC |
| SEQ ID NO: 705 | AGATAGTTTCGTAATTCATC |
| SEQ ID NO: 706 | ACCAATAATCACCCACCCCGC |
| SEQ ID NO: 707 | CCAATAATCTACCACCCCGCC |
| SEQ ID NO: 708 | CAATAATCTCACACCCCGCCT |
| SEQ ID NO: 709 | AATAATCTCCAACCCCGCCTA |
| SEQ ID NO: 710 | ATAATCTCCCACCCCGCCTAG |
| SEQ ID NO: 711 | TAATCTCCCAACCCGCCTAGC |
| SEQ ID NO: 712 | AATCTCCCACACCGCCTAGCT |
| SEQ ID NO: 713 | ATCTCCCACCACGCCTAGCTC |
| SEQ ID NO: 714 | TCTCCCACCCAGCCTAGCTCA |
| SEQ ID NO: 715 | CTCCCACCCCACCTAGCTCAC |
| SEQ ID NO: 716 | TCCCACCCCGACTAGCTCACG |
| SEQ ID NO: 717 | CCCACCCCGCATAGCTCACGC |
| SEQ ID NO: 718 | CCACCCCGCCAAGCTCACGCA |
| SEQ ID NO: 719 | CACCCCGCCTAGCTCACGCAA |
| SEQ ID NO: 720 | ACCCCGCCTAACTCACGCAAG |
| SEQ ID NO: 721 | CCCCGCCTAGTTCACGCAAGC |
| SEQ ID NO: 722 | CCCGCCTAGCTCACGCAAGCC |
| SEQ ID NO: 723 | CCGCCTAGCTTACGCAAGCCG |
| SEQ ID NO: 724 | CGCCTAGCTCTCGCAAGCCGC |
| SEQ ID NO: 725 | GCCTAGCTCATGCAAGCCGCC |
| SEQ ID NO: 726 | CCTAGCTCACTCAAGCCGCCA |
| SEQ ID NO: 727 | CTAGCTCACGTAAGCCGCCAA |
| SEQ ID NO: 728 | TAGCTCACGCTAGCCGCCAAC |
| SEQ ID NO: 729 | AGCTCACGCATGCCGCCAACG |
| SEQ ID NO: 730 | GCTCACGCAATCCGCCAACGC |
| SEQ ID NO: 731 | ATATAGTTTCGTCATTCATC |
| SEQ ID NO: 732 | TACATTGCCCATGTAATTAA |
| SEQ ID NO: 733 | ATATAGTTTCGTCATTCATC |
| SEQ ID NO: 734 | TACATTGCCCATGTAATTAA |
| SEQ ID NO: 735 | AGATAGTTTTGTCATTCATC |
| SEQ ID NO: 736 | AGATAGTTTCTTCATTCATC |
| SEQ ID NO: 737 | AGATAGTTTCGTCATTCATC |
| SEQ ID NO: 738 | AGATAGTTTCGTTATTCATC |
| SEQ ID NO: 739 | CTCACGCAAGTCGCCAACGCC |
| SEQ ID NO: 740 | TCACGCAAGCTGCCAACGCCT |
| SEQ ID NO: 741 | CACGCAAGCCTCCAACGCCTC |
| SEQ ID NO: 742 | ACGCAAGCCGTCAACGCCTCT |
| SEQ ID NO: 743 | CGCAAGCCGCTAACGCCTCTC |
| SEQ ID NO: 744 | GCAAGCCGCCTACGCCTCTCC |
| SEQ ID NO: 745 | CAAGCCGCCATCGCCTCTCCC |
| SEQ ID NO: 746 | AAGCCGCCAATGCCTCTCCCC |
| SEQ ID NO: 747 | AGCCGCCAACTCCTCTCCCCC |
| SEQ ID NO: 748 | GCCGCCAACGTCTCTCCCCCT |
| SEQ ID NO: 749 | CCGCCAACGCTTCTCCCCCTC |
| SEQ ID NO: 750 | CGCCAACGCCTCTCCCCCTCT |
| SEQ ID NO: 751 | GCCAACGCCTTTCCCCCTCTC |
| SEQ ID NO: 752 | CCAACGCCTCTCCCCCTCTCA |
| SEQ ID NO: 753 | CAACGCCTCTTCCCCTCTCAT |
| SEQ ID NO: 754 | AACGCCTCTCTCCCTCTCATC |
| SEQ ID NO: 755 | ACGCCTCTCCTCCTCTCATCC |
| SEQ ID NO: 756 | CGCCTCTCCCTCTCTCATCCA |
| SEQ ID NO: 757 | CCCCGCCTAGGTCACGCAAGC |
| SEQ ID NO: 758 | CCCGCCTAGCGCACGCAAGCC |
| SEQ ID NO: 759 | CCGCCTAGCTGACGCAAGCCG |
| SEQ ID NO: 760 | CGCCTAGCTCGCGCAAGCCGC |
| SEQ ID NO: 761 | GCCTAGCTCAGGCAAGCCGCC |
| SEQ ID NO: 762 | CCTAGCTCACGCAAGCCGCCA |
| SEQ ID NO: 763 | CTAGCTCACGGAAGCCGCCAA |
| SEQ ID NO: 764 | TAGCTCACGCGAGCCGCCAAC |
| SEQ ID NO: 765 | AGCTCACGCAGGCCGCCAACG |
| SEQ ID NO: 766 | GCTCACGCAAGCCGCCAACGC |
| SEQ ID NO: 767 | TACATTGCCCATGTAATTAA |
| SEQ ID NO: 768 | ATATAGTTTCGTCATTCATC |
| SEQ ID NO: 769 | TACATTGCCCATGTAATTAA |
| SEQ ID NO: 770 | ATATAGTTTCGTCATTCATC |
| SEQ ID NO: 771 | AGATAGTTTGGTCATTCATC |
| SEQ ID NO: 772 | AGATAGTTTCGTCATTCATC |
| SEQ ID NO: 773 | AGATAGTTTCGGCATTCATC |
| SEQ ID NO: 774 | AGATAGTTTCGTGATTCATC |
| SEQ ID NO: 775 | CTCACGCAAGGCGCCAACGCC |
| SEQ ID NO: 776 | TCACGCAAGCGGCCAACGCCT |
| SEQ ID NO: 777 | CACGCAAGCCGCCAACGCCTC |
| SEQ ID NO: 778 | ACGCAAGCCGGCAACGCCTCT |
| SEQ ID NO: 779 | CGCAAGCCGCGAACGCCTCTC |
| SEQ ID NO: 780 | GCAAGCCGCCGACGCCTCTCC |
| SEQ ID NO: 781 | CAAGCCGCCAGCGCCTCTCCC |
| SEQ ID NO: 782 | AAGCCGCCAAGGCCTCTCCCC |
| SEQ ID NO: 783 | AGCCGCCAACGCCTCTCCCCC |
| SEQ ID NO: 784 | GCCGCCAACGGCTCTCCCCCT |
| SEQ ID NO: 785 | CCGCCAACGCGTCTCCCCCTC |
| SEQ ID NO: 786 | CGCCAACGCCGCTCCCCCTCT |
| SEQ ID NO: 787 | GCCAACGCCTGTCCCCCTCTC |
| SEQ ID NO: 788 | CCAACGCCTCGCCCCCTCTCA |
| SEQ ID NO: 789 | CAACGCCTCTGCCCCTCTCAT |
| SEQ ID NO: 790 | AACGCCTCTCGCCCTCTCATC |
| SEQ ID NO: 791 | ACGCCTCTCCGCCTCTCATCC |
| SEQ ID NO: 792 | CGCCTCTCCCGCTCTCATCCA |
| SEQ ID NO: 793 | CCCCGCCTAGCTCACGCAAGC |
| SEQ ID NO: 794 | CCCGCCTAGCCCACGCAAGCC |
| SEQ ID NO: 795 | CCGCCTAGCTCACGCAAGCCG |
| SEQ ID NO: 796 | CGCCTAGCTCCCGCAAGCCGC |
| SEQ ID NO: 797 | GCCTAGCTCACGCAAGCCGCC |
| SEQ ID NO: 798 | CCTAGCTCACCCAAGCCGCCA |
| SEQ ID NO: 799 | CTAGCTCACGCAAGCCGCCAA |
| SEQ ID NO: 800 | TAGCTCACGCCAGCCGCCAAC |
| SEQ ID NO: 801 | AGCTCACGCACGCCGCCAACG |
| SEQ ID NO: 802 | GCTCACGCAACCCGCCAACGC |
| SEQ ID NO: 803 | ATATAGTTTCGTCATTCATC |
| SEQ ID NO: 804 | TACATTGCCCATGTAATTAA |
| SEQ ID NO: 805 | ATATAGTTTCGTCATTCATC |
| SEQ ID NO: 806 | TACATTGCCCATGTAATTAA |
| SEQ ID NO: 807 | AGATAGTTTCGTCATTCATC |
| SEQ ID NO: 808 | AGATAGTTTCCTCATTCATC |
| SEQ ID NO: 809 | AGATAGTTTCGCCATTCATC |
| SEQ ID NO: 810 | AGATAGTTTCGTCATTCATC |
| SEQ ID NO: 811 | CTCACGCAAGCCGCCAACGCC |
| SEQ ID NO: 812 | TCACGCAAGCCGCCAACGCCT |
| SEQ ID NO: 813 | CACGCAAGCCCCCAACGCCTC |
| SEQ ID NO: 814 | ACGCAAGCCGCCAACGCCTCT |
| SEQ ID NO: 815 | CGCAAGCCGCCAACGCCTCTC |
| SEQ ID NO: 816 | GCAAGCCGCCCACGCCTCTCC |
| SEQ ID NO: 817 | CAAGCCGCCACCGCCTCTCCC |
| SEQ ID NO: 818 | AAGCCGCCAACGCCTCTCCCC |
| SEQ ID NO: 819 | AGCCGCCAACCCCTCTCCCCC |
| SEQ ID NO: 820 | GCCGCCAACGCCTCTCCCCCT |
| SEQ ID NO: 821 | CCGCCAACGCCTCTCCCCCTC |
| SEQ ID NO: 822 | CGCCAACGCCCCTCCCCCTCT |
| SEQ ID NO: 823 | GCCAACGCCTCTCCCCCTCTC |
| SEQ ID NO: 824 | CCAACGCCTCCCCCCCTCTCA |
| SEQ ID NO: 825 | CAACGCCTCTCCCCCTCTCAT |
| SEQ ID NO: 826 | AACGCCTCTCCCCCTCTCATC |
| SEQ ID NO: 827 | ACGCCTCTCCCCCTCTCATCC |
| SEQ ID NO: 828 | CGCCTCTCCCCCTCTCATCCA |
| SEQ ID NO: 829 | CCCCGCCTAGATCACGCAAGC |
| SEQ ID NO: 830 | CCCGCCTAGCACACGCAAGCC |
| SEQ ID NO: 831 | CCGCCTAGCTAACGCAAGCCG |
| SEQ ID NO: 832 | CGCCTAGCTCACGCAAGCCGC |
| SEQ ID NO: 833 | GCCTAGCTCAAGCAAGCCGCC |
| SEQ ID NO: 834 | CCTAGCTCACACAAGCCGCCA |
| SEQ ID NO: 835 | CTAGCTCACGAAAGCCGCCAA |
| SEQ ID NO: 836 | TAGCTCACGCAAGCCGCCAAC |
| SEQ ID NO: 837 | AGCTCACGCAAGCCGCCAACG |
| SEQ ID NO: 838 | GCTCACGCAAACCGCCAACGC |
| SEQ ID NO: 839 | TACATTGCCCATGTAATTAA |
| SEQ ID NO: 840 | ATATAGTTTCGTCATTCATC |
| SEQ ID NO: 841 | TACATTGCCCATGTAATTAA |
| SEQ ID NO: 842 | ATATAGTTTCGTCATTCATC |
| SEQ ID NO: 843 | AGATAGTTTAGTCATTCATC |
| SEQ ID NO: 844 | AGATAGTTTCATCATTCATC |
| SEQ ID NO: 845 | AGATAGTTTCGACATTCATC |
| SEQ ID NO: 846 | AGATAGTTTCGTAATTCATC |
| SEQ ID NO: 847 | CTCACGCAAGACGCCAACGCC |
| SEQ ID NO: 848 | TCACGCAAGCAGCCAACGCCT |
| SEQ ID NO: 849 | CACGCAAGCCACCAACGCCTC |
| SEQ ID NO: 850 | ACGCAAGCCGACAACGCCTCT |
| SEQ ID NO: 851 | CGCAAGCCGCAAACGCCTCTC |
| SEQ ID NO: 852 | GCAAGCCGCCAACGCCTCTCC |
| SEQ ID NO: 853 | CAAGCCGCCAACGCCTCTCCC |
| SEQ ID NO: 854 | AAGCCGCCAAAGCCTCTCCCC |
| SEQ ID NO: 855 | AGCCGCCAACACCTCTCCCCC |
| SEQ ID NO: 856 | GCCGCCAACGACTCTCCCCCT |
| SEQ ID NO: 857 | CCGCCAACGCATCTCCCCCTC |
| SEQ ID NO: 858 | CGCCAACGCCACTCCCCCTCT |
| SEQ ID NO: 859 | GCCAACGCCTATCCCCCTCTC |
| SEQ ID NO: 860 | CCAACGCCTCACCCCCTCTCA |
| SEQ ID NO: 861 | CAACGCCTCTACCCCTCTCAT |
| SEQ ID NO: 862 | AACGCCTCTCACCCTCTCATC |
| SEQ ID NO: 863 | ACGCCTCTCCACCTCTCATCC |
| SEQ ID NO: 864 | CGCCTCTCCCACTCTCATCCA |
| SEQ ID NO: 865 | GCCTCTCCCCTTCTCATCCAT |
| SEQ ID NO: 866 | CCTCTCCCCCTCTCATCCATC |
| SEQ ID NO: 867 | CTCTCCCCCTTTCATCCATCG |
| SEQ ID NO: 868 | TCTCCCCCTCTCATCCATCGC |
| SEQ ID NO: 869 | CTCCCCCTCTTATCCATCGCC |
| SEQ ID NO: 870 | TCCCCCTCTCTTCCATCGCCC |
| SEQ ID NO: 871 | CCCCCTCTCATCCATCGCCCG |
| SEQ ID NO: 872 | ATATAGTTTCGTCATTCATC |
| SEQ ID NO: 873 | TACATTGCCCATGTAATTAA |
| SEQ ID NO: 874 | ATATAGTTTCGTCATTCATC |
| SEQ ID NO: 875 | TACATTGCCCATGTAATTAA |
| SEQ ID NO: 876 | AGATAGTTTTGTCATTCATC |
| SEQ ID NO: 877 | AGATAGTTTCTTCATTCATC |
| SEQ ID NO: 878 | AGATAGTTTCGTCATTCATC |
| SEQ ID NO: 879 | AGATAGTTTCGTTATTCATC |
| SEQ ID NO: 880 | CCCCTCTCATTCATCGCCCGC |
| SEQ ID NO: 881 | CCCTCTCATCTATCGCCCGCC |
| SEQ ID NO: 882 | CCTCTCATCCTTCGCCCGCCG |
| SEQ ID NO: 883 | CTCTCATCCATCGCCCGCCGC |
| SEQ ID NO: 884 | TCTCATCCATTGCCCGCCGCC |
| SEQ ID NO: 885 | CTCATCCATCTCCCGCCGCCC |
| SEQ ID NO: 886 | TCATCCATCGTCCGCCGCCCC |
| SEQ ID NO: 887 | CATCCATCGCTCGCCGCCCCT |
| SEQ ID NO: 888 | ATCCATCGCCTGCCGCCCCTC |
| SEQ ID NO: 889 | TCCATCGCCCTCCGCCCCTCA |
| SEQ ID NO: 890 | CCATCGCCCGTCGCCCCTCAT |
| SEQ ID NO: 891 | CATCGCCCGCTGCCCCTCATC |
| SEQ ID NO: 892 | ATCGCCCGCCTCCCCTCATCA |
| SEQ ID NO: 893 | TCGCCCGCCGTCCCTCATCAT |
| SEQ ID NO: 894 | CGCCCGCCGCTCCTCATCATA |
| SEQ ID NO: 895 | GCCCGCCGCCTCTCATCATAC |
| SEQ ID NO: 896 | CCCGCCGCCCTTCATCATACC |
| SEQ ID NO: 897 | CCGCCGCCCCTCATCATACCT |
| SEQ ID NO: 898 | CGCCGCCCCTTATCATACCTC |
| SEQ ID NO: 899 | GCCGCCCCTCTTCATACCTCA |
| SEQ ID NO: 900 | CCGCCCCTCATCATACCTCAG |
| SEQ ID NO: 901 | GCCTCTCCCCGTCTCATCCAT |
| SEQ ID NO: 902 | CCTCTCCCCCGCTCATCCATC |
| SEQ ID NO: 903 | CTCTCCCCCTGTCATCCATCG |
| SEQ ID NO: 904 | TCTCCCCCTCGCATCCATCGC |
| SEQ ID NO: 905 | CTCCCCCTCTGATCCATCGCC |
| SEQ ID NO: 906 | TCCCCCTCTCGTCCATCGCCC |
| SEQ ID NO: 907 | CCCCCTCTCAGCCATCGCCCG |
| SEQ ID NO: 908 | TACATTGCCCATGTAATTAA |
| SEQ ID NO: 909 | ATATAGTTTCGTCATTCATC |
| SEQ ID NO: 910 | TACATTGCCCATGTAATTAA |
| SEQ ID NO: 911 | ATATAGTTTCGTCATTCATC |
| SEQ ID NO: 912 | AGATAGTTTGGTCATTCATC |
| SEQ ID NO: 913 | AGATAGTTTCGTCATTCATC |
| SEQ ID NO: 914 | AGATAGTTTCGGCATTCATC |
| SEQ ID NO: 915 | AGATAGTTTCGTGATTCATC |
| SEQ ID NO: 916 | CCCCTCTCATGCATCGCCCGC |
| SEQ ID NO: 917 | CCCTCTCATCGATCGCCCGCC |
| SEQ ID NO: 918 | CCTCTCATCCGTCGCCCGCCG |
| SEQ ID NO: 919 | CTCTCATCCAGCGCCCGCCGC |
| SEQ ID NO: 920 | TCTCATCCATGGCCCGCCGCC |
| SEQ ID NO: 921 | CTCATCCATCGCCCGCCGCCC |
| SEQ ID NO: 922 | TCATCCATCGGCCGCCGCCCC |
| SEQ ID NO: 923 | CATCCATCGCGCGCCGCCCCT |
| SEQ ID NO: 924 | ATCCATCGCCGGCCGCCCCTC |
| SEQ ID NO: 925 | TCCATCGCCCGCCGCCCCTCA |
| SEQ ID NO: 926 | CCATCGCCCGGCGCCCCTCAT |
| SEQ ID NO: 927 | CATCGCCCGCGGCCCCTCATC |
| SEQ ID NO: 928 | ATCGCCCGCCGCCCCTCATCA |
| SEQ ID NO: 929 | TCGCCCGCCGGCCCTCATCAT |
| SEQ ID NO: 930 | CGCCCGCCGCGCCTCATCATA |
| SEQ ID NO: 931 | GCCCGCCGCCGCTCATCATAC |
| SEQ ID NO: 932 | CCCGCCGCCCGTCATCATACC |
| SEQ ID NO: 933 | CCGCCGCCCCGCATCATACCT |
| SEQ ID NO: 934 | CGCCGCCCCTGATCATACCTC |
| SEQ ID NO: 935 | GCCGCCCCTCGTCATACCTCA |
| SEQ ID NO: 936 | CCGCCCCTCAGCATACCTCAG |
| SEQ ID NO: 937 | GCCTCTCCCCCTCTCATCCAT |
| SEQ ID NO: 938 | CCTCTCCCCCCCTCATCCATC |
| SEQ ID NO: 939 | CTCTCCCCCTCTCATCCATCG |
| SEQ ID NO: 940 | TCTCCCCCTCCCATCCATCGC |
| SEQ ID NO: 941 | CTCCCCCTCTCATCCATCGCC |
| SEQ ID NO: 942 | TCCCCCTCTCCTCCATCGCCC |
| SEQ ID NO: 943 | CCCCCTCTCACCCATCGCCCG |
| SEQ ID NO: 944 | ATATAGTTTCGTCATTCATC |
| SEQ ID NO: 945 | TACATTGCCCATGTAATTAA |
| SEQ ID NO: 946 | ATATAGTTTCGTCATTCATC |
| SEQ ID NO: 947 | TACATTGCCCATGTAATTAA |
| SEQ ID NO: 948 | AGATAGTTTCGTCATTCATC |
| SEQ ID NO: 949 | AGATAGTTTCCTCATTCATC |
| SEQ ID NO: 950 | AGATAGTTTCGCCATTCATC |
| SEQ ID NO: 951 | AGATAGTTTCGTCATTCATC |
| SEQ ID NO: 952 | CCCCTCTCATCCATCGCCCGC |
| SEQ ID NO: 953 | CCCTCTCATCCATCGCCCGCC |
| SEQ ID NO: 954 | CCTCTCATCCCTCGCCCGCCG |
| SEQ ID NO: 955 | CTCTCATCCACCGCCCGCCGC |
| SEQ ID NO: 956 | TCTCATCCATCGCCCGCCGCC |
| SEQ ID NO: 957 | CTCATCCATCCCCCGCCGCCC |
| SEQ ID NO: 958 | TCATCCATCGCCCGCCGCCCC |
| SEQ ID NO: 959 | CATCCATCGCCCGCCGCCCCT |
| SEQ ID NO: 960 | ATCCATCGCCCGCCGCCCCTC |
| SEQ ID NO: 961 | TCCATCGCCCCCCGCCCCTCA |
| SEQ ID NO: 962 | CCATCGCCCGCCGCCCCTCAT |
| SEQ ID NO: 963 | CATCGCCCGCCGCCCCTCATC |
| SEQ ID NO: 964 | ATCGCCCGCCCCCCCTCATCA |
| SEQ ID NO: 965 | TCGCCCGCCGCCCCTCATCAT |
| SEQ ID NO: 966 | CGCCCGCCGCCCCTCATCATA |
| SEQ ID NO: 967 | GCCCGCCGCCCCTCATCATAC |
| SEQ ID NO: 968 | CCCGCCGCCCCTCATCATACC |
| SEQ ID NO: 969 | CCGCCGCCCCCCATCATACCT |
| SEQ ID NO: 970 | CGCCGCCCCTCATCATACCTC |
| SEQ ID NO: 971 | GCCGCCCCTCCTCATACCTCA |
| SEQ ID NO: 972 | CCGCCCCTCACCATACCTCAG |
| SEQ ID NO: 973 | GCCTCTCCCCATCTCATCCAT |
| SEQ ID NO: 974 | CCTCTCCCCCACTCATCCATC |
| SEQ ID NO: 975 | CTCTCCCCCTATCATCCATCG |
| SEQ ID NO: 976 | TCTCCCCCTCACATCCATCGC |
| SEQ ID NO: 977 | CTCCCCCTCTAATCCATCGCC |
| SEQ ID NO: 978 | TCCCCCTCTCATCCATCGCCC |
| SEQ ID NO: 979 | CCCCCTCTCAACCATCGCCCG |
| SEQ ID NO: 980 | TACATTGCCCATGTAATTAA |
| SEQ ID NO: 981 | ATATAGTTTCGTCATTCATC |
| SEQ ID NO: 982 | TACATTGCCCATGTAATTAA |
| SEQ ID NO: 983 | ATATAGTTTCGTCATTCATC |
| SEQ ID NO: 984 | AGATAGTTTAGTCATTCATC |
| SEQ ID NO: 985 | AGATAGTTTCATCATTCATC |
| SEQ ID NO: 986 | AGATAGTTTCGACATTCATC |
| SEQ ID NO: 987 | AGATAGTTTCGTAATTCATC |
| SEQ ID NO: 988 | CCCCTCTCATACATCGCCCGC |
| SEQ ID NO: 989 | CCCTCTCATCAATCGCCCGCC |
| SEQ ID NO: 990 | CCTCTCATCCATCGCCCGCCG |
| SEQ ID NO: 991 | CTCTCATCCAACGCCCGCCGC |
| SEQ ID NO: 992 | TCTCATCCATAGCCCGCCGCC |
| SEQ ID NO: 993 | CTCATCCATCACCCGCCGCCC |
| SEQ ID NO: 994 | TCATCCATCGACCGCCGCCCC |
| SEQ ID NO: 995 | CATCCATCGCACGCCGCCCCT |
| SEQ ID NO: 996 | ATCCATCGCCAGCCGCCCCTC |
| SEQ ID NO: 997 | TCCATCGCCCACCGCCCCTCA |
| SEQ ID NO: 998 | CCATCGCCCGACGCCCCTCAT |
| SEQ ID NO: 999 | CATCGCCCGCAGCCCCTCATC |
| SEQ ID NO: 1000 | ATCGCCCGCCACCCCTCATCA |
| SEQ ID NO: 1001 | TCGCCCGCCGACCCTCATCAT |
| SEQ ID NO: 1002 | CGCCCGCCGCACCTCATCATA |
| SEQ ID NO: 1003 | GCCCGCCGCCACTCATCATAC |
| SEQ ID NO: 1004 | CCCGCCGCCCATCATCATACC |
| SEQ ID NO: 1005 | CCGCCGCCCCACATCATACCT |
| SEQ ID NO: 1006 | CGCCGCCCCTAATCATACCTC |
| SEQ ID NO: 1007 | GCCGCCCCTCATCATACCTCA |
| SEQ ID NO: 1008 | CCGCCCCTCAACATACCTCAG |
| SEQ ID NO: 1009 | CGCCCCTCATTATACCTCAGC |
| SEQ ID NO: 1010 | GCCCCTCATCTTACCTCAGCC |
| SEQ ID NO: 1011 | CCCCTCATCATACCTCAGCCG |
| SEQ ID NO: 1012 | CCCTCATCATTCCTCAGCCGC |
| SEQ ID NO: 1013 | ATATAGTTTCGTCATTCATC |
| SEQ ID NO: 1014 | TACATTGCCCATGTAATTAA |
| SEQ ID NO: 1015 | ATATAGTTTCGTCATTCATC |
| SEQ ID NO: 1016 | TACATTGCCCATGTAATTAA |
| SEQ ID NO: 1017 | AGATAGTTTTGTCATTCATC |
| SEQ ID NO: 1018 | AGATAGTTTCTTCATTCATC |
| SEQ ID NO: 1019 | AGATAGTTTCGTCATTCATC |
| SEQ ID NO: 1020 | AGATAGTTTCGTTATTCATC |
| SEQ ID NO: 1021 | CCTCATCATATCTCAGCCGCC |
| SEQ ID NO: 1022 | CTCATCATACTTCAGCCGCCG |
| SEQ ID NO: 1023 | TCATCATACCTCAGCCGCCGC |
| SEQ ID NO: 1024 | CATCATACCTTAGCCGCCGCC |
| SEQ ID NO: 1025 | ATCATACCTCTGCCGCCGCCC |
| SEQ ID NO: 1026 | TCATACCTCATCCGCCGCCCC |
| SEQ ID NO: 1027 | CATACCTCAGTCGCCGCCCCT |
| SEQ ID NO: 1028 | ATACCTCAGCTGCCGCCCCTC |
| SEQ ID NO: 1029 | TACCTCAGCCTCCGCCCCTCA |
| SEQ ID NO: 1030 | ACCTCAGCCGTCGCCCCTCAT |
| SEQ ID NO: 1031 | CCTCAGCCGCTGCCCCTCATC |
| SEQ ID NO: 1032 | CTCAGCCGCCTCCCCTCATCA |
| SEQ ID NO: 1033 | TCAGCCGCCGTCCCTCATCAT |
| SEQ ID NO: 1034 | CAGCCGCCGCTCCTCATCATA |
| SEQ ID NO: 1035 | AGCCGCCGCCTCTCATCATAC |
| SEQ ID NO: 1036 | GCCGCCGCCCTTCATCATACC |
| SEQ ID NO: 1037 | CCGCCGCCCCTCATCATACCT |
| SEQ ID NO: 1038 | CGCCGCCCCTTATCATACCTC |
| SEQ ID NO: 1039 | GCCGCCCCTCTTCATACCTCA |
| SEQ ID NO: 1040 | CCGCCCCTCATCATACCTCAA |
| SEQ ID NO: 1041 | CGCCCCTCATTATACCTCAAA |
| SEQ ID NO: 1042 | GCCCCTCATCTTACCTCAAAA |
| SEQ ID NO: 1043 | CCCCTCATCATACCTCAAAAG |
| SEQ ID NO: 1044 | CCCTCATCATTCCTCAAAAGC |
| SEQ ID NO: 1045 | CGCCCCTCATGATACCTCAGC |
| SEQ ID NO: 1046 | GCCCCTCATCGTACCTCAGCC |
| SEQ ID NO: 1047 | CCCCTCATCAGACCTCAGCCG |
| SEQ ID NO: 1048 | CCCTCATCATGCCTCAGCCGC |
| SEQ ID NO: 1049 | TACATTGCCCATGTAATTAA |
| SEQ ID NO: 1050 | ATATAGTTTCGTCATTCATC |
| SEQ ID NO: 1051 | TACATTGCCCATGTAATTAA |
| SEQ ID NO: 1052 | ATATAGTTTCGTCATTCATC |
| SEQ ID NO: 1053 | AGATAGTTTGGTCATTCATC |
| SEQ ID NO: 1054 | AGATAGTTTCGTCATTCATC |
| SEQ ID NO: 1055 | AGATAGTTTCGGCATTCATC |
| SEQ ID NO: 1056 | AGATAGTTTCGTGATTCATC |
| SEQ ID NO: 1057 | CCTCATCATAGCTCAGCCGCC |
| SEQ ID NO: 1058 | CTCATCATACGTCAGCCGCCG |
| SEQ ID NO: 1059 | TCATCATACCGCAGCCGCCGC |
| SEQ ID NO: 1060 | CATCATACCTGAGCCGCCGCC |
| SEQ ID NO: 1061 | ATCATACCTCGGCCGCCGCCC |
| SEQ ID NO: 1062 | TCATACCTCAGCCGCCGCCCC |
| SEQ ID NO: 1063 | CATACCTCAGGCGCCGCCCCT |
| SEQ ID NO: 1064 | ATACCTCAGCGGCCGCCCCTC |
| SEQ ID NO: 1065 | TACCTCAGCCGCCGCCCCTCA |
| SEQ ID NO: 1066 | ACCTCAGCCGGCGCCCCTCAT |
| SEQ ID NO: 1067 | CCTCAGCCGCGGCCCCTCATC |
| SEQ ID NO: 1068 | CTCAGCCGCCGCCCCTCATCA |
| SEQ ID NO: 1069 | TCAGCCGCCGGCCCTCATCAT |
| SEQ ID NO: 1070 | CAGCCGCCGCGCCTCATCATA |
| SEQ ID NO: 1071 | AGCCGCCGCCGCTCATCATAC |
| SEQ ID NO: 1072 | GCCGCCGCCCGTCATCATACC |
| SEQ ID NO: 1073 | CCGCCGCCCCGCATCATACCT |
| SEQ ID NO: 1074 | CGCCGCCCCTGATCATACCTC |
| SEQ ID NO: 1075 | GCCGCCCCTCGTCATACCTCA |
| SEQ ID NO: 1076 | CCGCCCCTCAGCATACCTCAA |
| SEQ ID NO: 1077 | CGCCCCTCATGATACCTCAAA |
| SEQ ID NO: 1078 | GCCCCTCATCGTACCTCAAAA |
| SEQ ID NO: 1079 | CCCCTCATCAGACCTCAAAAG |
| SEQ ID NO: 1080 | CCCTCATCATGCCTCAAAAGC |
| SEQ ID NO: 1081 | CGCCCCTCATCATACCTCAGC |
| SEQ ID NO: 1082 | GCCCCTCATCCTACCTCAGCC |
| SEQ ID NO: 1083 | CCCCTCATCACACCTCAGCCG |
| SEQ ID NO: 1084 | CCCTCATCATCCCTCAGCCGC |
| SEQ ID NO: 1085 | ATATAGTTTCGTCATTCATC |
| SEQ ID NO: 1086 | TACATTGCCCATGTAATTAA |
| SEQ ID NO: 1087 | ATATAGTTTCGTCATTCATC |
| SEQ ID NO: 1088 | TACATTGCCCATGTAATTAA |
| SEQ ID NO: 1089 | AGATAGTTTCGTCATTCATC |
| SEQ ID NO: 1090 | AGATAGTTTCCTCATTCATC |
| SEQ ID NO: 1091 | AGATAGTTTCGCCATTCATC |
| SEQ ID NO: 1092 | AGATAGTTTCGTCATTCATC |
| SEQ ID NO: 1093 | CCTCATCATACCTCAGCCGCC |
| SEQ ID NO: 1094 | CTCATCATACCTCAGCCGCCG |
| SEQ ID NO: 1095 | TCATCATACCCCAGCCGCCGC |
| SEQ ID NO: 1096 | CATCATACCTCAGCCGCCGCC |
| SEQ ID NO: 1097 | ATCATACCTCCGCCGCCGCCC |
| SEQ ID NO: 1098 | TCATACCTCACCCGCCGCCCC |
| SEQ ID NO: 1099 | CATACCTCAGCCGCCGCCCCT |
| SEQ ID NO: 1100 | ATACCTCAGCCGCCGCCCCTC |
| SEQ ID NO: 1101 | TACCTCAGCCCCCGCCCCTCA |
| SEQ ID NO: 1102 | ACCTCAGCCGCCGCCCCTCAT |
| SEQ ID NO: 1103 | CCTCAGCCGCCGCCCCTCATC |
| SEQ ID NO: 1104 | CTCAGCCGCCCCCCCTCATCA |
| SEQ ID NO: 1105 | TCAGCCGCCGCCCCTCATCAT |
| SEQ ID NO: 1106 | CAGCCGCCGCCCCTCATCATA |
| SEQ ID NO: 1107 | AGCCGCCGCCCCTCATCATAC |
| SEQ ID NO: 1108 | GCCGCCGCCCCTCATCATACC |
| SEQ ID NO: 1109 | CCGCCGCCCCCCATCATACCT |
| SEQ ID NO: 1110 | CGCCGCCCCTCATCATACCTC |
| SEQ ID NO: 1111 | GCCGCCCCTCCTCATACCTCA |
| SEQ ID NO: 1112 | CCGCCCCTCACCATACCTCAA |
| SEQ ID NO: 1113 | CGCCCCTCATCATACCTCAAA |
| SEQ ID NO: 1114 | GCCCCTCATCCTACCTCAAAA |
| SEQ ID NO: 1115 | CCCCTCATCACACCTCAAAAG |
| SEQ ID NO: 1116 | CCCTCATCATCCCTCAAAAGC |
| SEQ ID NO: 1117 | CGCCCCTCATAATACCTCAGC |
| SEQ ID NO: 1118 | GCCCCTCATCATACCTCAGCC |
| SEQ ID NO: 1119 | CCCCTCATCAAACCTCAGCCG |
| SEQ ID NO: 1120 | CCCTCATCATACCTCAGCCGC |
| SEQ ID NO: 1121 | TACATTGCCCATGTAATTAA |
| SEQ ID NO: 1122 | ATATAGTTTCGTCATTCATC |
| SEQ ID NO: 1123 | TACATTGCCCATGTAATTAA |
| SEQ ID NO: 1124 | ATATAGTTTCGTCATTCATC |
| SEQ ID NO: 1125 | AGATAGTTTAGTCATTCATC |
| SEQ ID NO: 1126 | AGATAGTTTCATCATTCATC |
| SEQ ID NO: 1127 | AGATAGTTTCGACATTCATC |
| SEQ ID NO: 1128 | AGATAGTTTCGTAATTCATC |
| SEQ ID NO: 1129 | CCTCATCATAACTCAGCCGCC |
| SEQ ID NO: 1130 | CTCATCATACATCAGCCGCCG |
| SEQ ID NO: 1131 | TCATCATACCACAGCCGCCGC |
| SEQ ID NO: 1132 | CATCATACCTAAGCCGCCGCC |
| SEQ ID NO: 1133 | ATCATACCTCAGCCGCCGCCC |
| SEQ ID NO: 1134 | TCATACCTCAACCGCCGCCCC |
| SEQ ID NO: 1135 | CATACCTCAGACGCCGCCCCT |
| SEQ ID NO: 1136 | ATACCTCAGCAGCCGCCCCTC |
| SEQ ID NO: 1137 | TACCTCAGCCACCGCCCCTCA |
| SEQ ID NO: 1138 | ACCTCAGCCGACGCCCCTCAT |
| SEQ ID NO: 1139 | CCTCAGCCGCAGCCCCTCATC |
| SEQ ID NO: 1140 | CTCAGCCGCCACCCCTCATCA |
| SEQ ID NO: 1141 | TCAGCCGCCGACCCTCATCAT |
| SEQ ID NO: 1142 | CAGCCGCCGCACCTCATCATA |
| SEQ ID NO: 1143 | AGCCGCCGCCACTCATCATAC |
| SEQ ID NO: 1144 | GCCGCCGCCCATCATCATACC |
| SEQ ID NO: 1145 | CCGCCGCCCCACATCATACCT |
| SEQ ID NO: 1146 | CGCCGCCCCTAATCATACCTC |
| SEQ ID NO: 1147 | GCCGCCCCTCATCATACCTCA |
| SEQ ID NO: 1148 | CCGCCCCTCAACATACCTCAA |
| SEQ ID NO: 1149 | CGCCCCTCATAATACCTCAAA |
| SEQ ID NO: 1150 | GCCCCTCATCATACCTCAAAA |
| SEQ ID NO: 1151 | CCCCTCATCAAACCTCAAAAG |
| SEQ ID NO: 1152 | CCCTCATCATACCTCAAAAGC |
| SEQ ID NO: 1153 | CCTCATCATATCTCAAAAGCC |
| SEQ ID NO: 1154 | ATATAGTTTCGTCATTCATC |
| SEQ ID NO: 1155 | TACATTGCCCATGTAATTAA |
| SEQ ID NO: 1156 | ATATAGTTTCGTCATTCATC |
| SEQ ID NO: 1157 | TACATTGCCCATGTAATTAA |
| SEQ ID NO: 1158 | AGATAGTTTTGTCATTCATC |
| SEQ ID NO: 1159 | AGATAGTTTCTTCATTCATC |
| SEQ ID NO: 1160 | AGATAGTTTCGTCATTCATC |
| SEQ ID NO: 1161 | AGATAGTTTCGTTATTCATC |
| SEQ ID NO: 1162 | CTCATCATACTTCAAAAGCCA |
| SEQ ID NO: 1163 | TCATCATACCTCAAAAGCCAA |
| SEQ ID NO: 1164 | CATCATACCTTAAAAGCCAAC |
| SEQ ID NO: 1165 | ATCATACCTCTAAAGCCAACT |
| SEQ ID NO: 1166 | TCATACCTCATAAGCCAACTA |
| SEQ ID NO: 1167 | CATACCTCAATAGCCAACTAA |
| SEQ ID NO: 1168 | ATACCTCAAATGCCAACTAAC |
| SEQ ID NO: 1169 | TACCTCAAAATCCAACTAACC |
| SEQ ID NO: 1170 | ACCTCAAAAGTCAACTAACCA |
| SEQ ID NO: 1171 | CCTCAAAAGCTAACTAACCAA |
| SEQ ID NO: 1172 | CTCAAAAGCCTACTAACCAAC |
| SEQ ID NO: 1173 | TCAAAAGCCATCTAACCAACC |
| SEQ ID NO: 1174 | CAAAAGCCAATTAACCAACCA |
| SEQ ID NO: 1175 | AAAAGCCAACTAACCAACCAA |
| SEQ ID NO: 1176 | AAAGCCAACTTACCAACCAAT |
| SEQ ID NO: 1177 | ATATAGTTTCGTCATTCATC |
| SEQ ID NO: 1178 | TACATTGCCCATGTAATTAA |
| SEQ ID NO: 1179 | ATATAGTTTCGTCATTCATC |
| SEQ ID NO: 1180 | TACATTGCCCATGTAATTAA |
| SEQ ID NO: 1181 | AGATAGTTTTGTCATTCATC |
| SEQ ID NO: 1182 | AGATAGTTTCTTCATTCATC |
| SEQ ID NO: 1183 | AGATAGTTTCGTCATTCATC |
| SEQ ID NO: 1184 | AGATAGTTTCGTTATTCATC |
| SEQ ID NO: 1185 | CCTCATCATAGCTCAAAAGCC |
| SEQ ID NO: 1186 | TACATTGCCCATGTAATTAA |
| SEQ ID NO: 1187 | ATATAGTTTCGTCATTCATC |
| SEQ ID NO: 1188 | TACATTGCCCATGTAATTAA |
| SEQ ID NO: 1189 | ATATAGTTTCGTCATTCATC |
| SEQ ID NO: 1190 | AGATAGTTTGGTCATTCATC |
| SEQ ID NO: 1191 | AGATAGTTTCGTCATTCATC |
| SEQ ID NO: 1192 | AGATAGTTTCGGCATTCATC |
| SEQ ID NO: 1193 | AGATAGTTTCGTGATTCATC |
| SEQ ID NO: 1194 | CTCATCATACGTCAAAAGCCA |
| SEQ ID NO: 1195 | TCATCATACCGCAAAAGCCAA |
| SEQ ID NO: 1196 | CATCATACCTGAAAAGCCAAC |
| SEQ ID NO: 1197 | ATCATACCTCGAAAGCCAACT |
| SEQ ID NO: 1198 | TCATACCTCAGAAGCCAACTA |
| SEQ ID NO: 1199 | CATACCTCAAGAGCCAACTAA |
| SEQ ID NO: 1200 | ATACCTCAAAGGCCAACTAAC |
| SEQ ID NO: 1201 | TACCTCAAAAGCCAACTAACC |
| SEQ ID NO: 1202 | ACCTCAAAAGGCAACTAACCA |
| SEQ ID NO: 1203 | CCTCAAAAGCGAACTAACCAA |
| SEQ ID NO: 1204 | CTCAAAAGCCGACTAACCAAC |
| SEQ ID NO: 1205 | TCAAAAGCCAGCTAACCAACC |
| SEQ ID NO: 1206 | CAAAAGCCAAGTAACCAACCA |
| SEQ ID NO: 1207 | AAAAGCCAACGAACCAACCAA |
| SEQ ID NO: 1208 | AAAGCCAACTGACCAACCAAT |
| SEQ ID NO: 1209 | TACATTGCCCATGTAATTAA |
| SEQ ID NO: 1210 | ATATAGTTTCGTCATTCATC |
| SEQ ID NO: 1211 | TACATTGCCCATGTAATTAA |
| SEQ ID NO: 1212 | ATATAGTTTCGTCATTCATC |
| SEQ ID NO: 1213 | AGATAGTTTGGTCATTCATC |
| SEQ ID NO: 1214 | AGATAGTTTCGTCATTCATC |
| SEQ ID NO: 1215 | AGATAGTTTCGGCATTCATC |
| SEQ ID NO: 1216 | AGATAGTTTCGTGATTCATC |
| SEQ ID NO: 1217 | CCTCATCATACCTCAAAAGCC |
| SEQ ID NO: 1218 | ATATAGTTTCGTCATTCATC |
| SEQ ID NO: 1219 | TACATTGCCCATGTAATTAA |
| SEQ ID NO: 1220 | ATATAGTTTCGTCATTCATC |
| SEQ ID NO: 1221 | TACATTGCCCATGTAATTAA |
| SEQ ID NO: 1222 | AGATAGTTTCGTCATTCATC |
| SEQ ID NO: 1223 | AGATAGTTTCCTCATTCATC |
| SEQ ID NO: 1224 | AGATAGTTTCGCCATTCATC |
| SEQ ID NO: 1225 | AGATAGTTTCGTCATTCATC |
| SEQ ID NO: 1226 | CTCATCATACCTCAAAAGCCA |
| SEQ ID NO: 1227 | TCATCATACCCCAAAAGCCAA |
| SEQ ID NO: 1228 | CATCATACCTCAAAAGCCAAC |
| SEQ ID NO: 1229 | ATCATACCTCCAAAGCCAACT |
| SEQ ID NO: 1230 | TCATACCTCACAAGCCAACTA |
| SEQ ID NO: 1231 | CATACCTCAACAGCCAACTAA |
| SEQ ID NO: 1232 | ATACCTCAAACGCCAACTAAC |
| SEQ ID NO: 1233 | TACCTCAAAACCCAACTAACC |
| SEQ ID NO: 1234 | ACCTCAAAAGCCAACTAACCA |
| SEQ ID NO: 1235 | CCTCAAAAGCCAACTAACCAA |
| SEQ ID NO: 1236 | CTCAAAAGCCCACTAACCAAC |
| SEQ ID NO: 1237 | TCAAAAGCCACCTAACCAACC |
| SEQ ID NO: 1238 | CAAAAGCCAACTAACCAACCA |
| SEQ ID NO: 1239 | AAAAGCCAACCAACCAACCAA |
| SEQ ID NO: 1240 | AAAGCCAACTCACCAACCAAT |
| SEQ ID NO: 1241 | ATATAGTTTCGTCATTCATC |
| SEQ ID NO: 1242 | TACATTGCCCATGTAATTAA |
| SEQ ID NO: 1243 | ATATAGTTTCGTCATTCATC |
| SEQ ID NO: 1244 | TACATTGCCCATGTAATTAA |
| SEQ ID NO: 1245 | AGATAGTTTCGTCATTCATC |
| SEQ ID NO: 1246 | AGATAGTTTCCTCATTCATC |
| SEQ ID NO: 1247 | AGATAGTTTCGCCATTCATC |
| SEQ ID NO: 1248 | AGATAGTTTCGTCATTCATC |
| SEQ ID NO: 1249 | CCTCATCATAACTCAAAAGCC |
| SEQ ID NO: 1250 | TACATTGCCCATGTAATTAA |
| SEQ ID NO: 1251 | ATATAGTTTCGTCATTCATC |
| SEQ ID NO: 1252 | TACATTGCCCATGTAATTAA |
| SEQ ID NO: 1253 | ATATAGTTTCGTCATTCATC |
| SEQ ID NO: 1254 | AGATAGTTTAGTCATTCATC |
| SEQ ID NO: 1255 | AGATAGTTTCATCATTCATC |
| SEQ ID NO: 1256 | AGATAGTTTCGACATTCATC |
| SEQ ID NO: 1257 | AGATAGTTTCGTAATTCATC |
| SEQ ID NO: 1258 | CTCATCATACATCAAAAGCCA |
| SEQ ID NO: 1259 | TCATCATACCACAAAAGCCAA |
| SEQ ID NO: 1260 | CATCATACCTAAAAAGCCAAC |
| SEQ ID NO: 1261 | ATCATACCTCAAAAGCCAACT |
| SEQ ID NO: 1262 | TCATACCTCAAAAGCCAACTA |
| SEQ ID NO: 1263 | CATACCTCAAAAGCCAACTAA |
| SEQ ID NO: 1264 | ATACCTCAAAAGCCAACTAAC |
| SEQ ID NO: 1265 | TACCTCAAAAACCAACTAACC |
| SEQ ID NO: 1266 | ACCTCAAAAGACAACTAACCA |
| SEQ ID NO: 1267 | CCTCAAAAGCAAACTAACCAA |
| SEQ ID NO: 1268 | CTCAAAAGCCAACTAACCAAC |
| SEQ ID NO: 1269 | TCAAAAGCCAACTAACCAACC |
| SEQ ID NO: 1270 | CAAAAGCCAAATAACCAACCA |
| SEQ ID NO: 1271 | AAAAGCCAACAAACCAACCAA |
| SEQ ID NO: 1272 | AAAGCCAACTAACCAACCAAT |
| SEQ ID NO: 1273 | TACATTGCCCATGTAATTAA |
| SEQ ID NO: 1274 | ATATAGTTTCGTCATTCATC |
| SEQ ID NO: 1275 | TACATTGCCCATGTAATTAA |
| SEQ ID NO: 1276 | ATATAGTTTCGTCATTCATC |
| SEQ ID NO: 1277 | AGATAGTTTAGTCATTCATC |
| SEQ ID NO: 1278 | AGATAGTTTCATCATTCATC |
| SEQ ID NO: 1279 | AGATAGTTTCGACATTCATC |
| SEQ ID NO: 1280 | AGATAGTTTCGTAATTCATC |

Procedure for Probe Design

The design of a probe begins with the input of a sequence file into a computer in the five prime to three prime direction. The sequence file is then converted to account for sodium bisulfite treatment. The complementary sequence of the converted sequence file is then is then generated in the three prime to five prime direction.

A parent probe list is then created from the complementary sequence. This is accomplished by standard re-sequencing, where every base is queried. For this method the first probe starts at position X, and extend a number of bases, N. The next probe starts at position X+1, and extends N bases also. A second method to create the parent probe set is to identify all CpG dinucleotides and only create probes with a CpG dinucleotide in the middle.

Once prepared, the parent probe list is filtered to remove probes that are deemed not to be suitable for re-sequencing analysis. Factors such as low sequence complexity are taken into account. Each parent probe is used as a template to create new probes to query for possible changes at a particular position in the reference sequence. Each parent probe generates at least three new probes, one for each single nucleotide polymorphism at the central base. The parent probe and daughter probes created from it represent the position query probe partners. Additional position query probe partners may be required if multiple CpG islands are on one probe. In this case every possible combination of methylation sites from the parent probe must be created. This creates a list of sub parent probes each of whose central position is then altered to represent all possible single nucleotide polymorphisms. The collection of these probes are that position's position query probe partners.

Once the complete set of position query probe partners has been calculated, a file is generated containing all the partners for each position in the reference sequence, or those designated by the user for interrogation. A probe set generated in this manner for a portion of p16 is attached as Appendix 1.

Sodium Bisulfite Treatment Protocol

The concentration of DNA used in this protocol is 1 μg of DNA per 10 μl of sample. Samples are prepared in an autoclaved tube with 1 μg of DNA diluted to 50 μl using autoclaved water. 5.5 μl of 2M sodium hydroxide (3.6 g in 45 ml of water) is then added and the sample is maintained at 37° C. for ten minutes in a water bath. The sample tube is removed from the water bath and centrifuged. 30 μl of freshly prepared hydroquinone solution (55 mg in 50 ml of water) is added to the sample tube and the sample becomes yellow. 520 μl of freshly prepared sodium bisulfite solution (3.76 g in 10 ml of water) is then added and the resulting solution is mixed well. The sample tube is then sealed with parafilm and placed in a water bath at 60° C. for 16 hours. The tubes are removed from the water bath and the sample purified using the Wizard DNA resin (Promega) according to the manufacturer's protocol. The DNA is eluted with 50 μl of water to which is added 8.25 μl of 2M sodium hydroxide solution. The DNA is then precipitated using ethanol and a glycogen carrier. The precipitated DNA is then resuspended in 200 μl of water.

Protocol for PCR Amplification of 145 bp Region of the Promoter for p16

The primers listed below are examples of those used for the amplification.

| | |
|---|---|
| Primer Sequences: | |
| 5′ (Cy3/Cy5) GTTTTCCCAGTCACGACTTGGTTGGTTATTAGAGGGTGG 3′ | (SEQ ID NO.: 1281) |
| 5′ (Cy3/Cy5) AAACAGCTATGACCATGACCATAACCAACCAATCAACC 3′ | (SEQ ID NO.: 1282) |
| The entire 145 base sequence: | |
| 5′CTGGCTG GTCACCAGAGGGTGGGGCGG ACCGAGTGCG CTCGGCGGCT | (SEQ ID NO.: 1283) |
| GCGGAGAGGG GTAGAGCAGG CAGCGGGCGGCGGGGAGCAG CATGGAGCCG | |
| GCGGCGGGGA GCAGCATGGA GCCTTCGGCT GACTGGCTGG CCACGGC3′ | |

The following procedure is typically done 50 times, and the resulting material combined to form a single sample. Each amplification is accomplished by adding 3.2 μl of dNTP mixture (1.25 μM in each base), 2.5 μl of 10×PCR buffer, 1 μl of primer mixture (25 μM for each primer), 17 μl of water, 0.2 μl Taq polymerase (5 units/μl) and 1 μl of template DNA from the bisulfite treatment protocol described above.

The thermocycler is then programmed to 95° C. for 12 minutes. This is followed by two cycles of treatment at 94° C. for 20 seconds, 66° C. for 40 seconds and 72° C. for 20 seconds with touchdown of −1° C. This is followed by 35 cycles of treatment at 94° C. for 20 seconds, 66° C. for 30 seconds and 72° C. for 20 seconds with touchdown of −1° C. The sample is then kept at 72° C. got 7 minutes and stored at 4° C.

### EXAMPLE 2

### Analysis of Methylation of a Region of the Promoter for the Tumor Suppressor Gene p16 with Oligonucleotide Arrays

An example of a method for mapping individual sites of CpG methylation in genomic DNA is further presented herein. The method of the present invention allows parallel and simultaneous analysis of many individual potential sites of methylation in widely separated regions of the genome.

Array Fabrication

Corning 1″×3″ glass microscope slides were cleaned and coated with 3-glycidoxypropyltrimethoxysilane (Aldrich) and polyethlyeneglycol (Mₐ 300, Aldrich) as described by Maskos and Southern. Slides were stored in a dessicator at room temperature until use. In preparation for microarray fabrication, the synthesis area of a slide was reacted with a 1:1 (vol:vol) mixture of 0.1 M protected linker phosphoramidite (MeNPOC-hexaethylene glycol β-cyanoethyl phosphoramidite) and tetrazole in acetonitrile (Annovis, Aston, Pa.). The mixture was allowed to react for two minutes with the glass surface and then washed with acetonitrile.

An array of oligonucleotide probes was synthesized in situ on the resulting surface using light directed phosphoramidite synthesis. MenPOC-protected phosphoramidites were used in the synthesis. Light for each photochemical deprotection step was spatially addressed with a Texas Instruments Digital Light Processor (DLP™). The DLP was illuminated with the 365 nm peak from a 200 W Hg/Xe arc lamp. Illumination of the DLP and projection of the reflected image were accomplished with a custom optical system designed by Brilliant Technologies (Denton, Tex.). The image of the DLP was projected onto the reactive surface without magnification. The DLP was coordinated with a home-built fluidics system for automated DNA synthesis. Custom software generated the patterns of illumination required to fabricate the desired array of oligonucleotides. Final deprotection of the synthesized array was with a 1:1 (vol:vol) solution of ethylenediamine and ethanol for two hours at room temperature.

Preparation of DNA and Amplification of Promoter Regions

Cell lines H1299 and H69 were established as described by Phelps and co-workers (Phelps R, Johnson B, Ihde D, et al., NCI-Navy medical oncology branch cell line data base, Journal of Cellular Biochemistry Supplement. 24: 32-91, 1996) and have been deposited in the American Type Culture Collection. The cells were cultured in RPMI 1640 (Invitrogen) supplemented with 5% fetal bovine serum. Genomic DNA was purified from these cell lines as described by Fong et al. (Fong L, Zimmerman P, and Smith P, Correlation of loss of heterozygosity at 11 p with tumour progression and survival in non-small cell lung cancer, Genes, Chromosomes, Cancer. 10: 183-189, 1994). The extracted, purified DNA was treated with sodium bisulfite. Thep16 promoter region was amplified in a PCR reaction using 50 ng sodium bisulfite-treated genomic DNA as template and the following primers: 5′[Cy3 or biotin] TTAGAGGATTTGAGGGAT3′ (SEQ ID NO.: 1284) and 5′AAAACTCCATACTACTCC 3′ (SEQ ID NO.: 1285). Primers were purchased from Operon Technologies (Alameda, Calif.).

A touchdown method was used for the first 14 cycles of amplification, starting at an annealing temperature of 68° C. and decreasing the annealing temperature 1° C. per cycle. Amplification was continued for an additional 30 cycles with an annealing temperature of 55° C. Denaturation and extension were carried out at 94° C. and 72° C., respectively. The product of this amplification was used as the template for a second set of PCR reactions. The products were de-salted (NAP column, Amersham Pharmacia Biotech) and precipitated with ethanol and sodium acetate prior to dissolving in hybridization buffer.

Array Hybridization

The hybridization mixture contained, 0.1-1 μM labeled analyte sample, 0.1-1 μM labeled reference sample, 1 μM Control Oligo 1 (SEQ ID NO.: 1286, 5′[Cy3] CTTGGCTGTCCCAGAATGCAAGAAGCCCAGACGGAAACCGTAGCTGCCCTGGTA GGTTTT), and 1 μM Control Oligo 2 (SEQ ID NO.: 1287, 5′[Cy3] TATATCAAAGCAGTAAGTAG) in 3M tetramethyl ammonium chloride, 0.05% Trition X-100,1 mM EDTA, 10 mM Tris HCl pH7.5. The sample was applied to the array surface under a 22×22 mm cover slip. Hybridization was carried out in a closed chamber containing a pool of hybridization buffer. The array with sample was heated to 95° C. for 20 minutes followed by warming at 60° C. for one hour. After hybridization, the array was washed three times with 6×SSPE (Sigma), 0.09% Tween, followed by three washes with 0.8×SSPE, 0.01% Tween at room temperature. After this wash, the array was dried centrifugally, stained with 2 μg/ml of CyS-Streptavidin (vendor) for 5 minutes at room temperature, washed with 6×SSPE, 0.09% Tween. Finally, the array was scanned using an Axon Genepix 3000 scanner to detect Cy3 and Cy5 fluorescence intensity. The signal intensity for each feature was determined using custom analysis software.

TA Cloning and Sequencing

The 190 base pair amplicon of sodium bisulfite treated DNA was cloned into plasmid pCR®2.1 using a TA cloning kit (Invitrogen, Carlsbad, Calif.) and manufacturer recommended protocols. Plasmid was isolated from 18 individual colonies, and the insert was sequenced. Sequencing was done on an ABI3100 sequencer with T7 and M13 primers using dye terminated DNA sequencing protocols.

Construction of 190 bp Duplex for Heterogeneous Methylation Study

A 190 base pair duplex with simulated methylation at position 25 was created. Oligonucleotides were obtained from Operon Technologies. The following oligonucleotides were obtained from Operon Technologies: Oligo A (SEQ ID NO.: 1288, 5′CCACCCTCTAATAACCAACCAACCCCTCCTCTTTCTTCCTCCAATACTAACAAA AAAACCCCCTCCAACCCTATCCCTCAAATCCTCTAA), Oligo B (SEQ ID NO.: 1289, 5′GTGTGTTTGGTGGTTGCGGAGAGGGGGAGAGTAGGTAGTGGGTGGTGGGGAGT AGTATGGAGTTGGTGGTGGGGAGTAGTATGGAGTTTT), Oligo C (SEQ ID NO.: 1290, 5′TTAGAGGATTTGAGGGATAGGGTTGGAGGGGGTTTTTTTGTTAGTATTGGAGG AAGAAAGAGGAGGGGTTGGTTGGTTATTAGAGGGTGGGGTGGATTGT), and Oligo D (SEQ ID NO.: 1291, 5′AAAACTCCATACTACTCCCCACCACCAACTCCATA CTACTCCCCACCACCCACTACCTACTCTCCCCCTCTCCGCAACCACCAAACACAC ACAATCCACC). Oligos A and B (70 pmoles each) were phosphorylated with polynucleotide kinase (New England BioLabs). The phosphorylated DNA was phenol extracted, chloroform extracted, then ethanol precipitated. Phosphorylated Oligo A was annealed with Oligo C, and phosphorylated Oligo B was annealed with Oligo D. The resulting duplexes were mixed in equimolar amounts and ligated with T4 ligase at 14° C. overnight. The resulting 190 base pair duplex was amplified as described above for the p16 promoter region.

Assay for Methylation by Hybridization to an Array of Oligonucleotide Probes

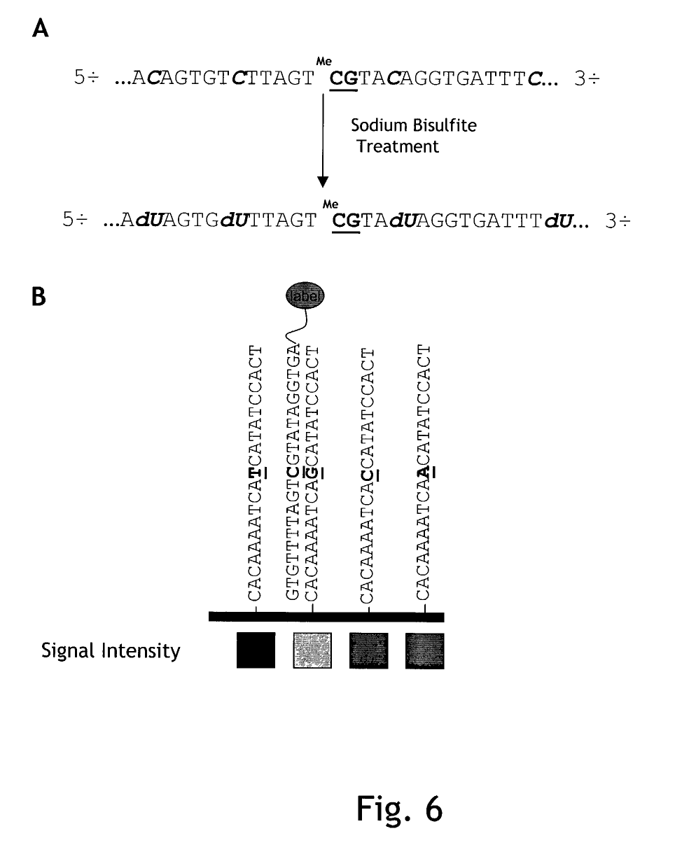

An example of one ore more essential features of the present invention is shown schematically in FIG. 6. For FIG. 6, oligonucleotide probes are covalently bound to a substrate. The central base of each probe for a given position is varied to test for the identity of the base by hybridization. The probe with which the most label is associated identifies the base at the central position. A cytosine at the probed position indicates methylation that prevented conversion by sodium bisulfite. A sample of genomic DNA is treated with sodium bisulfite under conditions that convert unmethylated cytosines to deoxyuridines. Methylated cytosines remain unconverted (FIG. 6A). At least one region of interest is amplified by PCR, which recapitulates the deoxyuracils in the template as thymidines. The product is labeled during amplification with an easily detectable tag such as a fluorophore. The presence of a cytosine or a thymidine at each position corresponding to a site of potential methylation is assayed by hybridization to a set of complementary oligonucleotide probes covalently bound to a substrate (FIG. 6B). Each probe for a given position is identical, except for a center base substitution used to determine the analyte sequence by hybridization. Many different CpG sites may be simultaneously queried with an array of many oligonucleotide probes.

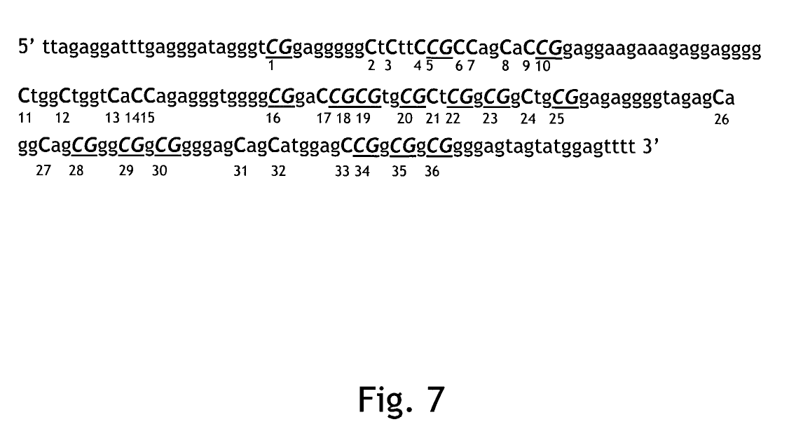

A region of the promoter for the tumor suppressor gene p16 is tested using the method of the present invention. Hypermethylation of this promoter is known to repress transcription of p16 and is associated with a number of cancers. Samples of genomic DNA from lung tumor cell lines are treated with sodium bisulfite. In addition, a190 bp region of the p16 promoter is amplified and labeled. The sequence of the 190 base region of interest (prior to treatment with sodium bisulfite) is shown in FIG. 7 (GenBank accession number AL449423). After treatment with bisulfite, the strand shown was amplified and labeled. The region contains 36 cytosines. The numbers correspond to those are depicted in TABLE 2; 16 cytosines are within CpG dinucleotides (shaded) and 20 cytosines are not within CpG dinucleotides. The amplified DNA was analyzed by hybridization to an array of oligonucleotide probes, each 21 bases in length, synthesized directly on a glass surface by light-directed methods. Spatially patterned illumination for the photodeprotection step of the synthesis was accomplished using a digital micromirror device.

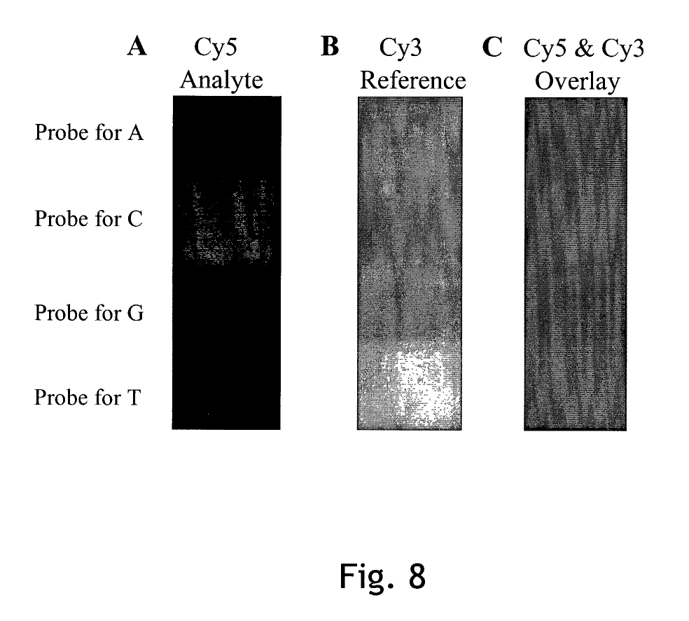

The result of hybridization and scanning of four probes designed to query a single cytosine (cytosine number 1) is shown in FIG. 8. The array was hybridized, washed, and scanned for fluorescence. Each 21 -nucleotide probe is complementary to the sequence surrounding cytosine number 1, with a different base for each probe in apposition to cytosine number 1. For example, the probe for A has a thymidine in that central position. The DNA analyzed with the Cy5 label was from a lung tumor cell line (H1299) in which all of the CpG dinucleotides in the 190-base analyzed region were previously found to be methylated (by using dye terminated sequencing of bisulfite treated DNA). The feature with the highest signal of the four features shown is the one probing for a cytosine (the variable base in the probe is a guanine). The ratio of the signal for this feature to the next highest signal (in the feature probing for a guanine) is 2.8, identifying the base in the analyte as a cytosine. A cytosine at this position was anticipated as the outcome of bisulfite treatment of the methylated base.

One comparison relevant to detection of methylation is between the signal in the feature that probes for a cytosine at each position and the signal in the feature that probes for a thymidine at the same position in the bisulfite treated DNA. The ratio of these signals (C:T) is listed for each of the cytosines in the analyzed sequence in TABLE 2. Cytosines outside of CpG dinucleotides that are not methylated serve as an internal indicator for the effectiveness of the bisulfite treatment in converting unmethylated cytosines to deoxyuracils and for the discrimination between cytosines and thymidines by the probes on the array. The ratio of signals in those features ranges from 0.24 to 1.09. Independent sequence analysis of the bisulfite-treated DNA confirmed complete conversion of all unmethylated cytosines to deoxyuracils. At the position queried by the probes shown in FIG. 8, the ratio of signals (C:T) is 3.57. The values range from 1.91 to 13.8 for cytosines in CpG dinucleotides (TABLE 2), in all cases considerably higher than the highest ratio of signals for the unmethylated cytosines.

TABLE 2
Summary of Signal Intensity Ratios for Each Analyzed Cytosine

| | H1299 & H69^{d} | | | | 25th C Duplex^{e} | | | |
|---|---|---|---|---|---|---|---|---|
| Cytosine | C:T Ratio | C:T Ratio | Analyte(C:T)/ | | C:T Ratio | C:T Ratio | Analyte(C:T)/ | |
| Number^{g} | Analyte^{a} | Reference^{a} | Ref(C:T)^{b} | Score^{c} | Analyte^{a} | Reference^{a} | Ref(C:T)^{b} | Z Score^{c} |
| 1 | 3.57 | 0.52 | 6.80 | 10.7 | 0.86 | 0.88 | 0.99 | −0.90 |
| 2 | 0.46 | 0.54 | 0.85 | −1.50 | 0.74 | 0.69 | 1.08 | −0.29 |
| 3 | 0.44 | 0.36 | 1.23 | −0.72 | 0.75 | 0.75 | 1.00 | −0.82 |
| 4 | 0.39 | 0.29 | 1.34 | −0.50 | 0.87 | 0.86 | 1.01 | −0.76 |
| 5 | 13.8 | 0.39 | 35.7 | 69.7 | 0.90 | 0.89 | 1.01 | −0.75 |
| 6 | 0.24 | 0.22 | 1.13 | −0.94 | 1.07 | 0.96 | 1.12 | −0.08 |
| 7 | 0.34 | 0.36 | 0.94 | −1.33 | 1.01 | 0.99 | 1.01 | −0.72 |
| 8 | 0.36 | 0.41 | 0.88 | −1.45 | 0.70 | 0.58 | 1.22 | 0.58 |
| 9 | 0.33 | 0.27 | 1.23 | −0.73 | 0.68 | 0.65 | 1.05 | −0.50 |
| 10 | 9.28 | 0.41 | 22.5 | 42.8 | 0.82 | 0.68 | 1.20 | 0.46 |
| 11 | 0.93 | 0.53 | 1.76 | 0.36 | 0.85 | 0.88 | 0.97 | −1.00 |
| 12 | 1.09 | 0.48 | 2.29 | 1.44 | 1.01 | 0.72 | 1.41 | 1.79 |
| 13 | 0.65 | 0.52 | 1.23 | −0.69 | 0.85 | 0.76 | 1.11 | −0.10 |
| 14 | 0.65 | 0.51 | 1.23 | −0.60 | 0.83 | 0.80 | 1.05 | −0.52 |
| 15 | 1.08 | 0.60 | 1.81 | 0.44 | 0.92 | 0.93 | 0.99 | −0.87 |
| 16 | 3.55 | 0.54 | 6.64 | 10.3 | 0.94 | 0.72 | 1.30 | 1.12 |
| 17 | 0.27 | 0.11 | 2.44 | 1.75 | 0.62 | 0.56 | 1.11 | −0.11 |
| 18 | 1.99 | 0.46 | 4.34 | 5.62 | 0.9 | 1.06 | 0.85 | −1.76 |
| 19 | 2.36 | 0.60 | 3.91 | 4.75 | 1.10 | 0.76 | 1.45 | 2.08 |
| 20 | 1.91 | 0.53 | 3.63 | 4.18 | 1.01 | 0.82 | 1.23 | 0.68 |
| 21 | 0.40 | 0.18 | 2.27 | 1.39 | 0.51 | 0.45 | 1.14 | 0.08 |
| 22 | 3.11 | 0.69 | 4.54 | 6.05 | 0.82 | 0.71 | 1.16 | 0.24 |
| 23 | 3.38 | 0.59 | 5.73 | 8.46 | 1.07 | 0.68 | 1.56 | 2.77 |
| 24 | 0.45 | 0.27 | 1.68 | 0.20 | 0.60 | 0.49 | 1.22 | 0.62 |
| 25 | 3.55 | 0.52 | 6.81 | 10.7 | 1.48 | 0.62 | 2.38 | 7.97 |
| 26 | 0.62 | 0.29 | 2.11 | 1.07 | 0.81 | 0.75 | 1.08 | −0.29 |
| 27 | 0.46 | 0.29 | 1.58 | −0.01 | 0.7 | 0.74 | 0.94 | −1.17 |
| 28 | 2.88 | 0.52 | 5.52 | 8.02 | 1.00 | 0.89 | 1.12 | −0.04 |
| 29 | 2.11 | 0.43 | 4.85 | 6.66 | 0.93 | 0.58 | 1.59 | 2.95 |
| 30 | 3.40 | 0.42 | 8.09 | 13.3 | 1.01 | 0.62 | 1.67 | 3.47 |
| 31 | 0.70 | 0.38 | 1.87 | 0.57 | 0.77 | 0.58 | 1.32 | 1.23 |
| 32 | 0.60 | 0.34 | 1.75 | 0.33 | 0.79 | 0.50 | 1.57 | 2.82 |
| 33 | 0.37 | 0.18 | 2.04 | 0.93 | 0.57 | 0.50 | 1.14 | 0.09 |
| 34 | 2.14 | 0.52 | 4.10 | 5.13 | 0.82 | 0.63 | 1.30 | 1.09 |
| 35 | 2.11 | 0.44 | 4.77 | 6.51 | 1.21 | 0.72 | 1.69 | 3.55 |
| 36 | 4.48 | 0.49 | 9.15 | 15.5 | 1.18 | 0.80 | 1.47 | 2.20 |

| | 20:80 Mixture^{f} | | | |
|---|---|---|---|---|
| Cytosine | C:T Ratio | C:T Ratio | Analyte(C:T)/ | |
| Number^{g} | Analyte^{a} | Reference^{a} | Ref(C:T)^{b} | Z Score^{c} |
| 1 | 0.99 | 0.52 | 1.92 | 4.61 |
| 2 | 0.70 | 0.70 | 1.00 | 1.01 |
| 3 | 0.39 | 0.32 | 1.20 | 1.80 |
| 4 | 0.44 | 0.36 | 1.22 | 1.88 |
| 5 | 1.16 | 0.49 | 2.35 | 6.29 |
| 6 | 0.32 | 0.64 | 0.5 | −0.97 |
| 7 | 0.50 | 0.76 | 0.65 | −0.37 |
| 8 | 0.36 | 0.62 | 0.58 | −0.64 |
| 9 | 0.34 | 0.64 | 0.53 | −0.85 |
| 10 | 1.43 | 0.67 | 2.15 | 5.51 |
| 11 | 0.62 | 0.90 | 0.69 | −0.20 |
| 12 | 0.70 | 0.55 | 1.28 | 2.08 |
| 13 | 0.61 | 0.93 | 0.66 | −0.35 |
| 14 | 0.51 | 0.68 | 0.74 | −0.02 |
| 15 | 0.61 | 0.98 | 0.62 | −0.48 |
| 16 | 1.90 | 0.86 | 2.21 | 5.71 |
| 17 | 0.20 | 0.51 | 0.39 | −1.41 |
| 18 | 0.50 | 0.42 | 1.19 | 1.73 |
| 19 | 1.04 | 0.57 | 1.83 | 4.25 |
| 20 | 1.99 | 1.04 | 1.92 | 4.58 |
| 21 | 0.35 | 0.62 | 0.57 | 0.69 |
| 22 | 2.17 | 1.39 | 1.56 | 3.19 |
| 23 | 2.20 | 1.41 | 1.59 | 3.32 |
| 24 | 0.34 | 0.49 | 0.70 | 0.17 |
| 25 | 1.12 | 0.74 | 1.51 | 2.99 |
| 26 | 0.69 | 0.78 | 0.89 | 0.59 |
| 27 | 0.49 | 0.87 | 0.56 | −0.73 |
| 28 | 1.24 | 0.63 | 1.98 | 4.82 |
| 29 | 0.93 | 0.96 | 0.96 | 0.85 |
| 30 | 0.91 | 1.11 | 0.82 | 0.29 |
| 31 | 0.59 | 0.73 | 0.81 | 0.25 |
| 32 | 0.53 | 0.67 | 0.80 | 0.21 |
| 33 | 0.30 | 0.59 | 0.51 | −0.93 |
| 34 | 1.16 | 0.63 | 1.85 | 4.33 |
| 35 | 1.31 | 1.33 | 0.98 | 0.93 |
| 36 | 2.28 | 1.66 | 1.38 | 2.48 |

^{a}mean fluorescence signal in the region defined by the probe for cytosine at a position divided by the mean fluorescence signal in the region defined by the probe for thymidine at the same position.
^{b}C:T ratio at a probed position for the fluorescence channel corresponding to the analyte sample divided by the C:T ratio at the same position for the fluorescence channel corresponding to the reference sample.
^{c}Z score = (R − Rᵤ)/S, where R = analyte(C:T)/reference(C:T) at a given position, Rᵤ = mean analyte(C:T)/reference(C:T) for all cytosines not in CpGs, and S = standard deviation in the mean analyte(C:T)/reference(C:T) for all cytosines not in CpGs.
^{d}Analysis of sample derived from fully methylated DNA from lung tumor cell line H1299 with reference derived from unmethylated DNA from lung tumor cell line H69.
^{e}Analysis in which the analyte was derived from a synthetic duplex simulating unique methylation at cytosine number 25.
^{f}Analysis in which the analyte was derived from a mixture of approximately 20% methylated DNA and 80% unmethylated DNA.
^{g}Cytosines in CpG islands are in shaded rows.

To provide an objective standard for discrimination between methylated and unmethylated cytosines and to facilitate visualization of changes in methylation state, a reference sequence containing a different label was co-hybridized with the array. DNA from a different lung tumor cell line (H69) in which the p16 promoter has been found to be unmethylated at each CpG in the 190 base region of interest was used a model reference sequence. Results were confirmed using dye terminated sequencing of bisulfite-treated DNA. The same 190 base region (FIG. 7) of H69 was amplified with a primer labeled with Cy3.

The result for cytosine number 1 is shown in FIGS. 8B and 8C. The probe for thymidine has the highest signal intensity, and the C:T ratio for the reference strand is 0.52 at this position. A useful method for judging changes in methylation state is to compare the C:T ratio for a set of probes with the analyte fluorophore to the C:T ratio for the same probes with the reference fluorophore. In FIG. 8 the ratio of sample fluorophore (Cy5) C:T ratio to reference fluorophore (Cy3) C:T ratio is 6.8. Using a ratio of ratios in this manner may, for example, reduce the effects of imperfect hybridization specificity on the results.

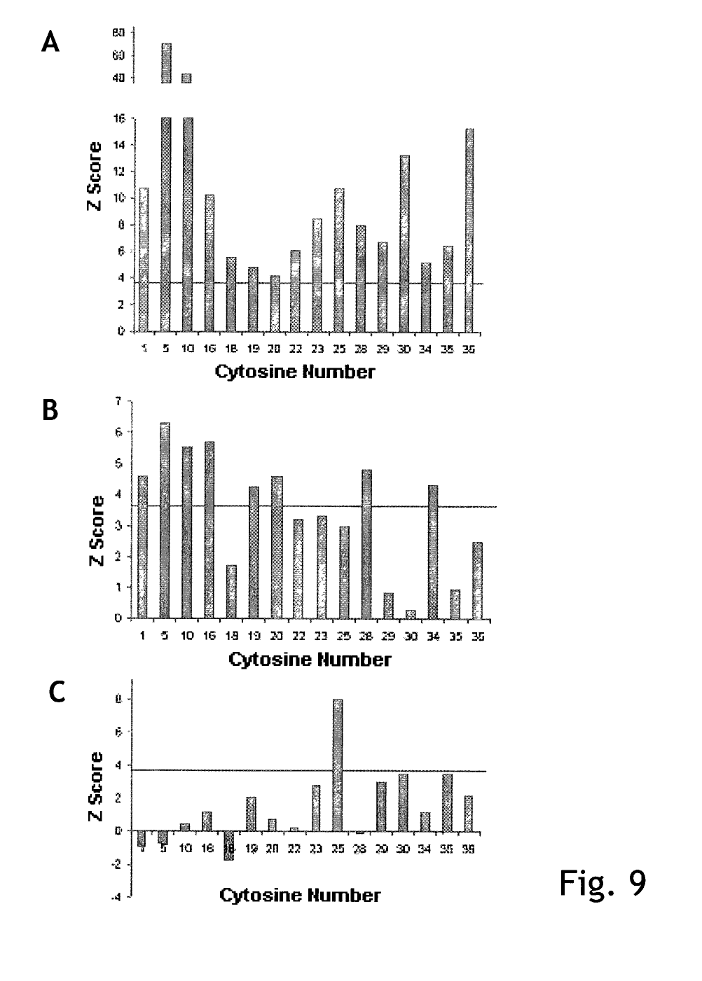

The ratio of ratios was computed for each cytosine in the original sequence and is listed in TABLE 2. Cytosines not part of a CpG were used as an internal standard for unmethylated positions. The ratios of signal ratios for these cytosines had a mean of 1.59 and a standard deviation of 0.49 (n=20) and were distributed normally. In the H1299 sample, the values for all 16 cytosines in CpGs were at least four standard deviations from the mean of values for cytosines not in CpGs (FIG. 9A; Z scores listed in TABLE 2). A study in which the dye labels were reversed between the analyte and reference samples yielded equivalent results.

Specificity for Detection of Heterogeneous Methylation

The example of the present invention shows that the region of the p16 promoter is uniformly methylated at all CpG sites in the H1299 cell line. For non-uniformity of methylation that may have important biological consequences (e.g., because methylation of all CpG sites within a promoter region does not have equal effect on transcription), the ability for the assay to independently discriminate methylation states at different CpG sites is essential.

The present invention may detect methylation at an individual site and define the threshold for assignment of methylation state. This may be shown, for example, by creating an 190 base pair test duplex (using chemical synthesis and ligation). One strand of the duplex is identical in sequence to bisulfite-treated H69 genomic DNA, except the position of the 25th cytosine simulates methylation by being a cytosine rather than a thymidine. The test duplex was labeled by amplification with a labeled primer, and bisulfite-treated DNA from H69 lung tumor cells was amplified and labeled for use as a reference sequence. Co-hybridization of the analyte and reference samples to the array resulted in the ratios of analyte(C:T) to reference(C:T) listed in TABLE 2 for all 36 cytosines.

The site of simulated methylation had an analyte(C:T):reference(C:T) ratio of 2.38, nearly eight standard deviations (Z score=7.97) from the mean of that ratio for the cytosines not in CpG dinucleotides (1.13±0.16,n=20). This ratio for the other cytosines in CpGs ranged from 0.91 to 1.64. These differed from the mean for the internal standard cytosines by −1.8 to 3.6 standard deviations (FIG. 9B and TABLE 2). Thus, the authentic cytosine could be clearly distinguished from the other potential positions of methylation by its considerably larger variation from the internal standards. The range of ratios for the positions simulating unmethylated CpGs suggests a threshold Z score of greater than 3.6 (i.e., greater than 3.6 standard deviations from the mean of the internal standards) to indicate a genuine difference from an unmethylated cytosine. In FIG. 9, the threshold for calling methylation is set to 3.6, indicated by the horizontal line at that value. In each case the reference sample was derived from unmethylated DNA.

Detection of Methylated DNA in the Presence of Unmethylated DNA

The present invention is able to detect methylated cytosines within analytes that contain a significant amount of DNA that is not methylated, a feature that may be particularly useful with biological samples of genomic DNA that include individual CpG sites that are partially but not exhaustively methylated.

The 190 base region shown in FIG. 7 was amplified separately from bisulfite-treated samples of genomic DNA from H1299 and H69. The amount of amplified DNA from each sample was estimated by visualization on an agarose gel, and the amplified samples were mixed in a ratio of approximately 20:80 (H1299:H69). This mixture approximates a sample in which 20% of each CpG is methylated. The mixture was labeled by an additional amplification with a labeled primer. A reference sample (derived purely from H69) was also amplified and labeled, and the analyte mixture and reference were co-hybridized to the methylation probe array.

The results of this hybridization are summarized in TABLE 2. Of the 16 cytosines in CpG dinucleotides, 8 had Z scores greater than 3.6, identifying them as partially methylated (FIG. 9C). The remaining 8 could not be distinguished from bases converted entirely to deoxyuracils by treatment with bisulfite.

The comparison to a sample of reference methylation state is especially useful, because information about differences in methylation state is important. Many comparisons may be used, such as, for example, comparing the difference between the analyte sample and a sample known to be unmethylated, comparing DNA from diseased tissue to a matched sample from healthy tissue or DNA from tissue at different points along a disease progression. In FIG. 8C, co-hybridization with a reference sample containing a different label facilitates visualization of changes in methylation state; the presence of two colors in one set of four probes may then be observed.

Other aspects of variability of the present invention may be assessed using the known unmethylated positions as internal standards (generally performed after the context-dependence of variability is accounted for). For example, a calculated Z score offers a measure of the statistical significance of the difference between the analyte to reference ratio of a given interrogated cytosine and those known to be unmethylated. The use of an empirically determined threshold Z score to judge methylation state is analogous to the use of an empirically determined threshold signal ratio to identify nucleotides in standard array-based sequence analysis. As used herein, the calculated Z score correlates with methylation state, and a single cytosine corresponding to a uniquely methylated position is distinguished from the unmethylated cytosines.

The present invention may detect methylation at an individual cytosine by hybridization to probes synthesized in situ using internal controls such as cytosines outside of CpG dinucleotides and a co-hybridized reference sample. The assay is designed to interrogate independent sites for methylation. With use of the present invention, additional probes may be included to interrogate other possible strands of DNA that reflect methylation status of a region. For example, after bisulfite treatment, the two strands of genomic DNA are no longer mutually complementary. Amplification of each produces two complementary strands of different sequence. Therefore, information about the methylation state of the initial sequence is contained in four different sequences of DNA, each of which can be analyzed independently on the same array.

With the present invention, as few as two array features can be used to effectively probe each cytosine in a region of interest. For example, using light directed methods of high feature density array synthesis, hundreds of thousands of features can be created on a single array to probe, in parallel, hundreds of thousands of potential methylation sites in widely dispersed regions of the genome. This method of array synthesis that allows for high feature densities and facile changes in probe content is particularly valuable for the de novo discovery of sites of aberrant methylation states.

Although the invention has been described in connection with specific embodiments, it should be understood that the invention as claimed should not be unduly limited to such specific embodiments. Modifications and variations of the described compositions and methods of the invention will be apparent to those skilled in the art without departing from the scope and spirit of the invention. Indeed, various modifications of the described compositions and modes of carrying out the invention that are obvious to those skilled in molecular biology or related arts are intended to be within the scope of the following claims.

What is claimed is:

1. A method for the analysis of chemical modification of DNA comprising the steps of:
obtaining a sample of DNA to be analyzed;
treating the DNA with one or more chemical reagents that result in different base sequences depending upon the presence or absence of the modification of interest; and
determining a portion of the base sequence of the resulting DNA.

2. The method recited in claim 1, wherein the chemical modification of interest is methylation.

3. The method recited in claim 1, wherein the chemical modification of interest is methylation of cytosines.

4. The method recited in claim 1, wherein the chemical modification of interest is methylation of cytosines in CpG dinucleotides.

5. The method recited in claim 1, wherein the chemical modification of interest is methylation at the position of carbon 5 of cytosines.

6. The method recited in claim 1, wherein the chemical modification of interest is methylation of CpG dinucleotides within the promoter regions of one or more genes.

7. The method recited in claim 1, wherein the chemical modification of interest is methylation of CpG dinucleotides within the promoter regions of one or more tumor suppressor genes.

8. The method recited in claim 1, wherein the chemical modification of interest is methylation of CpG dinucleotides within the promoter regions of the tumor suppressor gene p16.

9. The method recited in claim 1, wherein the DNA is obtained from mammalian cells.

10. The method recited in claim 3, wherein the DNA is treated with reagents that convert unmethylated cytosines to deoxyuridines and leave methylated cytosines unchanged.

11. The method recited in claim 3, wherein the chemical reagents comprise bisulfite.

12. The method recited in claim 1, wherein part of the base sequence is determined by binding to an array comprising one or more probe molecules.

13. The method recited in claim 1, wherein the parts of the sequence that are determined comprise the base positions of potential modification.

14. The method recited in claim 12, wherein the probe molecules are DNA.

15. The method recited in claim 12, wherein the probe molecules comprise RNA, peptides, minor groove-binding polyamides, PNA, LNA, or 2′-O-methyl nucleic acid.

16. The method recited in claim 12, wherein the probe molecules comprise oligonucleotides.

17. The method recited in claim 16, wherein the probes comprise at least two oligonucleotides for every site in the sample to be analyzed.

18. The method recited in claim 12, wherein the probe molecules are immobilized on a solid substrate.

19. The method recited in claim 18, wherein the probe molecules are synthesized off of the array and subsequently deposited to the surface.

20. The method recited in claim 18, wherein the probes are synthesized directly on the surface of the array.

21. The method recited in claim 18, wherein the probes are synthesized by light-directed chemistry.

22. The method recited in claim 21, wherein the probes are synthesized using a digital micromirror array.

23. The method recited in claim 13, wherein the number of positions probed with a single array is greater than ten.

24. The method recited in claim 13, wherein the number of positions probed with a single array is greater than 100.

25. The method recited in claim 13, wherein the number of positions probed with a single array is greater than 1000.

26. The method recited in claim 13, wherein the number of positions probed with a single array is greater than 10000.

27. The method recited in claim 13, wherein the number of positions probed with a single array is greater than 100000.

28. The method recited in claim 1, wherein the part of the DNA for which modification is to be analyzed is determined by an automated search of a sequence database.

29. The method recited in claim 12, wherein the probe molecules are designed or selected by automated computational methods.

30. The method recited in claim 12, wherein binding is detected by fluorescence.

31. The method recited in claim 30, wherein the DNA to be applied to the array is labeled with a fluorescent dye.

32. The method recited in claim 31, wherein the fluorescent dye comprises a Cy family dye.

33. The method recited in claim 31, wherein a reference sample is labeled with a first dye and one or more samples to be analyzed are labeled with one or more second dyes.

34. The method recited in claim 33, wherein the reference sample is one for which the presence or absence of the modification of interest is known at each position of interest.

35. The method recited in claim 33, wherein the reference sample is from cells of a reference tissue.

36. The method recited in claim 33, wherein the reference sample has not been treated with chemical reagents that result in different base sequences depending upon the presence or absence of the modification of interest.

37. An array of one or more probes synthesized on a solid support wherein the probes are controlled for methylation state and detect one or more sites of methylation in a sample.

38. The array recited in claim 37, wherein the probes are complementary to the sites of methylation to be detected in the sample.

39. The array recited in claim 37, wherein the methylation site of interest consists of guanine.

40. The array recited in claim 37, wherein the methylation site of interest consists of adenosine.

41. The array recited in claim 37 further comprising one or more complementary nucleic acid sequences bound to one or more of the probes.

42. The array recited in claim 41, wherein the complementary nucleic acid sequence further comprises a fluorescent marker.

43. The array recited in claim 37, wherein the sample is DNA.

44. The array recited in claim 37, wherein the probe is selected from the group consisting of DNA, RNA, peptides, oligonucleotides, minor-groove binding polyamides, peptide nucleic acids, locked nucleic acids, 2′-O-methyl nucleic acids, and variations and combinations thereof.

45. The array recited in claim 37, wherein the probes are nucleic acid sequences of about 15 to about 30 bases in length.

46. A method for generating DNA probe sequences comprising the steps:
inputing a nucleic acid sequence in the 3-prime to 5-prime direction;
converting the sequence to account for chemical modification;
generating the complimentary sequence to the converted sequence in the 3-prime to 5-prime direction;
generating a first parent probe by choosing a first starting position on the complementary sequence and an first ending position on the complementary sequence;
generating a second parent probe by moving the first starting and first ending position one base unit in the same direction.

47. The method recited in claim 46, wherein the inputing is accomplished with a computer.

48. The method recited in claim 46, wherein the chemical modification comprises treatment with sodium bisulfite.

49. The method recited in claim 46, wherein the first starting position and the first ending position are separated by about 15 nucleic acid bases.

50. The method recited in claim 46, wherein the first starting position and the first ending position are separated by from about 15 nucleic acid bases to about 30 nucleic acid bases.

51. The method recited in claim 46 further comprising the step of filtering the parent probes to remove probes that are unsuitable for re-sequencing analysis.

52. The method recited in claim 51, wherein the filtering is based on low sequence complexity.

53. The method recited in claim 46 further comprising the step of using the first and second parent probes to generate additional probes by changing the nucleic acid nearest the midpoint to create a probe not already generated.

54. The method recited in claim 46 further comprising the step of outputting the parent probes generated to a computer file.

55. A method for generating DNA probe sequences comprising the steps:
inputing a nucleic acid sequence in the 3-prime to 5-prime direction;
converting the sequence to account for chemical modification;
generating the complimentary sequence to the converted sequence in the 3-prime to 5-prime direction;
locating one or more CpG dinucleotide regions within the complementary sequence;
generating one or more first probes by identifying sequences that have at least one nucleic acid on each end of the CpG dinucleotide regions.

56. The method recited in claim 55, wherein the inputing is accomplished with a computer.

57. The method recited in claim 55, wherein the chemical modification comprises treatment with sodium bisulfite.

58. The method recited in claim 55, wherein length of the probe is about 15 nucleic acid bases.

59. The method recited in claim 55, wherein the length of the probe is from about 15 nucleic acid bases to about 30 nucleic acid bases.

60. The method recited in claim 55 further comprising the step of filtering the parent probes to remove probes that are unsuitable for re-sequencing analysis.

61. The method recited in claim 55 further comprising the step of using the first probes to generate additional probes by changing the nucleic acid nearest the midpoint to create a probe not already generated.

60. The method recited in claim 55 further comprising the step of outputting the parent probes generated to a computer file.

61. An array with the DNA probe sequences of claim 55.

62. A method of preparing a probe for the analysis of chemical modifications of DNA comprising the steps of:
inputing a sample sequence as a sequence file into a computer;
converting the sequence file; and
generating a complementary sequence of the converted sequence file.

63. The method recited in claim 62, wherein the sample sequence is selected from the group consisting of DNA, RNA, peptides, oligonucleotides, minor-groove binding polyamides, peptide nucleic acids, locked nucleic acids, 2′-O-methyl nucleic acids, and variations and combinations thereof.

64. The method recited in claim 62, wherein the inputing of the sample sequence is in the five prime to three prime direction.

65. The method recited in claim 62, wherein the sequence file is converted to account for a chemical modification of the sample sequence.

66. The method recited in claim 62, wherein the complimentary sequence is generated in the five prime to three prime direction.

67. The method recited in claim 62 further comprises creating a parent probe list from the complementary sequence by standard re-sequencing and querying every position of the complemetary sequence.

68. The method recited in claim 67, wherein the parent probe list is filtered to remove unsuitable probes and is used to create a daughter list of probes containing one or more single polymorphisms at every position of each of the parent probes.

69. The method recited in claim 68, wherein a probe set is created that consists of all possible partners for each position and polymorphism.
